(19) United States
(12) Patent Application Publication
(10) Pub. No.: US 2011/0104788 A1
(43) Pub. Date: May 5, 2011

(54) Modulation of Adenoviral Tropism

(76) Inventors: Andrew Baker, Glasgow Lanarkshire (GB); Simon Waddington, London (GB); John McVey, London (GB)

(21) Appl. No.: 12/866,557

(22) PCT Filed: Feb. 9, 2009
(86) PCT No.: PCT/GB09/00363

§ 371 Date: Jan. 18, 2011

Related U.S. Application Data
(60) Provisional application No. 61/026,861, filed on Feb. 7, 2008.

(51) Int. Cl. C12N 7/00; C07K 14/075; C07H 21/00; C12N 15/63; C12N 7/01; C12N 5/10

(52) U.S. Cl. 435/235.1; 530/350; 536/23.72; 435/320.1; 435/325

(57) ABSTRACT

The invention provides materials and methods for modulating adenoviral tropism for hepatocytes and other cell types such as splenocytes. It relates to the findings that hypervariable regions (HVRs) of the viral hexon protein interact with the Gla domain of the blood clotting factor FX as part of the infective process in vivo. The invention provides means to disrupt the interaction between hexon and FX, thus reducing infection of hepatocytes and splenocytes, as well as use of targeting agents comprising the Gla domain or a fragment thereof to direct adenoviral vectors to desired target cell or tissue types.

### FIELD OF THE INVENTION

The invention relates to adenoviruses, and in particular to methods of reducing the tropism of adenoviruses for hepatic cells.

### BACKGROUND TO THE INVENTION

Adenoviruses are common pathogens used experimentally and in completed and ongoing clinical trials for gene delivery in oncology, cardioangiology, regenerative medicine and as vaccine vectors (Schenk-Braat et al., 2007; Kawabata et al, 2006). Much work has demonstrated the potential benefits and limitations of adenovirus-mediated gene therapy pre-clinically and clinically, the latter being brought to the forefront by the death of Jesse Gelsinger in 1999 in response to very high-dose adenovirus type 5 (Ad5) delivered directly into the hepatic artery (Raper et al., 2003). This dramatically highlighted the need to fully understand the virological and biological aspects that define adenovirus infectivity of liver and toxicity in vivo before vectors could be fully optimised for clinical use.

Adenoviruses based on human serotype 5 have been studied extensively, yet fundamental issues relating to infection mechanisms remain to be elucidated, particularly in vivo. Adenoviruses are principally made up of three major capsid proteins—hexon, penton and fiber. The crystal structures of Ad2 and Ad5 hexon have been solved (Athappilly et al., 1995; Roberts et al., 1986; Rux and Burnett, 2000) and reveal a complex structure. The hexon, the major site of antigenicity in adenoviruses (Pichla-Gollon, 2007; Roberts et al., 2006; Sumida et al., 2005), is the most abundant capsid protein and is positioned at the virion surface, arranged into 240 homotrimeric structures interlinked from neighbouring monomers, thus providing structural support. The fiber which engages the penton base at the capsid surface projects away from the virion (San Martin and Burnett, 2003), is highly variable in length, and is thought to be the major site determining cell infectivity [reviewed in (Nicklin et al., 2005)]. For subgroup C adenoviruses, including Ad5, the globular fiber knob positioned at the end of the trimeric fiber shaft binds the coxsackie and adenovirus receptor, expressed in an anatomically similar manner in mice and humans (Bergelson et al., 1997; Tomko et al., 1997), whereas subgroup B adenoviruses utilise CD46 which is ubiquitously expressed in humans but limited to the testes in mice (Gaggar et al., 2003; Segerman et al., 2003), Ad5 internalisation is mediated by integrin engagement with the penton base (Wickham et al., 1993). Extensive research efforts have primarily focused on the fiber protein for analysis of cell interactions in diverse gene therapy applications.

Following intravascular delivery, an important route of administration for many clinical applications, liver is the predominant site of Ad5 sequestration with substantial hepatocyte transduction (Huard et al., 1995). Ad5 vectors engineered through mutagenesis to lack CAR binding show identical liver transduction following intravascular injection in rodents and non-human primates (Alemany and Curiel, 2001; Smith et al., 2003) indicating alternate route(s) to gene transfer in vivo. A number of plasma proteins, including coagulation factor IX (FIX) and complement binding protein-4 (C4BP) were shown, using an ex vivo perfusion model, to bind the Ad fiber knob and “bridge” the virus to alternate receptors in liver, thus potentially bypassing CAR (Shayakhmetov et al., 2005). We showed using surface plasmon resonance (SPR) that Ad5 bound directly to the vitamin K-dependent coagulation factor X (FX) (Parker et al., 2006). In mice pretreated with warfarin (to ablate functional levels of vitamin K-dependent coagulation factors) liver infection was reduced by several orders of magnitude for both CAR-binding and CAR-binding mutant Ad5 vectors (Parker et al., 2006; Waddington et al., 2007) suggesting an important function for host factors in liver targeting. Injection of physiological levels of FX into warfarin-treated mice prior to intravenous Ad5 injection fully restored liver gene transfer (Parker et al., 2006; Waddington et al., 2007). FX has also been implicated in transduction of spleen (Waddington et al., 2007), which may also contribute to toxicity of adenoviral gene delivery vectors.

### SUMMARY OF THE INVENTION

The invention derives in part from the identification of the hexon, and in particular the hypervariable regions (HVRs) of the hexon, as the part of the virus primarily responsible for the interaction with Factor X (FX) which mediates adenoviral infection of hepatocytes in vivo, as well as other cell types whose transduction is mediated by FX, such as splenocytes. The inventors have also identified the Gla domain of FX as being involved in this interaction. These findings enable the provision, inter alia, of methods for modulating adenoviral transduction of hepatocytes and splenocytes, for example by stabilising, enhancing, inhibiting or disrupting the interaction between FX and adenovirus, methods of screening for novel agents capable of modulating this interaction, as well as novel adenoviruses having altered tropism for hepatocytes and splenocytes and methods for generation and identification of such novel viruses.

The invention provides a method of inhibiting FX-mediated transduction of a cell by an adenovirus, comprising contacting said cell with said adenovirus in the presence of an agent capable of inhibiting interaction between FX and adenoviral hexon protein.

Thus the invention provides a method of inhibiting transduction of a hepatocyte or splenocyte by an adenovirus, comprising contacting said hepatocyte or splenocyte with said adenovirus in the presence of an agent capable of inhibiting interaction between FX and adenoviral hexon protein.

It will be appreciated that the methods of the invention find particular utility in vivo, especially for reducing infection or transduction of a hepatocyte or splenocyte in a subject by an adenovirus which is to be deliberately administered to the subject, e.g. for therapeutic purposes. Consequently, the method may comprise administering said agent and said adenovirus to the subject.

It may be desirable to pretreat the subject with said agent prior to administration of the adenovirus, particularly where the agent binds to FX. The optimum pretreatment time may be determined depending on factors such as the identity and species of the subject, the identity of the agent, etc. Thus pretreatment may occur, for example, within one hour of administration, e.g. 30 minutes or less before administration. Alternatively it may be at least 1, 2, 3, 4, 5, 6 hours or more before said administration.

Alternatively it may be desirable to premix the agent with the adenovirus, particularly where the agent binds to the adenovirus. Mixing may be performed within one hour of administration, e.g. 30 minutes or less before administration. Alternatively it may be at least 1, 2, 3, 4, 5, 6 hours or more before said administration.

The invention also extends to methods performed in vitro or ex vivo. Such methods will be performed in an environment, typically culture medium, containing FX. FX may be added to the medium individually, or may be part of a bulk supplement to the medium such as serum (e.g. foetal calf serum, FCS).

For methods performed in vitro, it may be desirable for the adenovirus to contain a mutation inhibiting binding of the adenovirus to the coxsackie and adenovirus receptor (CAR). For example, this may facilitate study of the interaction between FX and Gla, by reducing the incidence of cellular infection by other mechanisms.

Alternatively it may be preferable to utilise a cell type having no or low CAR expression, such as SKOV3 cells.

In relation to methods performed either in vitro or in vivo, the inhibitory agent may bind to the Gla domain of FX.

For example, the agent may be FX-binding protein (X-bp) from Deinagkistrodon acutus, an analogue thereof, or a functional fragment of either which is capable of binding to FX and inhibiting interaction with hexon protein.

Another example of an inhibitory agent which binds to FX is an antibody specific for FX, and in particular an antibody specific for the Gla domain of FX. However it will be appreciated that it may not be necessary for the antibody to bind directly to the Gla domain in order for it to inhibit interaction between Gla and hexon. For example it may bind to another epitope of the FX molecule which is sufficiently close to the Gla domain in the folded molecule that the antibody sterically interferes with binding between Gla and hexon. Alternatively it may interfere with the interaction indirectly, e.g. by affecting the conformation or three-dimensional structure of the Gla molecule.

A further example is a soluble molecule comprising a fragment of adenoviral hexon protein capable of binding FX Gla. The molecule may be or may comprise a full length hexon protein.

Alternatively the inhibitory agent may bind to adenoviral hexon protein.

For example it may be an antibody specific for hexon protein, and in particular an antibody specific for the HVR of the hexon protein. As already explained above in relation to Gla, it will be appreciated that it may not be necessary for the antibody to bind directly to the HVR in order for it to inhibit interaction between Gla and hexon. For example it may bind to another epitope of the hexon molecule which is sufficiently close to the HVR domain in the folded molecule that the antibody sterically interferes with binding between Gla and hexon. Alternatively it may interfere with the interaction indirectly, e.g. by affecting the conformation or three-dimensional structure of the hexon molecule.

Another example of an inhibitory agent capable of binding to adenoviral hexon protein is a FX analogue which comprises a fragment of the Gla domain sufficient to bind to hexon protein but comprises a modification of the SP domain such that it is not capable of mediating interaction between adenovirus and hepatocyte or splenocyte. For example, it may comprise a mutation in the heparan sulphate proteoglycan-binding exocyte of the SP domain which reduces or ablates binding to hepatocytes or splenocytes. Alternatively the FX analogue may lack the SP domain completely. The FX analogue may comprise the full FX Gla domain but lack one or more domains including the SP domain, and optionally the EGF2 and/or EGF1 domains. For example, the FX analogue may comprise an FX component consisting of the Gla domain alone, Gla-EGF1 or Gla-EGF1-EGF2. The FX analogue may be a fusion protein comprising a FX component (e.g. Gla domain alone, Gla-EGF1 or Gla-EGF1-EGF2) and a heterologous fusion partner (i.e. a protein or protein domain not derived from FX).

The adenovirus or hexon protein may be of any suitable serotype. In some embodiments, it may be an Ad2 or Ad5 hexon protein.

The methods of the invention are particularly applicable to adenoviruses which are, or are intended for use as, gene transfer vectors.

The invention also provides the use of an agent capable of inhibiting the interaction between FX and adenoviral hexon protein in the manufacture of a medicament for inhibiting infection of a hepatocyte or splenocyte by an adenovirus.

The invention also provides an agent capable of inhibiting the interaction between FX and adenoviral hexon protein, for use in inhibiting infection of a hepatocyte or splenocyte by an adenovirus.

The invention also provides an agent capable of inhibiting the interaction between FX and adenoviral hexon protein, for use in a method of medical treatment.

Preferred features of these aspects of the invention are as set out above.

Inhibitory agents as described in this specification may also be formulated into pharmaceutical compositions and kits. Thus the invention provides a pharmaceutical preparation comprising an agent capable of inhibiting the interaction between FX and adenoviral hexon protein and an adenovirus, each in combination with a pharmaceutically acceptable carrier.

The inhibitory agent and the adenovirus may be formulated for administration separately or together, and in the same or different compositions.

Also provided is a kit comprising a first composition comprising an agent capable of inhibiting the interaction between FX and adenoviral hexon protein and an adenovirus and a second composition comprising an adenovirus. Preferably, each of the compositions is a pharmaceutical composition comprising the respective active component (inhibitory agent or adenovirus) and a pharmaceutically acceptable carrier. The compositions may be provided in separate containers, optionally with instructions for administration.

Knowledge of the interaction between hexon protein and the Gla domain of FX also enables methods of targeting adenoviruses to selected tissue or cell types. Thus the invention provides a method of delivering a gene to a target cell or tissue comprising contacting said cell or tissue with an adenoviral gene transfer vector and a targeting agent, wherein said targeting agent comprises a fragment of FX Gla domain capable of binding to a hexon protein of the adenoviral gene transfer vector associated with a binding agent capable of binding to said target cell or tissue.

The targeting agent may comprise a FX component comprising a complete FX Gla domain. The FX component may further comprise one or more further domains of FX, such as EGF1 and/or EGF2. Thus the FX component may comprise or consist of Gla-EGF1 or Gla-EGF1-EGF2. Typically the FX component does not comprise a FX SP domain. If a SP domain is present, it typically comprises a modification such that it is not capable of mediating interaction between adenovirus and hepatocyte or splenocyte, for example a mutation in the heparan sulphate proteoglycan-binding exocyte of the SP domain which reduces or ablates binding to hepatocytes or splenocytes.

The binding agent is heterologous to the FX component, i.e. it does not comprise components derived from FX. It is capable of binding to a binding partner present on or associated with the target cell or tissue. For example, it may comprise a ligand or receptor for a molecule expressed on the surface of the target cell or tissue, or a fragment thereof sufficient to bind. For example, it may comprise an antibody or aptamer specific for any molecule expressed on the surface of the cell or tissue, a ligand for a receptor expressed on the surface of the cell or tissue, a lectin capable of binding to a carbohydrate present on the surface of the cell or tissue, or any other suitable molecule.

The binding partner to which the binding agent binds may thus be a molecule expressed by the cell or tissue, and on the surface of the cell or tissue. Preferably it is specific to the target cell type, i.e. expressed exclusively or preferentially on the surface of the target cell. For example, where the target cell is a transformed cell (e.g. a cancer or tumour cell) it may be an antigen specific to that transformed cell. Such antigens are often referred to as tumour-specific antigens, although this term should not be taken to imply that the target cell must be a solid tumour. It may be any type of transformed cell.

The binding agent is preferably covalently linked to the FX component. In certain embodiments the targeting agent is a fusion protein comprising a single polypeptide chain comprising the binding agent and the FX component. A peptide linker may be present between the binding agent and the FX component.

It may be desirable to pretreat the subject with said targeting agent prior to administration of the adenovirus. The optimum pretreatment time may be determined depending on factors such as the identity and species of the subject, the identity of the targeting agent, etc. Thus pretreatment may occur, for example, within one hour of administration, e.g. 30 minutes or less before administration. Alternatively it may be at least 1, 2, 3, 4, 5, 6 hours or more before said administration.

Alternatively it may be desirable to premix the targeting agent with the adenovirus. Mixing may be performed within one hour of administration, e.g. 30 minutes or less before administration. Alternatively it may be at least 1, 2, 3, 4, 5, 6 hours or more before said administration.

The target cell is typically not a hepatocyte or splenocyte and the target tissue typically not liver or spleen. However the methods described may be applicable to increase the targeting of a gene transfer vector to hepatocytes or splenocytes, by use of a suitable binding agent.

The invention also provides the use of a targeting agent as described above in the manufacture of a medicament for gene transfer to a target cell or tissue by an adenovirus.

The invention also provides a targeting agent as described above for use in gene transfer to a target cell or tissue by an adenovirus.

The invention also provides a targeting agent as described above for use in a method of medical treatment.

Targeting agents as described above may also be formulated into pharmaceutical compositions and kits. Thus the invention provides a pharmaceutical preparation comprising a targeting agent as described above and an adenovirus, each in combination with a pharmaceutically acceptable carrier.

The targeting agent and the adenovirus may be formulated for administration separately or together, and in the same or different compositions.

Also provided is a kit comprising a first composition comprising a targeting agent as described above and a second composition comprising an adenovirus. Preferably, each of the compositions is a pharmaceutical composition comprising the respective active component (targeting agent or adenovirus) and a pharmaceutically acceptable carrier. The compositions may be provided in separate containers, optionally with instructions for administration.

The adenoviral hexon protein (in particular the HVRs) and the Gla domain have been identified as significant factors mediating the interaction between adenovirus and FX. This therefore enables screening for agents capable of modulating that interaction. For example, agents capable of inhibiting or disrupting that interaction would find use in methods of reducing adenoviral transduction of hepatocytes and other cell types whose transduction is mediated by FX such as splenocytes, as described above. In contrast, agents capable of promoting (e.g. stabilising or enhancing) that interaction would find use in targeting adenoviral vectors to hepatocytes for gene transfer to the liver, and cell types such as splenocytes for gene transfer to other tissues such as spleen, e.g. for treatment of liver dysfunction or infection.

The invention therefore provides a method of screening for an agent capable of modulating FX-mediated transduction, e.g. of a hepatocyte or splenocyte, by an adenovirus, comprising contacting a test agent with a first substance comprising a fragment of an adenoviral hexon protein capable of binding FX, and a second substance comprising a fragment of FX Gla domain capable of binding adenoviral hexon protein, and determining binding between said hexon fragment and said FX Gla fragment.

The first substance may comprise any fragment of a hexon protein capable of binding to FX Gla. For example, it may comprise a single isolated HVR, or two or more (e.g. 2, 3, 4, 5, 6 or 7) HVRs optionally with intervening scaffold sequence, or a complete hexon protein.

It may be a wild type hexon protein or fragment thereof, from any suitable serotype, such as Ad2 or Ad5. Alternatively it may contain one or more mutations or modifications in the scaffold or HVRs relative to a wild type hexon protein. For example it may contain one or more point mutations in the HVRs which modulate binding to FX relative to a corresponding wild type HVR.

For example it may contain one or more mutations (e.g. substitution, deletion or addition) at residues corresponding to Thr268, Thr269 and/or Glu270 of Ad5 hexon protein [shown as Thr269, Thr270 and Glu271 in the sequence provided below having accession nos. AAW65514.1; GI:58177707]. These are located in HVR5. It may contain one or more mutations at residues corresponding to Ile420, Asn421, Thr422, Glu423, Thr424, Leu425, Asp447 or Lys448 of Ad5 hexon protein [shown as Ile421, Asn422, Thr423, Glu424, Thr425, Leu426, Asp448 and Lys449 in the sequence provided below having accession nos. AAW65514.1; GI:58177707].

These are located in HVR7. Additionally or alternatively it may contain one or more mutations at residues corresponding to Glu450, Arg452 and Val453 of Ad5 hexon protein [shown as Glu451, Arg453 and Val454 in the sequence provided below having accession nos. AAW65514.1; GI:58177707]. These are located in scaffold sequence flanking HVR7. Additionally or alternatively it may comprise one or more mutations at residues corresponding to Glu212, Thr213, Glu214, Ile215, Asn216, Ser267, Met314, Asn421, Thr426, Ser446 or Asn449 of Ad5 hexon protein [shown as Glu213, Thr214, Glu215, Ile216, Asn217, Ser268, Met315, Asn422, Thr427, Ser447 and Asn450 in the sequence provided below having accession nos. AAW65514.1; GI:58177707]. Any mutation which modulates, especially reduces, binding to FX may be used. For example, a parent or wild type residue may be replaced by the residue occurring at the corresponding position in an adenovirus from a serotype with a lower affinity for FX or a lower capacity to transduce hepatocytes or splenocytes. For example, a residue in Ad5 may be exchanged for a residue from one of Ad17, Ad20, Ad25, Ad26, Ad28, Ad29, Ad44 or Ad48.

It may contain mutations at residues corresponding to one or both of Thr269 and Glu270 of Ad5 [shown as Thr270 and Glu271 in the sequence having accession nos. AAW65514.1; GI:56177707]. It may be desirable to introduce Pro or Asp at a position corresponding to Thr269. It may be desirable to introduce a Gly or Ser residue at a position corresponding to Glu270. Thus, for example, it may contain substitutions corresponding to Thr269Pro and/or Glu270Gly in Ad5.

It may contain mutations at residues corresponding to one, two three or all four of Ile420, Thr422. Glu423 and Leu425 [shown as Ile421, Thr423. Glu424 and Leu426 in the sequence having accession nos. AAW65514.1; GI:58177707]. It may be desirable to introduce Gly at a position corresponding to Ile420. Additionally or alternatively, it may be desirable to introduce Asn at a position corresponding to Thr422. Additionally or alternatively, it may be desirable to introduce Ser or Ala at a position corresponding to Glu423. Additionally or alternatively, it may be desirable to introduce Tyr at a position corresponding to Leu425. For example it may contain substitutions corresponding to one, two, three or all four of the substitutions Ile420Gly, Thr422Asn, Glu423Ser and Leu425Tyr. It may also be desirable to introduce Val or Ala at a position corresponding to Thr424 of Ad5.

A particularly desirable position to introduce a mutation may be a position corresponding to Glu450 of Ad5 [shown as Glu451 in the sequence provided below having accession nos. AAW65514.1; GI:58177707]. This is located in the final scaffold region of the protein, 6 residues downstream of HVR7. It may be desirable to introduce a neutral or positively charged residue at that position. In particular it may be desirable to replace a Glu residue occurring naturally at such a position with another residue, preferably a neutral or positively charged residue, for example Gln, Ile, Leu, Val, Arg or Lys, e.g. Gln or Arg.

The second substance may contain a scaffold from a first adenoviral serotype and one or more HVRs from a different adenoviral serotype. Where two or more HVRs are present, each HVR may be from a different serotype. Typically the first substance does not comprise at least one of adenoviral fiber protein and penton protein. In some embodiments it does not comprise either adenoviral penton protein or fiber protein.

Thus it may include a scaffold from Ad2 or Ad5, and one or more HVRs from at least one of Ad17, Ad20, Ad25, Ad26, Ad28, Ad29, Ad44 or Ad48. In some embodiments, at least HVR5 and/or HVR7 are derived from Ad17, Ad20, Ad25, Ad26, Ad28, Ad29, Ad44 or Ad48, for example from Ad26 or Ad48.

They may also be derived from another group D serotype such as Ad9, Ad10, Ad15, Ad19, Ad22, Ad24, Ad27, Ad30, Ad32, Ad33, Ad36, Ad38, Ad39, Ad42, Ad43, Ad45, Ad47 or Ad51.

The second substance may comprise or consist of any suitable fragment of FX comprising the Gla domain or a fragment thereof capable of binding hexon protein. Thus, the second substance may comprise or consist of FX Gla-EGF1, Gla-EGF1-EGF2, or Gla-EGF1-EGF2-SP. In some embodiments, the second substance is not full-length FX.

The second substance may be a fusion protein containing a FX component and a heterologous fusion partner (i.e. a protein or protein domain not derived from FX). The FX component comprises a FX Gla domain, and may comprise one or more of the EGF1, EGF2 and SP domains. For example it may comprise Gla-EGF1 or Gla-EGF1-EGF2.

To facilitate the analysis, either the first or second substance may be attached (e.g. covalently linked) to a solid support. Thus the method may comprise providing the first or second substance attached to a solid support, contacting the solid support with a sample (typically a liquid sample) containing the second or first substance respectively, and determining the amount of second or first substance associated with the support.

One or more washing steps may be included between the contacting and determining steps to reduce or minimise the amount of second or first substance non-specifically associated with the support. Binding between the first and second substances may be determined directly, or indirectly. For example, the second substance may be labelled, e.g. with a radioactive or spectrophotometrically detectable probe (e.g. a fluorescent probe). Alternatively the method may involve contacting the solid support with a further binding agent capable of detecting a complex between the first and second agents, such as an antibody specific for the substance not attached to the support. The binding agent may itself be labelled.

The amount of binding detected will thus depend on the ability of the test agent to disrupt or prevent binding.

The skilled person will be aware of numerous techniques and assay formats which would be suitable, including ELISA, surface plasmon resonance, etc.

The method may comprise determining binding between the hexon fragment and the FX Gla fragment in the presence and absence of the test agent, and selecting the test agent if binding is different (e.g. lower or higher) in the presence of the agent than in the absence of the agent.

The test agent may be any suitable molecule, including protein, carbohydrate, small molecule (e.g. having a molecular mass of less than 500 Da), etc. For example it may be an antibody, an analogue of X-bp, an isolated Gla domain or fragment or derivative thereof, an isolated hexon protein or fragment or derivative thereof, etc.

The method may comprise testing a plurality of test agents, which may or may not be structurally related to one another. A high throughput format is preferably used. The plurality of test agents may be a library of small molecules, or a plurality of protein molecules, for example a plurality of analogues or variants of a known protein such as X-bp.

The method may also include a positive control using a known agent capable of modulating interaction between HVR and Gla. For example, X-bp may be used as a suitable control in an assay intended to identify an agent capable of inhibiting or disrupting the interaction. Thus the efficacy of any given test agent may be compared directly with that of a known control agent.

Having identified or selected a suitable test agent, it may be desirable to confirm its ability to modulate adenoviral transduction of hepatocytes or splenocytes. Thus the method may additionally comprise the step of contacting the test agent with a hepatocyte or splenocyte and an adenovirus and determining transduction of the hepatocyte or splenocyte by the adenovirus. Typically the adenovirus will contain the same hexon fragment as the first substance described above.

The method may comprise comparing transduction of the hepatocyte or splenocyte by the adenovirus in the presence and absence of the test agent. It may also comprise a positive control using a known agent capable of modulating interaction between HVR and Gla, such as X-bp.

The efficacy of the test agent in modulating adenoviral infection of liver or spleen may be tested in vivo or in vitro. When performed in vitro the adenovirus may comprise a mutation which reduces CAR binding. Alternatively it may be preferable to utilise a cell type having no or low CAR expression, such as SKOV3 cells.

Tests in vivo may be performed on any suitable model, such as a rodent or non-human primate.

The adenovirus used in the confirmatory tests in vitro or in vivo may be a gene transfer vector carrying a gene (e.g. a marker gene) whose expression in transduced hepatocytes or splenocytes is readily detectable. Thus the method may comprise the step of detecting an expression product of said gene, directly or indirectly, e.g. in the hepatocyte or splenocyte, on the surface of the hepatocyte or splenocyte, or secreted from the hepatocyte or splenocyte. Direct detection may comprise detection of protein or mRNA, e.g. using a binding agent capable of binding to the expression product. Indirect detection may comprise detecting the presence of a product produced only in the presence of the expression product. Suitable marker genes include those encoding enzymes, fluorescent proteins, cell-surface receptors, etc.

The findings described in this specification also enable the generation of adenoviral hexon proteins having altered affinity for FX, which may then be used to create adenoviruses, e.g. gene transfer vectors, having altered ability to transduce cells whose transduction is mediated in whole or in part by FX, e.g. hepatocytes or splenocytes. Those with reduced affinity for FX may be used to provide vectors displaying improved gene targeting to cell and tissue types other than liver and perhaps spleen, as well as having improved safety profiles. Those with increased affinity for FX may be useful in vectors intended for gene transfer to hepatocytes or splenocytes, e.g. for treatment of liver dysfunction or infection.

Thus the invention provides a method of screening for an adenoviral hexon protein having altered affinity for FX, comprising:

providing a substance comprising a fragment of an adenoviral hexon protein capable of binding FX;
contacting said substance with a fragment of FX Gla domain capable of binding to adenoviral hexon; and
determining binding between said hexon fragment and the FX Gla fragment.

The substance may comprise or consist of any fragment of a hexon protein capable of binding to FX Gla. For example, it may comprise a single isolated HVR, two or more (e.g. 2, 3, 4, 5, 6 or 7) HVRs optionally with intervening scaffold sequence, or a complete adenoviral hexon protein. Typically it does not comprise at least one of adenoviral fiber protein and penton protein. In some embodiments it does not comprise either adenoviral penton protein or fiber protein.

Typically the method will involve comparing binding between two or more such substances each containing hexon fragments having different sequences to one another. Thus the method may comprise the steps of:

providing at least first and second substances each comprising a respective first and second adenoviral hexon fragment;
contacting each said substance with a fragment of FX Gla domain capable of binding hexon protein; and
determining binding between each said hexon fragment and the FX Gla fragment.

Thus the first substance may comprise a reference hexon sequence, e.g. one or more isolated HVRs or a full length hexon protein, having known Gla-binding properties (or FX-binding properties). The second substance (and any subsequent substances) may comprise a test sequence, e.g. isolated HVR or hexon protein, whose Gla or FX-binding properties are to be compared with the reference.

It may be desirable for the first and second substances to be substantially identical apart from their HVR sequences, in order to increase the chance that any difference in binding properties is due to the difference between HVRs.

Thus the first hexon fragment may have a wild type sequence. The second hexon fragment may have a sequence not found in nature. For example, its amino acid sequence may comprise one or more mutations or modifications relative to the first sequence, e.g. in an HVR or one or more scaffold amino acids. For example it may have one or more mutations or modifications in one or more of HVRs 3, 5 and 7.

For example it may contain one or more mutations (e.g. substitution, deletion or addition) at residues corresponding to Thr268, Thr269 and/or Glu270 of Ad5 hexon protein [shown as Thr269, Thr270 and Glu271 in the sequence provided below having accession nos. AAW65514.1; GI:58177707]. These are located in HVR5. It may contain one or more mutations at residues corresponding to Ile420, Asn421, Thr422, Glu423, Thr424, Leu425, Asp447 or Lys448 of Ad5 hexon protein [shown as Ile421, Asn422, Thr423, Glu424, Thr425, Leu426, Asp448 or Lys449 in the sequence provided below having accession nos. AAW65514.1; GI:58177707]. These are located in HVR7. Additionally or alternatively it may contain one or more mutations at residues corresponding to Glu450, Arg452 and VAl453 of Ad5 hexon protein [shown as Glu451, Arg453 and VAl454 in the sequence provided below having accession nos. AAW65514.1; GI:58177707]. These are located in scaffold sequence flanking HVR7. Additionally or alternatively it may comprise one or more mutations at residues corresponding to Glu212, Thr213, Glu214, Ile215, Asn216, Ser267, Met314, Asn421, Thr426, Ser446 or Asn449 of Ad5 hexon protein [shown as Glu213, Thr214, Glu215, Ile216, Asn217, Ser268, Met315, Asn422, Thr427, Ser447 or Asn450 in the sequence provided below having accession nos. AAW65514.1; GI:58177707]. These residues are believed to be particularly important in the interaction between hexon and FX. Any mutation which modulates, especially reduces, binding to FX may be used. For example, a parent or wild type residue may be replaced by the residue occurring at the corresponding position in an adenovirus from a serotype with a lower affinity for FX or a lower capacity to transduce hepatocytes or splenocytes. For example, a residue in Ad5 may be exchanged for a residue from one of Ad17, Ad20, Ad25, Ad26, Ad28, Ad29, Ad44 or Ad48.

It may contain mutations at residues corresponding to one or both of Thr269 and Glu270 of Ad5 [shown as Thr270 and Glu271 in the sequence having accession nos. AAW65514.1; GI:58177707]. It may be desirable to introduce Pro or Asp at a position corresponding to Thr269. It may be desirable to introduce a Gly or Ser residue at a position corresponding to Glu270. Thus, for example, it may contain substitutions corresponding to Thr269Pro and/or Glu270Gly in Ad5.

It may contain mutations at residues corresponding to one, two three or all four of Ile420, Thr422. Glu423 and Leu425 [shown as Ile421, Thr423. Glu424 and Leu426 in the sequence having accession nos. AAW65514.1; GI:58177707]. It may be desirable to introduce Gly at a position corresponding to Ile420. Additionally or alternatively, it may be desirable to introduce Asn at a position corresponding to Thr422. Additionally or alternatively, it may be desirable to introduce Ser or Ala at a position corresponding to Glu423. Additionally or alternatively, it may be desirable to introduce Tyr at a position corresponding to Leu425. For example it may contain substitutions corresponding to one, two, three or all four of the substitutions Ile420Gly, Thr422Asn, Glu423Ser and Leu425Tyr. It may also be desirable to introduce Val or Ala at a position corresponding to Thr424 of Ad5.

A particularly desirable position to introduce a mutation may be a position corresponding to Glu450 of Ad5 [shown as Glu451 in the sequence provided below having accession nos. AAW65514.1; GI:58177707]. This is located in the final scaffold region of the protein, 6 residues downstream of HVR7. It may be desirable to introduce a neutral or positively charged residue at that position. In particular it may be desirable to replace a Glu residue occurring naturally at such a position with another residue, preferably a neutral or positively charged residue, for example Gln, Ile, Leu, Val, Arg or Lys, e.g. Gln or Arg.

Where the two substances comprise or consist of hexon proteins, the reference hexon sequence may comprise a wild type hexon protein. The test hexon sequence may comprise a chimeric hexon protein in which the scaffold and one or more HVRs are derived from different wild type hexon proteins. The scaffold may be from the reference hexon protein. For example, one or more of HVRs 3, 5 and 7 may be derived from a different serotype than the scaffold. The HVRs may be derived from a hexon protein from a serotype having lower hepatic tropism than the serotype from which the scaffold is derived.

The reference hexon sequence may be of serotype Ad2 or Ad5.

The test hexon sequence may be of serotype Ad17, Ad20, Ad25, Ad26, Ad28, Ad29, Ad44 or Ad48. If the test sequence is a chimeric sequence it may comprise a scaffold fragment from Ad2 or Ad5 and at least one HVR sequence from at least one of Ad17, Ad20, Ad25, Ad26, Ad28, Ad29, Ad44 or Ad48. The HVR sequence may also be derived from another group D serotype such as Ad9, Ad10, Ad15, Ad19, Ad22, Ad24, Ad27, Ad30, Ad32, Ad33, Ad36, Ad38, Ad39, Ad42, Ad43, Ad45, Ad47 or Ad51.

Thus it may include a scaffold region from Ad2 or Ad5, and one or more HVRs from at least one of Ad17, Ad20, Ad25, Ad26, Ad28, Ad29, Ad44 or Ad48. In some embodiments, HVR3, HVR5 and/or HVR7 are derived from Ad17, Ad20, Ad25, Ad26, Ad28, Ad29, Ad44 or Ad48, for example from Ad26 or Ad48. They may also be derived from another group D serotype such as Ad9, Ad10, Ad15, Ad19, Ad22, Ad24, Ad27, Ad30, Ad32, Ad33, Ad36, Ad38, Ad39, Ad42, Ad43, Ad45, Ad47 or Ad51.

Thus the second or test fragment may be a deliberately modified form of the first or reference fragment. This can be achieved by engineering a nucleic acid encoding the first or reference fragment, in order to generate a second or test fragment, e.g. comprising one or more different HVRs. Preferably only an HVR sequence (and/or one or more flanking amino acids) is modified. In such cases, the method may thus comprise providing a first nucleic acid sequence encoding said first substance, and modifying said first nucleic acid to generate a second nucleic acid sequence encoding said second substance.

It will be appreciated that a library of test fragments may be generated. Thus the method may further comprise mutating said first nucleic acid sequence to generate a plurality of test nucleic acid sequences (e.g. at least 5, at least 10, at least 50, at least 100, at least 1000, at least 10,000, at least 10⁶ or even more test sequences) each encoding a test fragment, and wherein the test fragments are different to one another.

Mutations or modifications may be made at specifically targeted positions or at random positions, e.g. within an HVR. In either case they may involve introducing random substituents or specific amino acids. For example, a plurality of nucleic acids encoding HVR sequences (optionally including one or more flanking amino acids), differing from one another at one or more positions, may be introduced into a population of nucleic acids encoding an identical scaffold sequence, thus providing a library of different HVRs for screening.

The screening may be carried out by assay methods as described above, or by high throughput methods such as phage display, etc.

Having identified or selected a suitable hexon fragment having desired Gla or FX-binding characteristics, it may be desirable to test transduction of a hepatocyte or splenocyte by an adenovirus comprising a hexon protein comprising that fragment. The method may therefore comprise selecting a substance containing a fragment having desired binding characteristics to Gla or FX, generating an adenovirus containing a hexon protein comprising the respective fragment, contacting said adenovirus and a hepatocyte or splenocyte, and determining transduction of the hepatocyte or splenocyte by the adenovirus.

The infectivity of the adenovirus may be tested in vivo or in vitro. When tested in vitro the adenovirus may comprise a mutation which reduces CAR binding. Alternatively it may be preferable to utilise a cell type having no or low CAR expression, such as SKOV3 cells.

Tests in vivo may be performed on any suitable model, such as a rodent or non-human primate.

The adenovirus used in the confirmatory tests in vitro or in vivo may be a gene transfer vector carrying a gene whose expression in transduced hepatocytes or splenocytes is readily detectable. The method may comprise the step of detecting an expression product of said gene, e.g. in the hepatocyte or splenocyte, on the surface of the hepatocyte or splenocyte, or secreted from the hepatocyte or splenocyte. Detection may be direct or indirect.

Whichever test system is used, it may be desirable to compare the ability of the adenovirus to transduce hepatocytes or splenocytes with that of an adenovirus comprising a hexon protein containing the first or reference hexon fragment.

In some circumstances it may be desirable not to test particular hexon proteins for their binding properties against isolated Gla or FX, but simply to test the ability of an adenovirus having a particular hexon sequence to transduce cells whose transduction is mediated by FX, e.g. hepatocytes or splenocytes. Typically, this will be compared to a reference adenovirus having a known reference hexon sequence and whose transduction properties are known.

The invention therefore provides a method of screening for an adenovirus having altered ability to transduce hepatocytes or splenocytes comprising

providing a first adenovirus comprising a first hexon protein;
providing a second adenovirus comprising a second hexon protein; and
comparing the ability of said first and said second adenoviruses to transduce hepatocytes or splenocytes.

The first and second hexon proteins have different sequences. Typically, the first and second hexon proteins differ in one or more HVR sequences and/or one or more amino acids in the scaffold sequence. It may be desirable for the first and second hexon proteins to be substantially identical apart from said one or more HVRs or scaffold residues. It may also be desirable that the first and second adenoviruses are identical in all other respects in order to increase the chance that any difference in binding or transduction properties can be confidently ascribed to the difference between the hexon proteins. For example, they may possess the same penton and fiber proteins.

Thus the first hexon protein may comprise a reference HVR, having known Gla-binding properties (or FX-binding properties). The second hexon protein (and any subsequent hexon proteins) may comprise a test HVR, whose Gla or FX-binding properties are to be compared with the reference.

Thus the reference HVR may be a wild type HVR. The test HVR may have a sequence not found in nature. For example, its amino acid sequence may comprise one or more mutations or modifications relative to the reference HVR, e.g. in one or more of HVRs 3, 5 and/or 7.

For example the test hexon protein may contain one or more mutations (e.g. substitution, deletion or addition) at residues corresponding to Thr268, Thr269 and/or Glu270 of Ad5 hexon protein [shown as Thr269, Thr270 and Glu271 in the sequence provided below having accession nos. AAW65514.1; GI:58177707]. These are located in HVR5. It may contain one or more mutations at residues corresponding to Ile420, Asn421, Thr422, Glu423, Thr424, Leu425, Asp447 or Lys448 of Ad5 hexon protein [shown as Ile421, Asn422, Thr423, Glu424, Thr425, Leu426, Asp448 and Lys449 in the sequence provided below having accession nos. AAW65514.1; GI:58177707]. These are located in HVR7. Additionally or alternatively it may contain one or more mutations at residues corresponding to Glu450, Arg452 and Val453 of Ad5 hexon protein [shown as Glu451, Arg453 and Val454 in the sequence provided below having accession nos. AAW65514.1; GI:58177707]. These are located in scaffold sequence flanking HVR7. Additionally or alternatively it may comprise one or more mutations at residues corresponding to Glu212, Thr213, Glu214, Ile215, Asn216, Ser267, Met314, Asn421, Thr426, Ser446 or Asn449 of Ad5 hexon protein [shown as Glu213, Thr214, Glu215, Ile216, Asn217, Ser268, Met315, Asn422, Thr427, Ser447 and Asn450 in the sequence provided below having accession nos. AAW65514.1; GI:58177707]. For example, a parent or wild type residue may be replaced by the residue occurring at the corresponding position in an adenovirus from a serotype with a lower affinity for FX or a lower capacity to transduce hepatocytes or splenocytes. For example, a residue in Ad5 may be exchanged for a residue from one of Ad17, Ad20, Ad25, Ad26, Ad28, Ad29, Ad44 or Ad48.

It may contain mutations at residues corresponding to one or both of Thr269 and Glu270 of Ad5 [shown as Thr270 and Glu271 in the sequence having accession nos. AAW65514.1; GI:58177707]. It may be desirable to introduce Pro or Asp at a position corresponding to Thr269. It may be desirable to introduce a Gly or Ser residue at a position corresponding to Glu270. Thus, for example, it may contain substitutions corresponding to Thr269Pro and/or Glu270Gly in Ad5.

It may contain mutations at residues corresponding to one, two three or all four of Ile420, Thr422. Glu423 and Leu425 [shown as Ile421, Thr423, Glu424 and Leu426 in the sequence having accession nos. AAW65514.1; GI:58177707]. It may be desirable to introduce Gly at a position corresponding to Ile420. Additionally or alternatively, it may be desirable to introduce Asn at a position corresponding to Thr422. Additionally or alternatively, it may be desirable to introduce Ser or Ala at a position corresponding to Glu423. Additionally or alternatively, it may be desirable to introduce Tyr at a position corresponding to Leu425. For example it may contain substitutions corresponding to one, two, three or all four of the substitutions Ile420Gly, Thr422Asn. Glu423Ser and Leu425Tyr. It may also be desirable to introduce Val or Ala at a position corresponding to Thr424 of Ad5.

A particularly desirable position to introduce a mutation may be a position corresponding to Glu450 of Ad5 [shown as Glu451 in the sequence provided below having accession nos. AAW65514.1; GI:58177707]. This is located in the final scaffold region of the protein, 6 residues downstream of HVR7. It may be desirable to introduce a neutral or positively charged residue at that position. In particular it may be desirable to replace a Glu residue occurring naturally at such a position with another residue, preferably a neutral or positively charged residue, for example Gln, Ile, Leu, Val, Arg or Lys, e.g. Gln or Arg.

The reference hexon protein may be a wild type hexon protein. The test hexon protein may be a chimeric hexon protein in which the scaffold and one or more HVRs are derived from different wild type hexon proteins. The scaffold may be from the reference hexon protein.

For example, one or more of HVRs 3, 5 and 7 may be derived from a different serotype than the scaffold. The HVRs may be derived from a hexon protein from a serotype having lower hepatic tropism than the serotype from which the scaffold is derived.

The reference HVR or hexon protein may be of serotype Ad2 or Ad5.

The test HVR or hexon protein may be of serotype Ad17, Ad20, Ad25, Ad26, Ad28, Ad29, Ad44 or Ad48. Where it is chimeric, the scaffold may be from Ad2 or Ad and one or more HVRs from Ad17, Ad20, Ad25, Ad26, Ad28, Ad29, Ad44 or Ad48. The HVRs may also be derived from another group D serotype such as Ad9, Ad10, Ad15, Ad19, Ad22, Ad24, Ad27, Ad30, Ad32, Ad33, Ad36, Ad38, Ad39, Ad42, Ad43, Ad45, Ad47 or Ad51.

Thus the test hexon protein may include a scaffold region from Ad2 or Ad5, and one or more HVRs from at least one of Ad17, Ad20, Ad25, Ad26, Ad28, Ad29, Ad44 or Ad48. In some embodiments, HVR5 and/or HVR7 are derived from Ad17, Ad20, Ad25, Ad26, Ad28, Ad29, Ad44 or Ad48, for example from Ad26 or Ad48. They may also be derived from another group D serotype such as Ad9, Ad10, Ad15, Ad19, Ad22, Ad24, Ad27, Ad30, Ad32, Ad33, Ad36, Ad38, Ad39, Ad42, Ad43, Ad45, Ad47 or Ad51.

Thus the second or test hexon protein may be a deliberately modified form of the first or reference hexon protein. This can be achieved by engineering a nucleic acid sequence encoding the first or reference hexon protein, in order to generate a second or test hexon protein having a different sequence. Preferably only one or more HVR regions and/or flanking amino acids are modified. In such cases, the method may thus comprise providing a first nucleic acid sequence encoding said first or reference hexon protein, and modifying said first nucleic acid to generate a second nucleic acid sequence encoding said second or test hexon protein.

It will be appreciated that a library of test proteins may be generated. Thus the method may further comprise mutating said first nucleic acid sequence to generate a plurality of test nucleic acid sequences (e.g. at least 5, at least 10, at least 50, at least 100, at least 1000, at least 10,000, at least 10⁶ or even more test sequences) each encoding a test hexon protein, wherein the test hexon proteins are different to one another.

Mutations or modifications may be made at specifically targeted positions or at random positions, e.g. within an HVR and/or at positions set out above. In either case they may involve introducing random substituents or specific amino acids. For example, a plurality of nucleic acids encoding HVR sequences (optionally including one or more flanking amino acids), differing from one another at one or more positions, may be introduced into a population of nucleic acids encoding an identical scaffold sequence, thus providing a library of hexon proteins containing different HVRs for screening.

The method may comprise contacting at least respective first and second hepatocytes, or at least respective first and second splenocytes, with at least said first and second adenoviruses in vitro in the presence of FX and determining transduction of said cells by said adenoviruses.

In such assays, the adenoviruses may comprise a mutation which reduces binding to CAR. Alternatively it may be preferable to utilise a cell type having low or no CAR expression, such as SKOV3 cells.

Alternatively the method may be performed in vivo and may therefore comprise administration of said first and second adenoviruses to respective first and second (preferably non-human) subjects and determining transduction of hepatocytes or splenocytes.

A suitable subject may be a rodent or non-human primate.

The method may comprise selecting an adenovirus having most significantly altered (e.g. enhanced or reduced) transduction.

The method may be used to identify a particular hexon fragment, e,g, HVR sequence or set of HVR sequences and/or scaffold sequences with particular binding to FX. It may then be desirable to introduce those sequences into a hexon protein of a different adenovirus, e.g. one of a different serotype, or simply to introduce the hexon protein containing the selected HVR sequence into a different adenovirus. The resulting adenovirus may then be tested for transduction ability towards hepatocytes or splenocytes in the presence of FX as described above, optionally being compared with a suitable reference adenovirus.

It will be apparent that the findings described here also make available methods of preparing novel adenoviruses having altered tropism for cell types whose transduction is mediated by FX, such as hepatocytes or splenocytes.

Thus the invention provides a method of modulating liver or spleen infectivity of an adenovirus comprising:

providing a parent adenoviral hexon protein;
modifying said parent hexon protein to produce a modified hexon protein having altered affinity for FX; and
producing an adenovirus comprising said modified hexon protein;
whereby said adenovirus has altered ability to transduce hepatocytes or splenocytes compared to an adenovirus comprising said parent hexon protein. Thus the adenovirus comprising the modified hexon protein has altered (i.e. increased or decreased) ability to transduce hepatocytes or splenocytes compared to a reference adenovirus is which identical in all respects apart from the modifications introduced into the hexon protein. In particular the two viruses will share the same penton and fiber proteins.

The invention also extends to the products of such methods. Thus the invention provides an adenovirus containing a modified hexon protein which comprises a mutation relative to a parent hexon protein, whereby said adenovirus has altered ability to transduce hepatocytes or splenocytes compared to an adenovirus comprising said parent hexon protein, with the proviso that the modified hexon protein is not Ad5 hexon protein comprising HVR from Ad48 hexon protein. In particular the modified hexon protein is not Ad5 hexon protein comprising HVR1 from Ad48 hexon protein and wild type Ad5 HVR2-7, and is not Ad5 hexon protein comprising HVRs 1-7 from Ad48 hexon protein, as described in Reynolds et al., 2006.

The mutation may be in one or more HVRs or flanking amino acids. Thus the modified and parent hexon proteins typically have identical scaffold sequences and differ only in one or more HVR. It will be appreciated, however, that each may have mutations relative to wild type hexon proteins.

For example it may have one or more mutations or modifications in one or more of HVRs 3, 5 and 7.

Nevertheless, the parent hexon protein may be a wild type hexon protein, for example of Ad2 or Ad5 serotype.

For example the modified hexon protein may contain one or more mutations (e.g. substitution, deletion or addition) relative to the parent at residues corresponding to Thr268, Thr269 and/or Glu270 of Ad5 hexon protein [shown as Thr269, Thr270 and Glu271 in the sequence provided below having accession nos. AAW65514.1; GI:58177707]. These are located in HVR5. It may contain one or more mutations at residues corresponding to Ile420, Asn421, Thr422, Glu423, Thr424, Leu425, Asp447 or Lys448 of Ad5 hexon protein [shown as Ile421, Asn422, Thr423, Glu424, Thr425, Leu426, Asp448 and Lys449 in the sequence provided below having accession nos. AAW65514.1; GI:58177707]. These are located in HVR7. Additionally or alternatively it may contain one or more mutations at residues corresponding to Glu450, Arg452 and Val453 of Ad5 hexon protein [shown as Glu451, Arg453 and Val454 in the sequence provided below having accession nos. AAW65514.1; GI:58177707]. These are located in scaffold sequence flanking HVR7. Additionally or alternatively it may comprise one or more mutations at residues corresponding to Glu212, Thr213, Glu214, Ile215, Asn216, Ser267, Met314, Asn421, Thr426, Ser446 or Asn449 of Ad5 hexon protein [shown as Glu213, Thr214, Glu215, Ile216, Asn217, Ser268, Met315, Asn422, Thr427, Ser447 and Asn450 in the sequence provided below having accession nos. AAW65514.1; GI:58177707]. Any mutation which modulates, especially reduces, binding to FX may be used. For example, a parent or wild type residue may be replaced by the residue occurring at the corresponding position in an adenovirus from a serotype with a lower affinity for FX or a lower capacity to transduce hepatocytes or splenocytes. For example, a residue in Ad5 may be exchanged for a residue from one of Ad17, Ad20, Ad25, Ad26, Ad28, Ad29, Ad44 or Ad48.

It may contain mutations at residues corresponding to one or both of Thr269 and Glu270 of Ad5 [shown as Thr270 and Glu271 in the sequence having accession nos. AAW65514.1; GI:58177707]. It may be desirable to introduce Pro or Asp at a position corresponding to Thr269. It may be desirable to introduce a Gly or Ser residue at a position corresponding to Glu270. Thus, for example, it may contain substitutions corresponding to Thr269Pro and/or Glu270Gly in Ad5.

It may contain mutations at residues corresponding to one, two three or all four of Ile420, Thr422. Glu423 and Leu425 [shown as Ile421, Thr423. Glu424 and Leu426 in the sequence having accession nos. AAW65514.1; GI:58177707]. It may be desirable to introduce Gly at a position corresponding to Ile420. Additionally or alternatively, it may be desirable to introduce Asn at a position corresponding to Thr422. Additionally or alternatively, it may be desirable to introduce Ser or Ala at a position corresponding to Glu423. Additionally or alternatively, it may be desirable to introduce Tyr at a position corresponding to Leu425. For example it may contain substitutions corresponding to one, two, three or all four of the substitutions Ile420Gly, Thr422Asn. Glu423Ser and Leu425Tyr. It may also be desirable to introduce Val or Ala at a position corresponding to Thr424 of Ad5.

A particularly desirable position to introduce a mutation may be a position corresponding to Glu450 of Ad5 [shown as Glu451 in the sequence provided below having accession nos. AAW65514.1; GI:58177707]. This is located in the final scaffold region of the protein, 6 residues downstream of HVR7. It may be desirable to introduce a neutral or positively charged residue at that position. In particular it may be desirable to replace a Glu residue occurring naturally at such a position with another residue, preferably a neutral or positively charged residue, for example Gln, Ile, Leu, Val, Arg or Lys, e.g. Gln or Arg.

The modified hexon protein may have an HVR amino acid sequence not occurring in nature.

Alternatively the modified hexon protein may be a chimeric hexon protein, comprising a scaffold from a first wild type hexon protein and at least one HVR from a second wild type hexon protein.

The chimeric hexon protein may have a scaffold region from a first adenoviral serotype and at least one HVR from a second adenoviral serotype.

For example, one or more of HVRs 3, 5 and 7 may be derived from a different serotype than the scaffold. The HVRs may be derived from a hexon protein from a serotype having lower hepatic tropism than the serotype from which the scaffold is derived.

The first serotype/wild type may be Ad2 or Ad5. The second serotype/wild type may be Ad17, Ad20, Ad25, Ad26, Ad28, Ad29, Ad44 or Ad48, or another group D serotype such as Ad9, Ad10, Ad15, Ad19, Ad22, Ad24, Ad27, Ad30, Ad32, Ad33, Ad36, Ad38, Ad39, Ad42, Ad43, Ad45, Ad47 or Ad51.

Thus it may include a scaffold region from Ad2 or Ad5, and one or more HVRs from at least one of Ad17, Ad20, Ad25, Ad26, Ad28, Ad29, Ad44 or Ad48. In some embodiments, HVR3, HVR5 and/or HVR7 are derived from Ad17, Ad20, Ad25, Ad26, Ad28, Ad29, Ad44 or Ad48, for example from Ad26 or Ad48. They may also be derived from another group D serotype such as Ad9, Ad10, Ad15, Ad19, Ad22, Ad24, Ad27, Ad30, Ad32, Ad33, Ad36, Ad38, Ad39, Ad42, Ad43, Ad45, Ad47 or Ad51.

The modified adenovirus may be a gene delivery vector.

The invention also provides a modified adenovirus as described above for use in a method of medical treatment.

The invention also provides a modified adenovirus as described above for use in gene delivery.

The invention also provides use of a modified adenovirus as described above in the preparation of a medicament for gene delivery or gene therapy.

The invention will now be described in more detail, by way of example and not limitation, by reference to the accompanying drawings and examples.

### DESCRIPTION OF THE FIGURES

FIG. 1. Analysis of FX binding to Ad5.

(A) Domain structure of human FX. Spheres indicate calcium ions.
(B) Coagulation factors (FXa, EGF2-SP FXa) were covalently coupled to SA biosensor chip. Depicted are overlayed sensorgrams. Arrows indicate the start and end of reagent injection. RU=responsive units,
(C) Antibody HX-1 directed against FX Gla-EGF1 was pre-injected over the FX biocensor chip prior to Ad5 injection. HX-1 reduces FX-Ad5 binding (δRU). Arrows indicate the start and end of reagent injection.
(D) HepG2 cells were exposed to AdKO1 in the presence of physiological FX levels. FX was pre-incubated with antibody HX-1 or IgG isotype-matched control. Cell transduction was quantified at 72 h post-infection (*p<0.001 vs control). Errors bars represent SEM.
(E) SPR analysis of Ad5 binding to FX and FX-GD that lacks the FX Gla domain. A subtracted sensorgram (FXI used as control for non-specific binding) is shown. Arrows indicate the start and end of reagent injection.
(F) MF-1 mice were pre-treated with control peanut oil or warfarin and injected with 4×10¹¹ VP/mouse Ad5 with or without pre-injection of FX or FX-GD 30 min prior to virus injection. Quantitative ELISA for β-galactosidase shows levels in liver, spleen and lung (*p=0.03).

FIG. 2. The Ad5 hexon, not fiber, binds to FX.

(A) Representation of fiber mutants used to assess binding of FX to Ad5. Ad5-21R contains 21 shaft repeats but no knob, Ad5-7.5R contains just 7.5 shaft repeats and Ad5-ΔF contains no fiber.
(B) Viruses were injected onto biosensor chips and binding to FX assessed. Subtracted sensorgrams (FX-FXI) are shown. Arrows indicate the start and end of reagent injection.
(C) HepG2 cells exposed to each adenovirus in the presence or absence of physiological FX levels. Cell bound adenovirus quantified. Error bars represent SEM.
(D) Purified Ad5 hexon protein was immobilized and binding to FX was analysed. Sensorgrams are shown for injections at 7 different concentrations of FX (50 to 0.781 μg/ml in a 2-fold dilution series). The analysis was performed in triplicate. Injection of EDTA regenerated the biosensor chip surface. Arrows indicate the start and end of reagent injection.

FIG. 3. Analysis of FX binding and adenovirus infection in an HVR-modified Ad5 vector.

(A) Sequence alignment of amino acids of Ad hexon HVRs from Ad5 and the Ad5HVR48 mutant. White on black identical residues, black on grey similar residues and black on white non-identical residues. The HVRs (1-7) are indicated.
(B) Ad5 binding to FX is abolished by replacement of Ad5 HVRs with those from Ad48, analysed by SPR (subtracted FX-FXI sensorgrams). Arrows indicate the start and end of reagent injection.
(C) SKOV3 cells were exposed to each adenovirus as indicated in the presence or absence of physiological FX levels for 1 h at 4° C. DNA was extracted and cell bound adenovirus quantified. Error bars represent SEM. (p<0.05 vs absence of FX).
(D) SKOV3 cells were exposed to each adenovirus as indicated in the presence or absence of physiological FX levels for 3 h at 37° C. Transgene expression was quantified at 48 post-infection. Error bars represent SEM. (p<0.05 vs absence of FX).
(E) MF-1 mice were injected via the intravascular route with Ad5 or Ad5HVR48 at 5×10⁹, 2×10¹⁰ or 5×10¹⁰ or VP/mouse and luciferase imaged at 48 h and quantified as photon flux. Intramuscular injections are shown as a comparison. Error bars represent SEM. *p=0.00018, **p=0.00002, ***p=0.01368 vs Ad5.

FIG. 4. Gene transfer of liver by the FX-Ad5 complex is mediated through an exosite in FX.

(A) Subtracted SPR sensorgrams (FX-FXI) showing NAPc2 or Ixolaris binding to FX without inhibition of subsequent FX-Ad5 binding. Arrows indicate the start and end of reagent injection.
(B) HepG2 cells were exposed to AdKO1 in the presence or absence of physiological FX±NAPc2 or Ixolaris at increasing concentrations. Transduction was quantified at 72 h post-infection. Error bars represent SEM. ***p<0.01 vs AdKO1+FX.
(C) MF-1 mice were pre-treated with control peanut oil or warfarin and injected with 4×10¹¹ VP/mouse Ad5 with or without pre-injection of FX alone or pre-incubated with 3 fold-molar excess of either NAPc2 or Ixolaris. Quantitative ELISA for β-galactosidase levels in liver was performed 48 h later and compared to levels in the liver achieved by FX infusion alone. Error bars represent SEM. *p<0.05 vs control.

FIG. 5. Pharmacological blockade of Ad5 binding to FX by snake venom protein X-bp blocks liver transduction in vivo.

(A) Subtracted SPR sensorgram (FX-FXI) shows X-bp binding with high affinity (increase in RU following X-bp injection) and ablates subsequent FX-Ad5 binding (no change in RU following Ad5 injection). Arrows indicate the start and end of reagent injection.
(B) HepG2 cells were exposed to AdKO1 in the presence of FX alone or FX pre-incubated with X-bp at different FX:X-bp molar ratios (as shown). ***p<0.0001 vs no X-bp. Error bars represent SEM.
(C) MF-1 mice were pre-treated with control peanut oil or warfarin and injected with 4×10¹¹ VP/mouse Ad5 with or without pre-injection of FX alone or pre-incubated with 3 fold-molar excess of X-bp. *p=0.006; **p=0.0002. Error bars represent SEM.
(D) MF-1 mice (non-warfarinised) were injection with X-bp 30 min prior to Ad5 injection. 48 later liver gene transfer was quantified by ELISA (bar graph) and visualized by staining for β-galactosidase (not shown). (**p=0.0002) Error bars represent SEM.

FIG. 6. FX binding to alternate human adenovirus serotypes.

(A) Phylogenetic tree based on alignment of hexon HVR amino acid sequences by ClustalW and visualized by Treeview (http://taxonomy.zoology.gla.ac.uk/rod/treeview.html) showing FX binders (“+”, “++” or “+++”) and non-binders (“−”) and strength of FX binding determined by SPR is indicated. Ad viruses not tested by SPR are shown in grey.
(B) Representative SPR sensograms depict human adenovirus serotypes that show strong (++ or +++), weak (+) or no binding to FX. Insets show expanded y-axis to show weak (Ad35) or not detectable binding (Ad26 and Ad48). Arrows indicate the start and end of injection.
(C) HepG2 cells were exposed to each adenovirus in the presence or absence of physiological FX. DNA was extracted and cell bound adenovirus quantified. Error bars represent SEM. (p<0.05 vs absence of FX). Images show transgene (eGFP) expression following exposure for 3 h at 37° C. in the presence/absence of FX.

FIG. 7. In vitro MDA-BB-435 cell binding and transduction mediated by Ad5, Ad5HVR48 and Ad48 in the presence and absence of FX. (A) Cells were exposed to 1000 VP/cell at 4oC for 1 h, harvested and DNA extracted for Taqman analysis and genome quantification. (B) Cells were exposed to 5000 VP/cell at 37oC for 3 h, washed and harvested at 48 h postinfection. Transgene expression was quantified from cell harvested and normalised to protein content. *p<0.05 vs. in the absence of FX. NS=not significantly different.

FIG. 8. The role of the serine protease domain of FX in cell binding. HepG2 cells were incubated at 4° C. in serum free media for 1 h in the presence of Ad5 (1000 VP/cell) and either FX or FX pre-incubated with rNAPc2 or tick anticoagulant peptide (TAP; as a control). Cell association of virus was quantified by real time PCR. NS=not significantly different.

FIG. 9. In vivo assessment of coagulation factor rescue of liver transduction by Ad5. To assess the ability of coagulation factors to “rescue” liver transduction in mice, animals (MF1 outbred mice, 6-12 weeks of age, approximately 30 g) were administered subcutaneously with 133 mg/mouse warfarin (Sigma-Aldrich, Dorset, UK) (suspended in peanut oil) 3 days and 1 day prior to adenovirus injection. Thirty minutes prior to virus injection, mice were injected with coagulation factors. Based on the physiological concentrations of factors VII, IX, X, XI and Protein C (10, 90, 170, 30 and 60 nM, respectively) and the circulating plasma volume of a mouse (approximately 2 mls), mice were dosed with sufficient coagulation factor (1.25, 10, 20, 9.6 and 7.2 microgrammes/mouse, respectively) to reconstitute to physiological levels. Injection of 4×1011 VP/mouse virus was performed intravenously. At sacrifice (48 h), organs were harvested and the levels of β-galactosidase activity were quantified by commercial ELISA (Roche Diagnostics, Mannheim, Germany).

FIG. 10. FVII, FIX, FX, FXI and PC binding to Ad5 hexon. Purified Ad5 hexon (521 RU) was immobilized on a CM5 biosensor chip by amine coupling according to the manufacturer's instructions (Biacore). Binding of coagulation factors FX (A), FVII (B), PC(C), FIX (D) and FXI (E) was analysed by SPR. Subtracted sensorgrams against control are shown for injections at 11 different concentrations of coagulation factor in triplicate (50-0.05 μg/ml in a 2-fold dilution series). The sensorchip was regenerated by injection of 5 mM EDTA.

FIG. 11. The effect of X-bp on liver targeting of Ad5 in rats. Wistar Kyoto rats were either injected with saline or X-bp at a 3-fold molar excess of X-bp:FX (=1.1 μg/g X-bp) 30 mins prior to injection of Ad5 (1×1011 VP/rat). 5 days post-injection livers were harvested and β-galactosidase quantified or visualised en face using X-gal. *p<0.0001 vs Ad5.

FIG. 12. Alignment of HVRs and flanking scaffold sequences of hexon proteins from different human adenoviral serotypes.

FIG. 13. Alignment of FX sequence from various species.

FIG. 14. Adenoviral hexon cloning strategy showing intermediate and shuttle plasmids for homologous recombination.

FIG. 15. Analysis of β-Galactosidase transgene expression in HeLa cells infected with control Ad5CMVlacZ and different hexon HVR swaps mutant adenoviral vectors. Values were analyzed 48 hours post-transfection.

FIG. 16. Analysis of binding capacity of control Ad5CMVlacZ and different HVR-hexon mutant adenoviruses in SKOV3 cells. Fold increase relative to vehicle control is represented.

FIG. 17. Analysis of β-Galactosidase transgene expression in SKOV3 cells infected with control Ad5CMVlacZ and different HVR-hexon mutant adenoviral vectors. * p<0.05 Vs virus alone.

FIG. 18. Analysis of β-Galactosidase transgene expression in SKOV3 cells infected with control Ad5CMVlacZ and different HVR-hexon mutant adenoviral vectors. Fold increase relative to vehicle control is represented.

FIG. 19. Analysis of β-Galactosidase transgene expression in SKOV3 cells infected with control Ad5CMVlacZ and different HVR-hexon mutant adenoviral vectors. Fold increase relative to vehicle control is represented.

FIG. 20. Analysis of β-Galactosidase transgene expression in HeLa cells infected with control Ad5CMVlacZ and different hexon point mutant adenoviral vectors. Values were analyzed 48 hours post-transfection.

FIG. 21. Analysis of binding capacity of control Ad5CMVlacZ and different point mutant adenoviruses in SKOV3 cells.

FIG. 22. Analysis of β-Galactosidase transgene expression in SKOV3 cells infected with control Ad5CMVlacZ and different point mutant hexon adenoviral vectors.

FIG. 23. Analysis of β-Galactosidase transgene expression in SKOV3 cells infected with control Ad5CMVlacZ and different point mutant-hexon adenoviral vectors. Fold increase relative to vehicle control is represented.

FIG. 24. Analysis of β-Galactosidase transgene expression in 200 μg of liver protein from mice injected with Ad5CMVlacZ or point mutant hexon adenoviruses.

FIG. 25. Analysis of FX-adenovirus particle interaction by Surface Plasmon Resonance.

FIG. 26. Contact points between Ad5 hexon and FX. Amino acid sequence from human adenovirus serotypes 5, 35, 50, 49 and 26 are represented. Boxes indicate hyper variable regions and arrows indicate putative FX binding sites.

### DETAILED DESCRIPTION OF THE INVENTION

### Adenoviruses

Adenoviruses are non-enveloped viruses containing a linear double-stranded DNA genome which infect various mammalian species including humans.

The genome is typically approximately 30-38 kp in length, and encodes a number of genes including so-called Early (E1a, E1b, E2a, E2b, E3, E4) and Late (L1, L2, L3, L4, L5) genes, flanked by 5′ and 3′ inverted terminal repeats (ITRs). It also contains a packaging signal.

The genome is enclosed in a capsid made up of three major proteins, namely penton, hexon, and fiber.

Adenoviruses are frequently used as gene transfer vectors to deliver heterologous (i.e. non-adenoviral) DNA to a target cell or tissue. The heterologous DNA normally comprises or consists of a heterologous gene (often referred to as a transgene).

Such vectors lack one or more genes essential for viral replication. The viral gene is typically deleted from the genome and replaced by the heterologous gene or genes. These vectors are thus replication-defective and are not capable of productive infection resulting in generation of viral progeny which are identical to the parent (unless the same cell is also infected with a helper virus capable of complementing the deficiency present in the vector genome).

Three generations of adenoviral vectors have been generated to date. The first generation lacked the E1 gene. The second generation combined deletion of E1 and/or E3 with deletions of E2 and/or E4. The third generation retains only the ITRs and packaging signal with the rest of the genome replaced by heterologous DNA, and are often called “gutless” or “gutted” vectors, or “helper-dependent adenoviruses” since they rely on a helper adenovirus to supply all viral proteins. See Alba et al. (2005) for review.

The term “adenovirus” as used in this specification is intended to encompass such replication-defective vectors as well as replication-competent viruses. The term “transduction” is used in this specification to refer to the process of introduction of a heterologous gene to a target cell or tissue by a gene transfer vector.

In vitro, adenoviruses infect hepatocytes via the coxsackie and adenovirus receptor (CAR). However this route of infection is not significant in vivo. A number of mutations are known which reduce or ablate infection via the CAR; examples are described in De Geest et al, 2005, O'Riordan et al, 1999, Niu et al., 2007, Lanciotti et al., 2003. For in vitro studies of adenoviral infection or transduction of hepatocytes via the mechanism involving hexon protein and FX, it may be desirable to use viruses comprising such a mutation. Additionally or alternatively it may be desirable to utilise a cell type for transduction in vitro which does not express CAR or expresses it at only low levels. An example is SKOV3 cells (Kim et al., 2002.)

### Adenoviral Hexon Proteins

Hexon proteins provide the major source of antigenicity in adenoviruses. There are at least 49 serotypes known in humans (see FIG. 6A). The hexon proteins of different serotypes comprise a scaffold of sequence which is relatively conserved between serotypes and which forms the major structural fragment of the hexon protein, and seven highly variable surface loops, referred to as Hypervariable Regions (HVRs). These are illustrated in FIG. 12.

Thus in Ad5, HVR1 has the sequence EAATALEINLEEEDDDNEDEVDEQAEQQKTHVEGQAPYSGINITK, HVR2 has the sequence GVEGQTP, HVR3 has the sequence QWYETEINH, HVR4 has the sequence GILVKQQNGKLES, HVR5 has the sequence FSTTEATAGNGDNLTP, HVR6 has the sequence PTIKEGNSRELM and HVR7 has the sequence INTETLTKVKPKTGQENGWEKDATE.

In Ad26, HVR1 has the sequence TKEKQGTTGGVQQEKDVTKTFGVAATGGINITN, HVR2 has the sequence GTDETAENGKKD, HVR3 has the sequence NWQENEA, HVR4 has the sequence AKFKPVNEGEQPKD, HVR5 has the sequence LDIDFAYFDVPGGSPPAGGSGEEYKA, HVR6 has the sequence PGTSDNSSEINL and HVR7 has the sequence GTNSTYQGVKITNGNDGAEESEWEKDDA.

In Ad48, HVR1 has the sequence EKKNGGGSDANQMQTHTFGVAAMGGIEITA, HVR2 has the sequence GIDATKEEDNGKE, HVR3 has the sequence NWQDSDN, HVR4 has the sequence AKFKTPEKEGEEPKE, HVR5 has the sequence FDIPSTGTGGNGTNVNFKP, HVR6 has the sequence PGKEDASSESNL and HVR7 has the sequence GTNAVYQGVKVKTTNNTEWEKDTA.

Corresponding HVR5 in other serotypes can be identified by individual alignment with the Ad5 sequence, or directly from FIG. 12.

When a given hexon protein is aligned with a reference hexon protein to yield maximum sequence identity, it is possible to identify scaffold sequences and HVRs as shown in FIG. 12. The reference hexon protein may be any of the hexon proteins shown in FIG. 12, e.g. Ad5. Thus a full length hexon protein has 7 HVRs and 8 scaffold regions. A hexon protein for use in the present invention preferably has scaffold regions which have at least 60%, such as at least 65%, at least 70%, at least 75%, at least 80%, at least 85%, at least 90% or at least 95% identity with the corresponding scaffold regions of at least one of the hexon proteins whose sequence is shown in FIG. 12, for example Ad5. The full-length sequence of Ad5 hexon protein is:

| (Accession no. AAW65514.1; GI: 58177707) | |
|---|---|
| 1 | MATPSMMPQW SYMHISGQDA SEYLSPGLVQ FARATETYFS LNNKFRNPTV APTHDVTTDR |
| 61 | SQRLTLRFIP VDREDTAYSY KARFTLAVGD NRVLDMASTY FDIRGVLDRG PTFKPYSGTA |
| 121 | YNALAPKGAP NPCEWDEAAT ALEINLEEED DDNEDEVDEQ AEQQKTHVFG QAPYSGINIT |
| 181 | KEGIQIGVEG QTPKYADKTF QPEPQIGESQ WYETEINHAA GRVLKKTTPM KPCYGSYAKP |
| 241 | TNENGGQGIL VKQQNGKLES QVEMQFFSTT EAAAGNGDNL TPKVVLYSED VDIETPDTHI |
| 301 | SYMPTIKEGN SRELMGQQSM PNRPNYIAFR DNFIGLMYYN STGNMGVLAG QASQLNAVVD |
| 361 | LQDRNTELSY QLLLDSIGDR TRYFSMWNQA VDSYDPDVRI IENHGTEDEL PNYCFPLGGV |
| 421 | INTETLTKVK PKTGQENGWE KDATEFSDKN EIRVGNNFAM EINLNANLWR NFLYSNIALY |
| 481 | LPDKLKYSPS NVKISDNPNT YDYMNKRVVA PGLVDCYINL GARWSLDYMD NVNPFNHHRN |
| 541 | AGLRYRSMLL GNGRYVPFHI QVPQKFFAIK NLLLLPGSYT YEWNFRKDVN MVLQSSLGND |
| 601 | LRVDGASIKF DSICLYATFF PMAHNTASTL EAMLRNDTND QSFNDYLSAA NMLYPIPANA |
| 661 | TNVPISIPSR NWAAFRGWAF TRLKTKETPS LGSGYDPYYT YSGSIPYLDG TFYLNHTFKK |
| 721 | VAITFDSSVS WPGNDRLLTP NEFEIKRSVD GEGYNVAQCN MTKDWFLVQM LANYNIGYQG |
| 781 | FYIPESYKDR MYSFFRNFQP MSRQVVDDTK YKDYQQVGIL HQHNNSGFVG YLAPTMREGQ |
| 841 | AYPANFPYPL IGKTAVDSIT QKKFLCDRTL WRIPFSSNFM SMGALTDLGQ NLLYANSAHA |
| 901 | LDMTFEVDPM DEPTLLYVLF EVFDVVRVHQ PHRGVIETVY LRTPFSAGNA TT |

Amino acid positions in the Ad5 hexon protein may be numbered as shown in this sequence. However, alternative numbering is sometimes used in which the initiating methionine residue is not counted; the subsequent Ala residue is then designated as position 1. Thus, for example, the glutamic acid residue at position 451 in the sequence shown above can either be designated E450 or E451, depending on which numbering system is used. When considered in context, however, it is clear throughout this specification which numbering applies to any given residue.

An HVR is a variable sequence which in nature typically comprises between about 7 and about 60 amino acids. However it is believed that HVRs of different lengths may be tolerated. An HVR may, but need not, have greater than about 50%, 55%, 60%, 65%, 70%, 75%, 80%, 85%, 90% or 95% sequence identity with an HVR of a serotype shown in FIG. 12.

An HVR may be between 5 and 150 amino acids in length. Typically it will be between about 5 and about 100 amino acids in length. For example, it may be between about 7 amino acids and about 60 amino acids (by “about” is meant +/−10%).

For example, HVR1 may be between about 20 and about 60 amino acids, for example, between 21 and 54 amino acids.

HVR2 may be between about 5 and about 20 amino acids, for example between 8 and 14 amino acids.

HVR3 may be between about 5 and about 15 amino acids, for example between 7 and 9 amino acids.

HVR4 may be between about 5 and about 25 amino acids, for example between 6 and 18 amino acids.

HVR5 may be between about 5 and about 25 amino acids, for example between 9 and 20 amino acids.

HVR6 may be between about 5 and about 20 amino acids, for example between 11 and 14 amino acids.

HVR7 may be between about 15 and about 35 amino acids, for example between 21 and 29 amino acids.

In HVR11t may be desirable to have an Ala residue at a position corresponding to Ala171 of Ad5 hexon protein [shown as Ala172 in the sequence provided above having accession nos. AAW65514.1; GI:58177707]. Additionally or alternatively it may be desirable to have an Ile residue at a position corresponding to Ile178 of Ad5 hexon protein [shown as Ile179 in the sequence provided above having accession nos. AAW65514.1; GI:58177707].

In HVR51t may be desirable to have a Phe residue at a position corresponding to Phe266 of Ad5 hexon protein [shown as Phe267 in the sequence provided above having accession nos. AAW65514.1; GI:58177707]. Additionally or alternatively it may be desirable to have a Phe or Tyr residue at a position corresponding to Phe265 of Ad5 hexon protein [shown as Phe266 in the sequence provided above having accession nos. AAW65514.1; GI:58177707].

In HVR6 it may be desirable to have an Ala or Ser residue at a position corresponding to Ser310 of Ad5 hexon protein [shown as Ser311 in the sequence provided above having accession nos. AAW65514.1; GI:58177707], and/or a Ser or Asn residue at a position corresponding to Asn309 of Ad5 hexon protein [shown as Asn310 in the sequence provided above having accession nos. AAW65514.1; GI:58177707], and/or a Met or Leu residue at a position corresponding to Met314 of Ad5 hexon protein [shown as Met315 in the sequence provided above having accession nos. AAW65514.1; GI:58177707].

In HVR 7 it may be desirable to have a Trp residue at a position corresponding to Trp438 of Ad5 hexon protein [shown as Trp439 in the sequence provided above having accession nos. AAW65514.1; GI:58177707]. Additionally or alternatively it may be desirable to have a Val or Ile residue at a position corresponding to Val428 of Ad5 hexon protein [shown as Val429 in the sequence provided above having accession nos. AAW65514.1; GI:58177707].

In some embodiments, when mutations (e.g. substitutions or deletions) are made relative to a wild type or parent HVR sequence, it may be desirable that the resulting HVR differs from the wild type or parent HVR by a maximum of five residues, e.g. a maximum of four residues, a maximum of three residues, a maximum of two residues, or a maximum of one residue.

Thus, for example, a modified Ad5 hexon protein may contain an HVR3, an HVR5, and/or an HVR7 which differs from the wild type Ad5 HVR3, HVR5 and/or HVR7 sequence by a maximum of five residues, e.g. a maximum of four residues, a maximum of three residues, a maximum of two residues, or a maximum of one residue.

For example, the HVR5 sequence may differ from a wild type HVR5 sequence by a maximum of five residues, four residues, three residues, two residues, or one residue, including residues corresponding to positions 269 and/or 270 of Ad5 HVR5 [shown as Thr270 and Glu271 in the sequence provided above having accession nos. AAW65514.1; GI:58177707]. It may have the residues Pro and/or Gly at these positions. The wild type HVR sequence may be Ad5 HVR5.

The HVR7 sequence may differ from a wild type HVR7 sequence by a maximum of five residues, four residues, three residues, two residues, or one residue, including residues corresponding to one or more of positions 420, 422, 423 and 425 of Ad5 HVR7 [shown as positions 421, 423, 424 and 426 in the sequence provided above having accession nos. AAW65514.1; GI:58177707]. It may have one or more of the residues 420Gly, 422Asn, 423Ser and/or 425Tyr at these positions.

Thus the hexon protein considered as a whole may have at least 60% identity, such as at least 65%, at least 70%, at least 75%, at least 80%, at least 85%, at least 90% or at least 95% identity with the overall sequence of one of the hexon proteins in FIG. 12 (e.g. Ad5), but need not do so as long as the scaffold sequences have the required degree of identity.

Chimeric hexon proteins comprise a fragment of scaffold from a first serotype and at least one HVR from a second serotype. For example, a chimeric hexon protein may contain scaffold from Ad2 or Ad5 serotypes, e.g. Ad5 serotype, and one or more of HVRs 3, 5 and 7, especially HVR5 and/or HVR7, from another serotype. That other serotype may be (for example) Ad17, Ad20, Ad25, Ad26, Ad28, Ad29, Ad44 or Ad48, for example Ad26 or Ad48. In some embodiments, it may be desirable that only HVR5, only HVR7 or only HVR5 and HVR7 are from the other serotype, and the other HVRs are from the same serotype as the scaffold.

However both scaffold and HVR may contain modifications or mutations, e.g. substitutions, relative to the sequences shown in FIG. 12.

A hexon fragment capable of binding to FX Gla may comprise at least 20, at least 50, at least 100, at least 200, at least 300 at least 400, at least 500, at least 600, at least 700, at least 800 or at least 900 contiguous amino acids of a hexon protein as shown in FIG. 12 or a variant thereof as described above having at least 60% sequence identity over the scaffold regions with any of the sequences shown in FIG. 12. It may comprise at least one HVR, and may comprise 2, 3, 4, 5, 6 or 7 HVRs.

It will be appreciated that FIG. 12 does not show the full length sequences of either the first or final (eighth) scaffold regions of the relevant hexon proteins. It will be understood that, where a degree of sequence identity to a particular scaffold region is specified, it is intended to refer to the full length sequence of those regions, and not simply the fragments of those regions shown in FIG. 12.

### Factor X (FX)

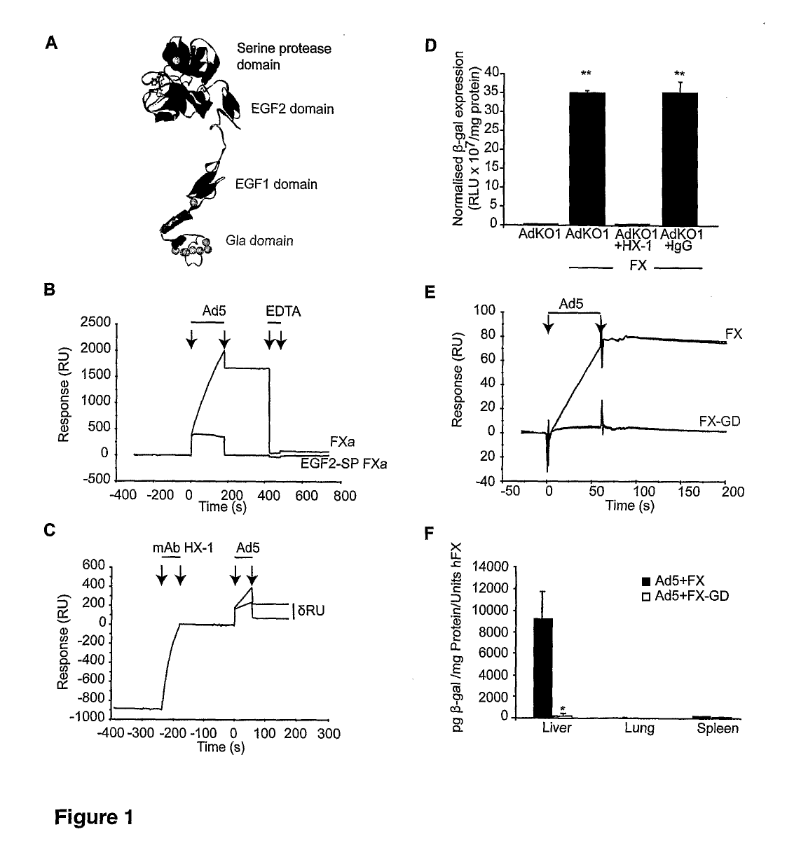

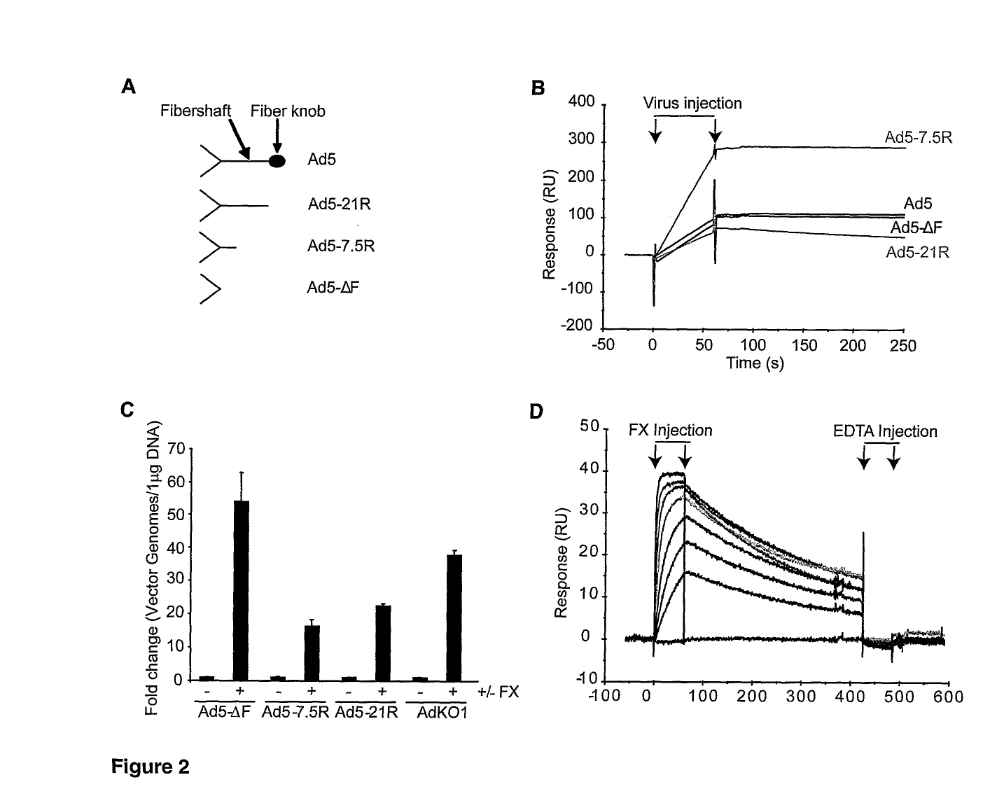

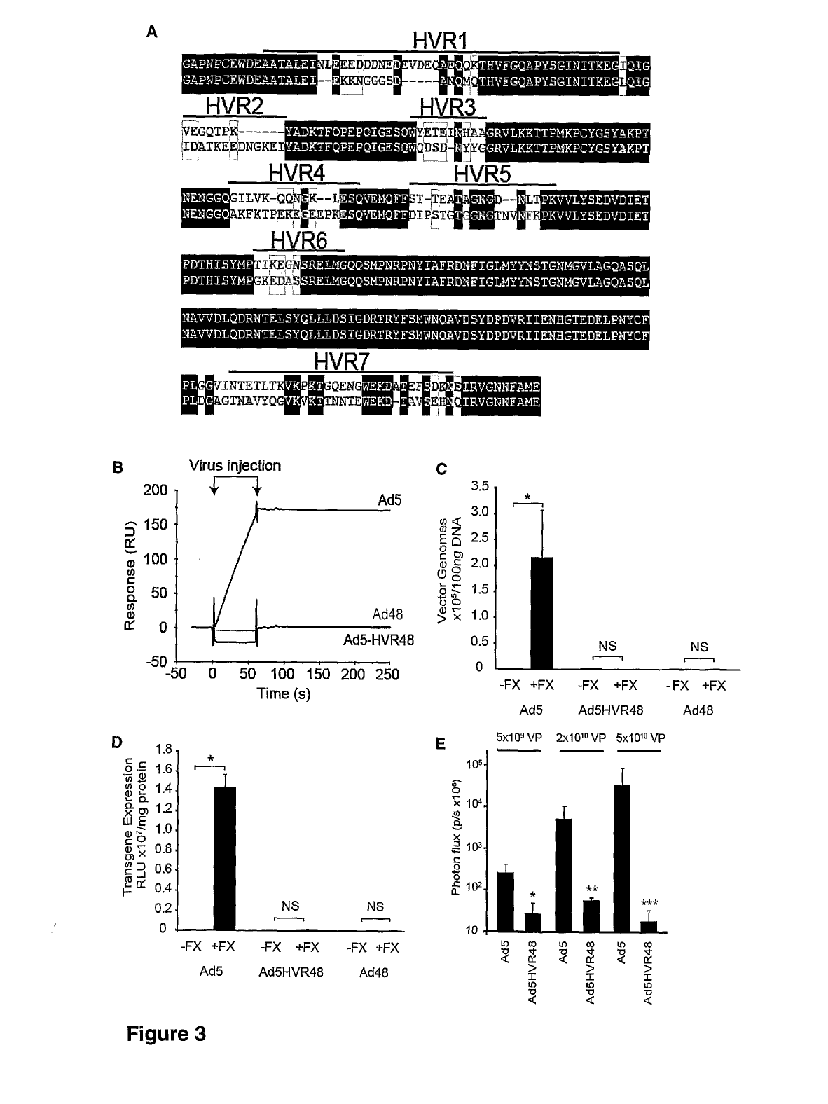

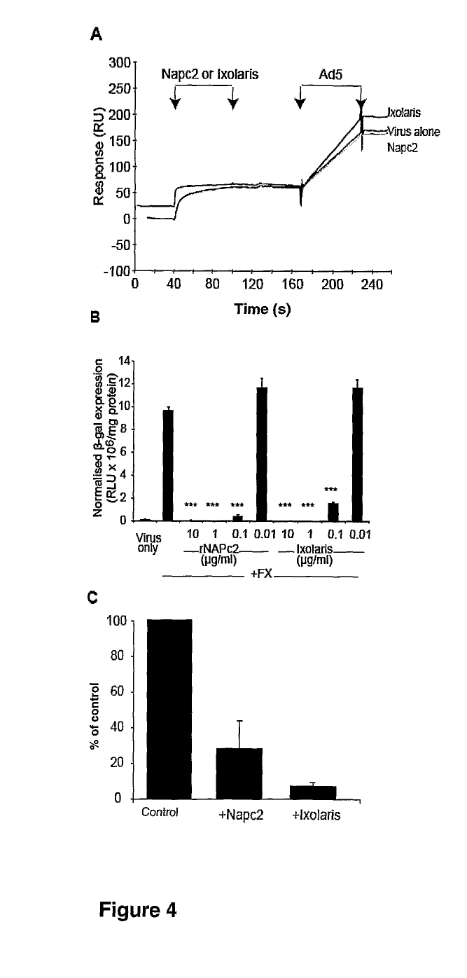

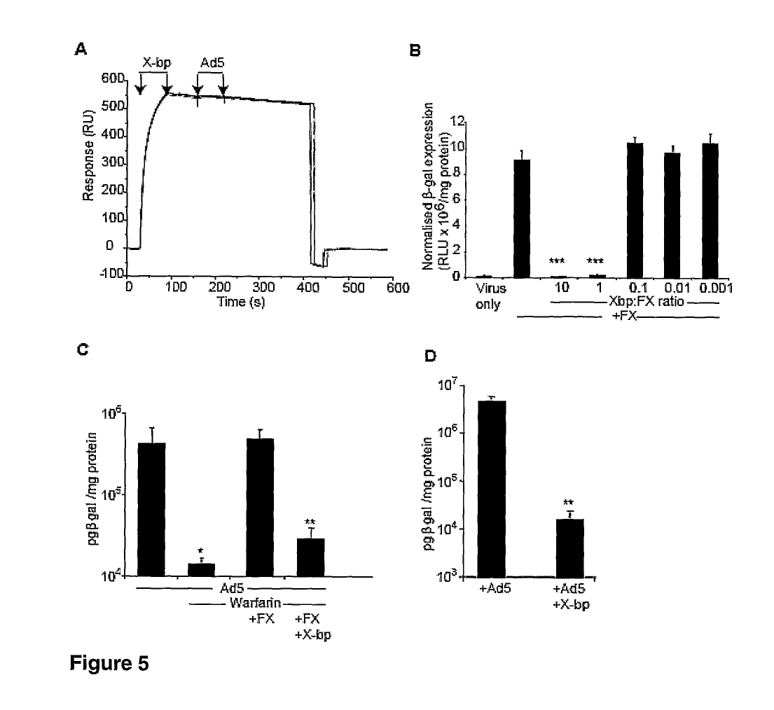

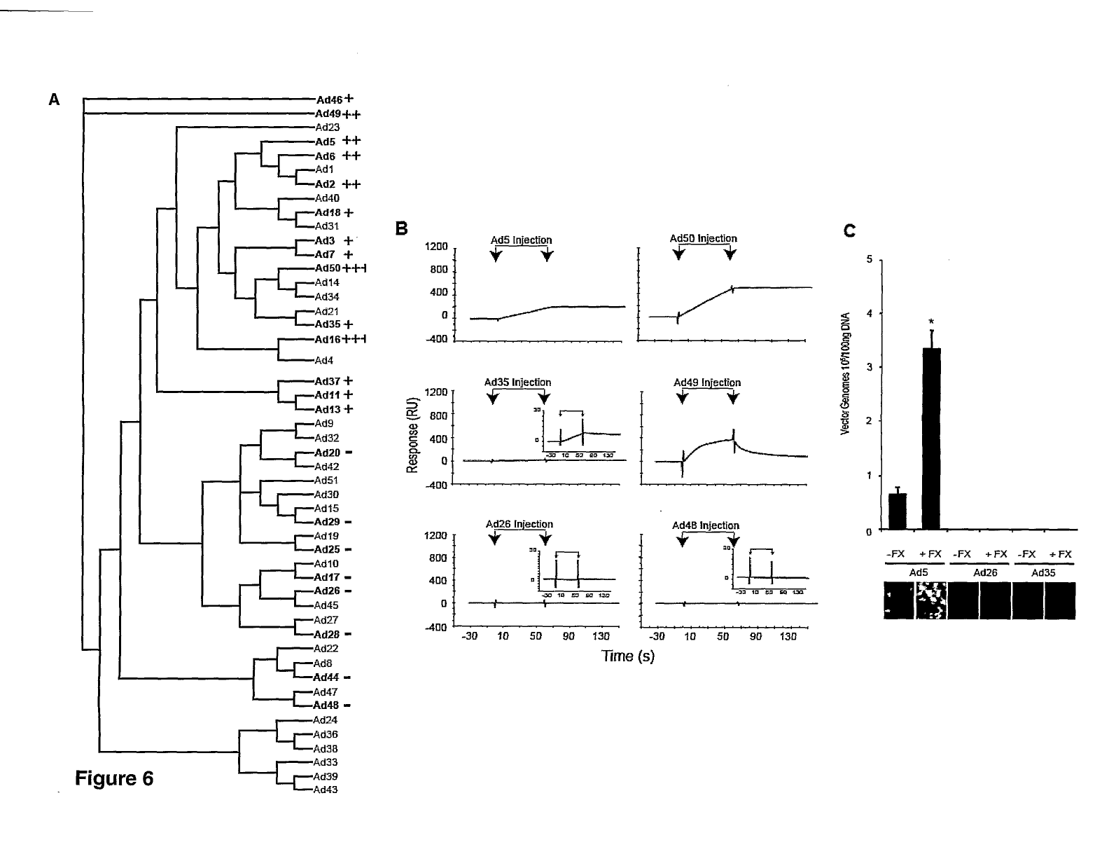

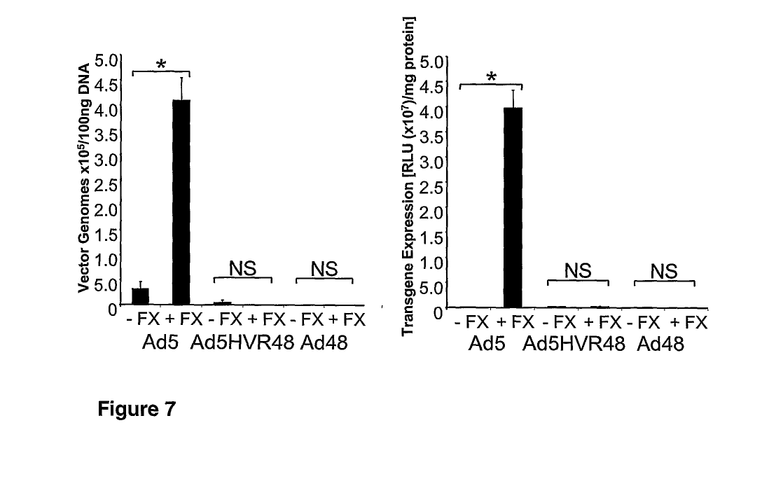

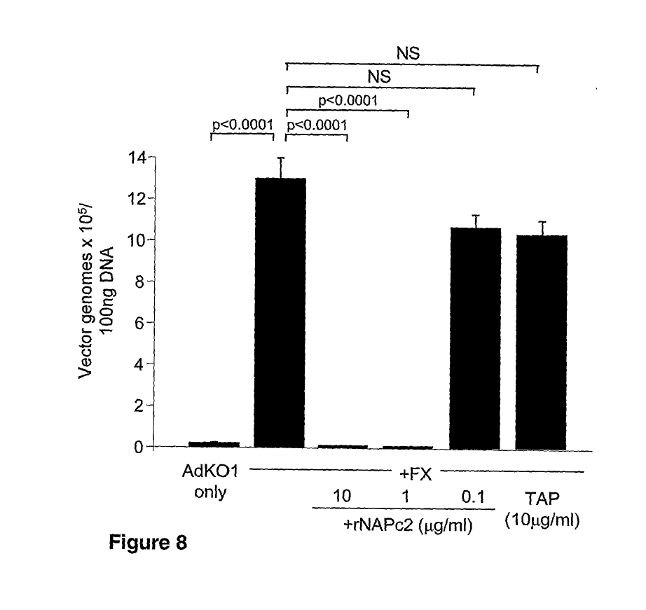

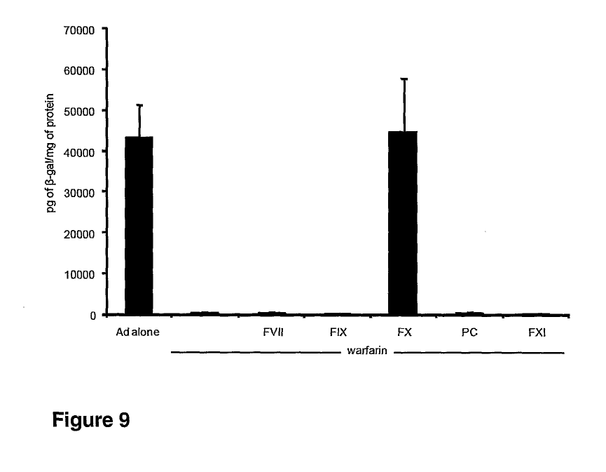

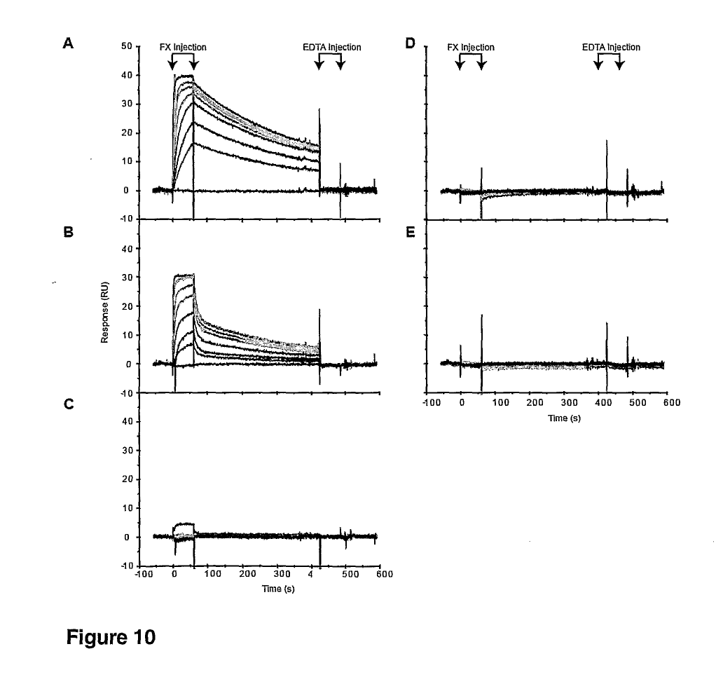

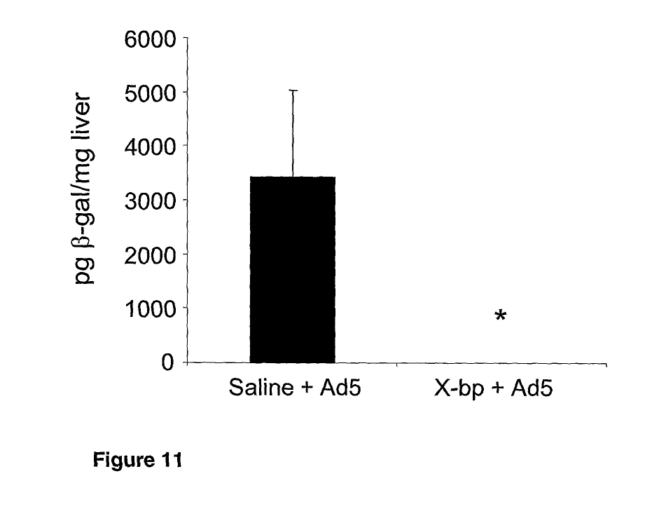

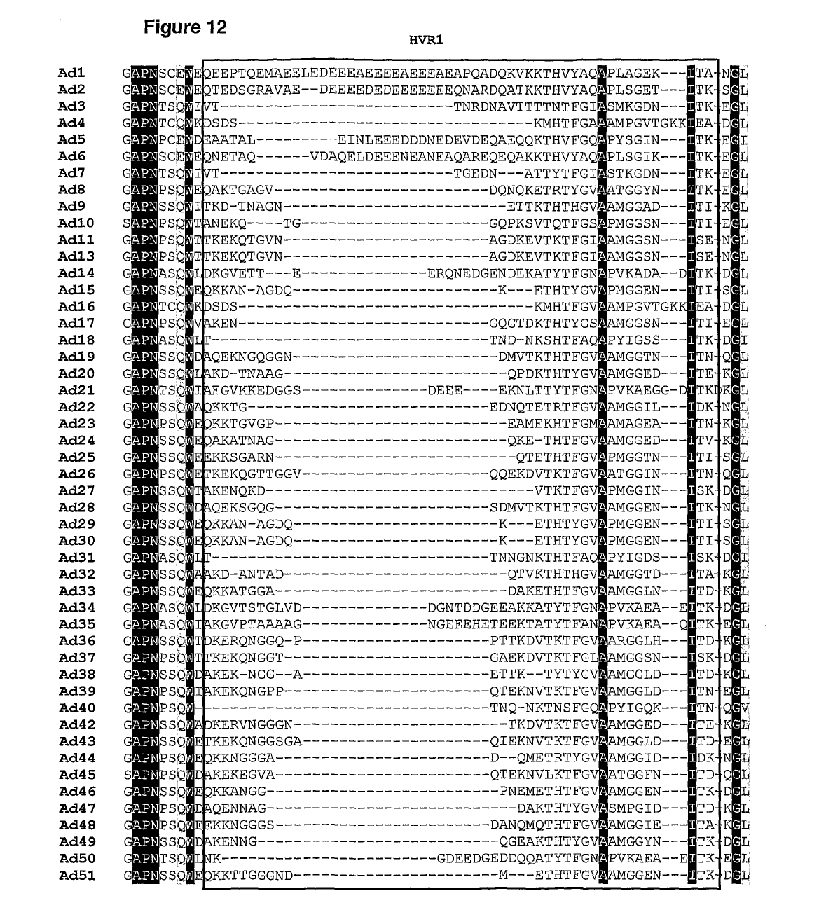

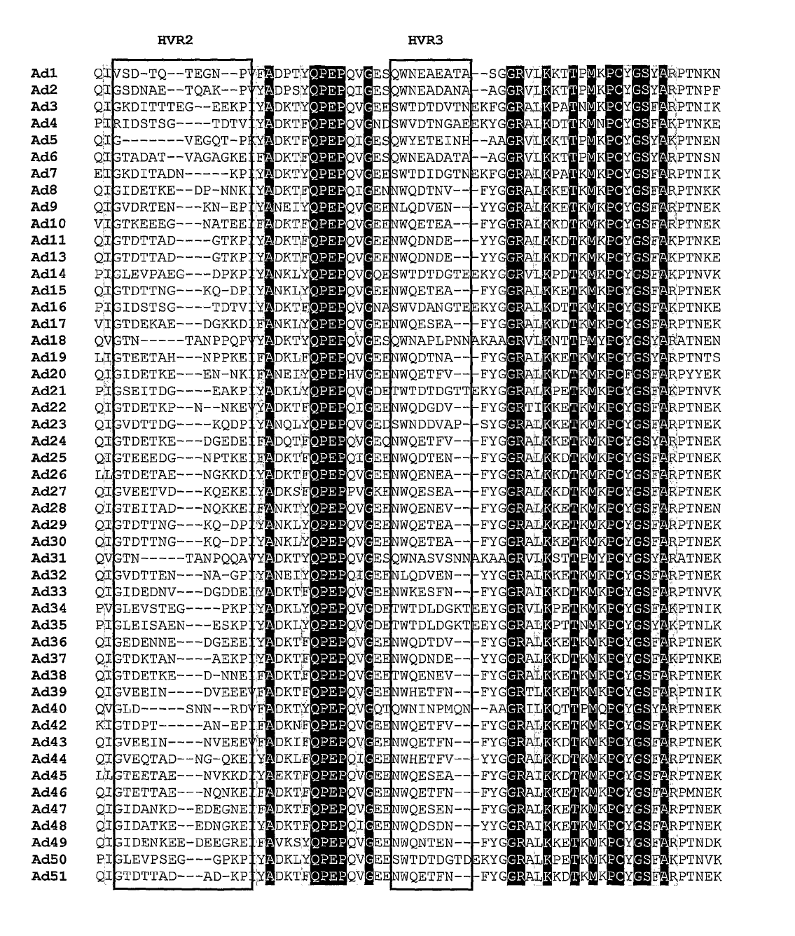

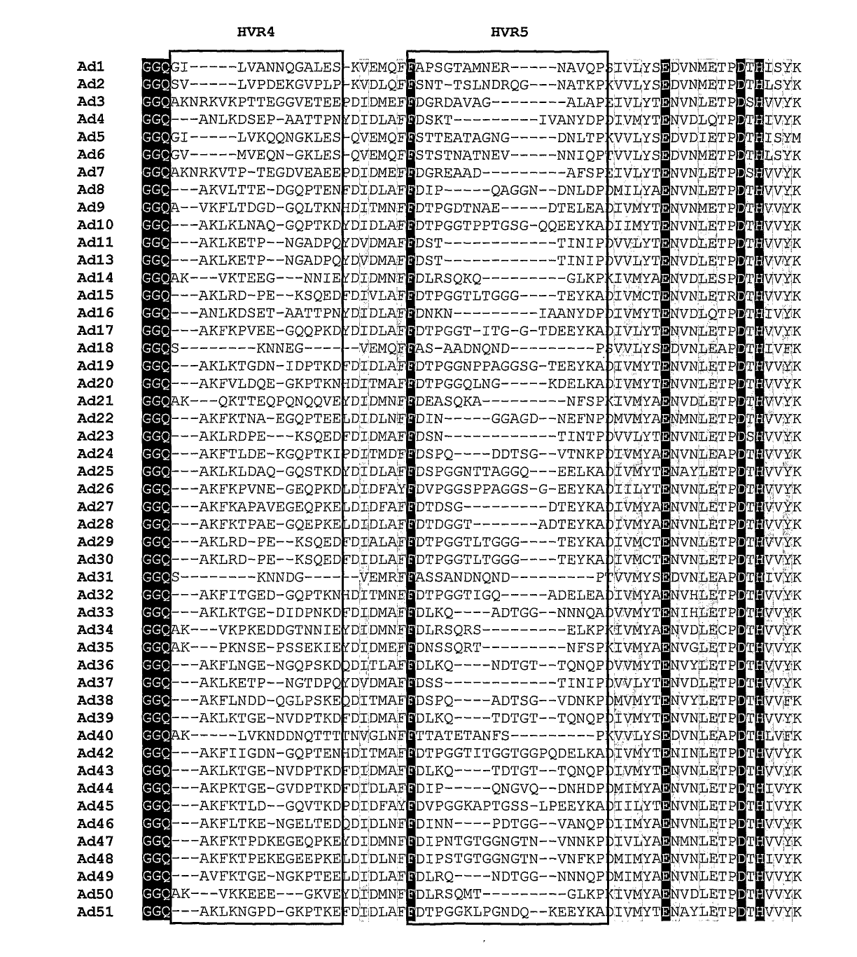

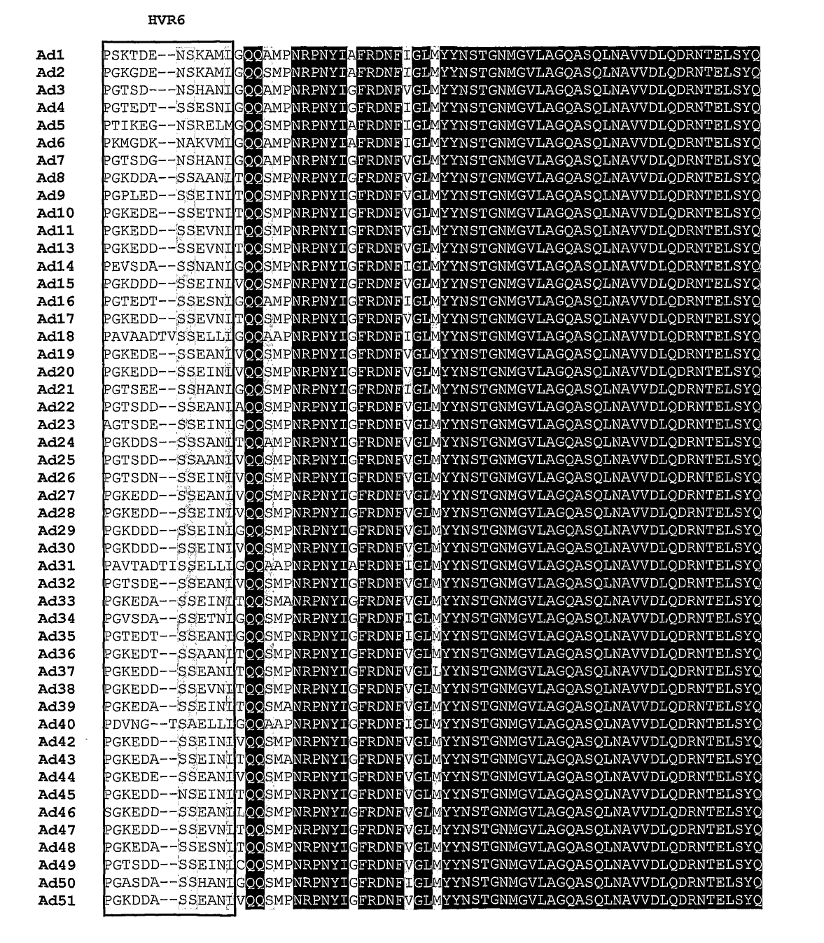

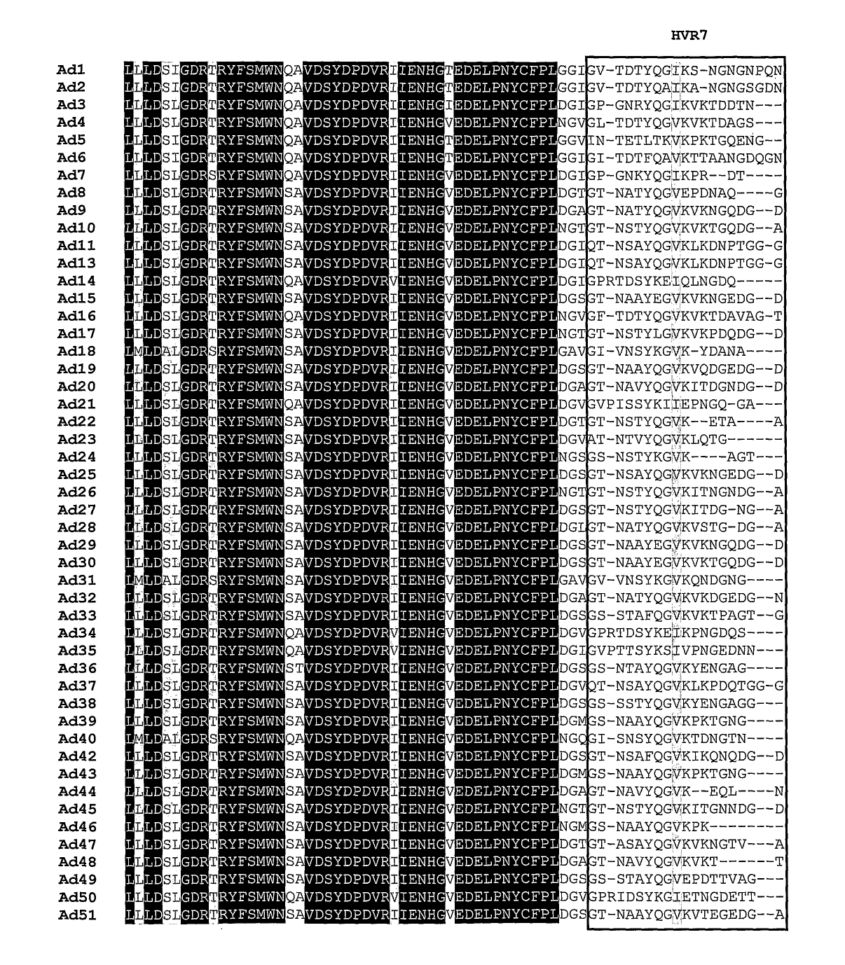

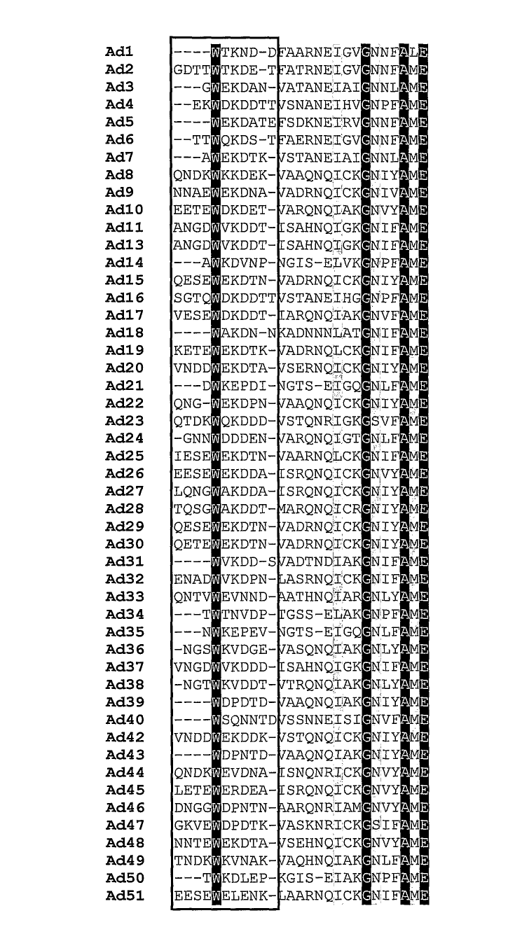

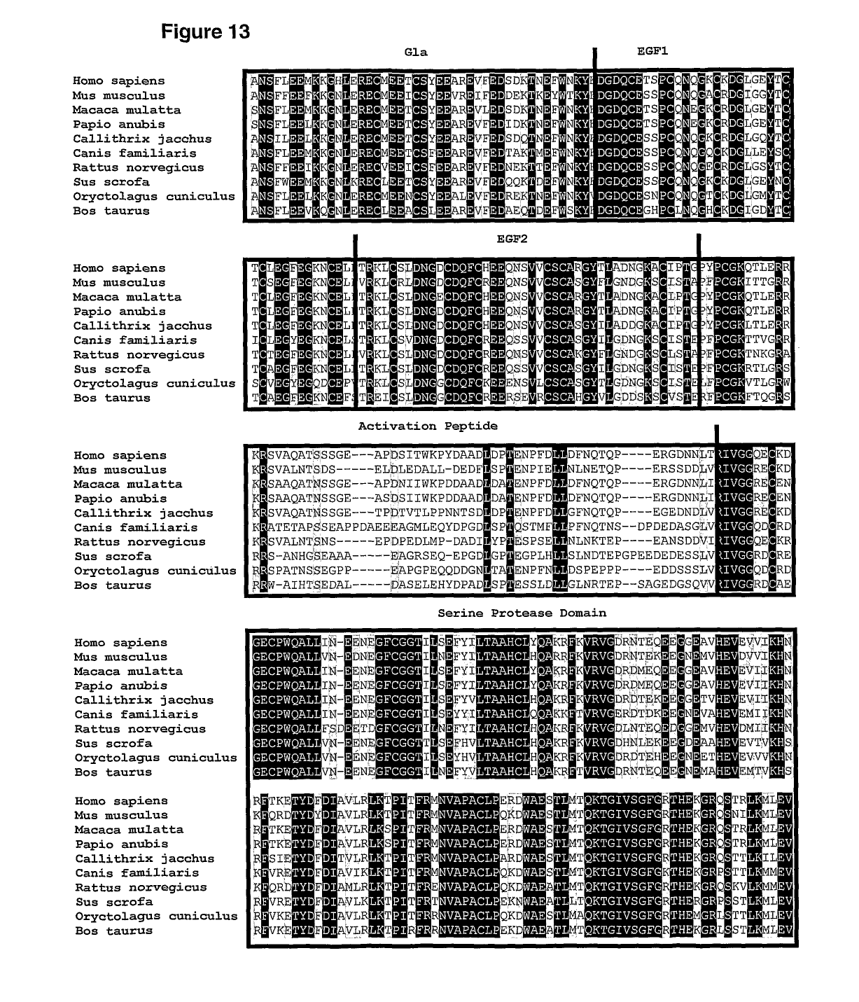

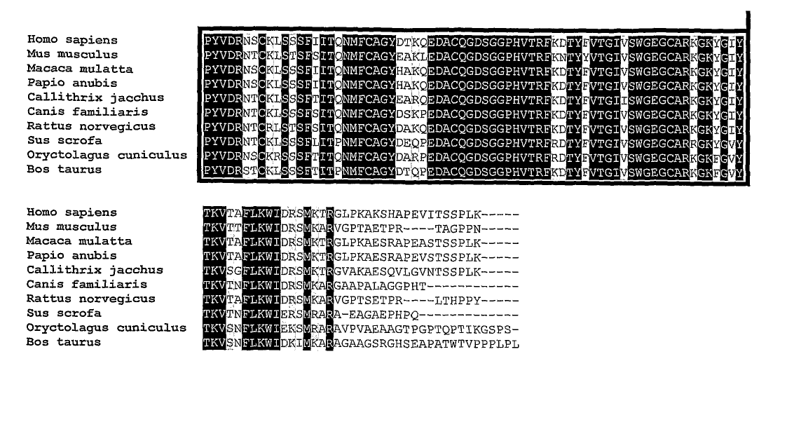

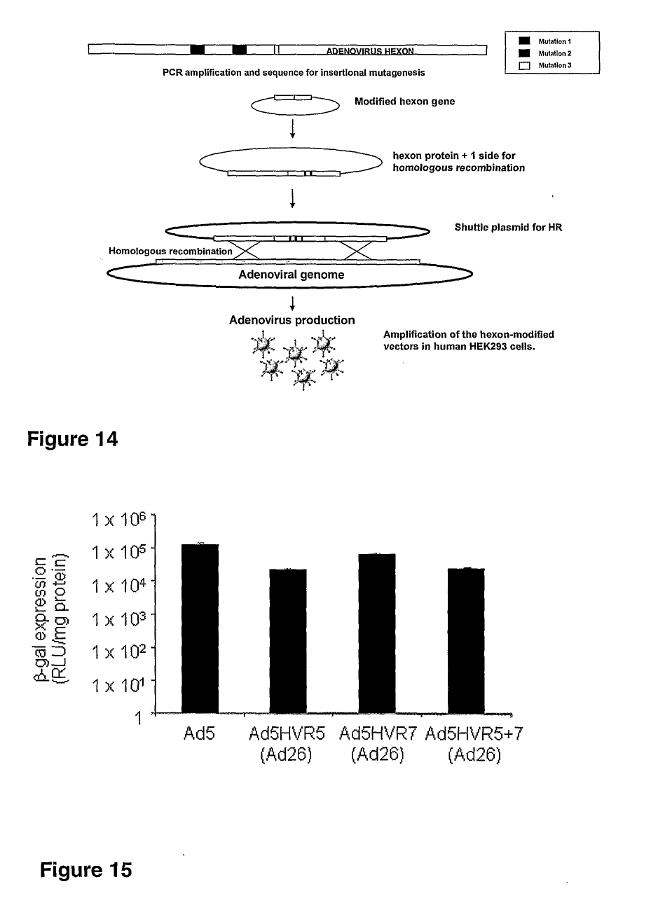

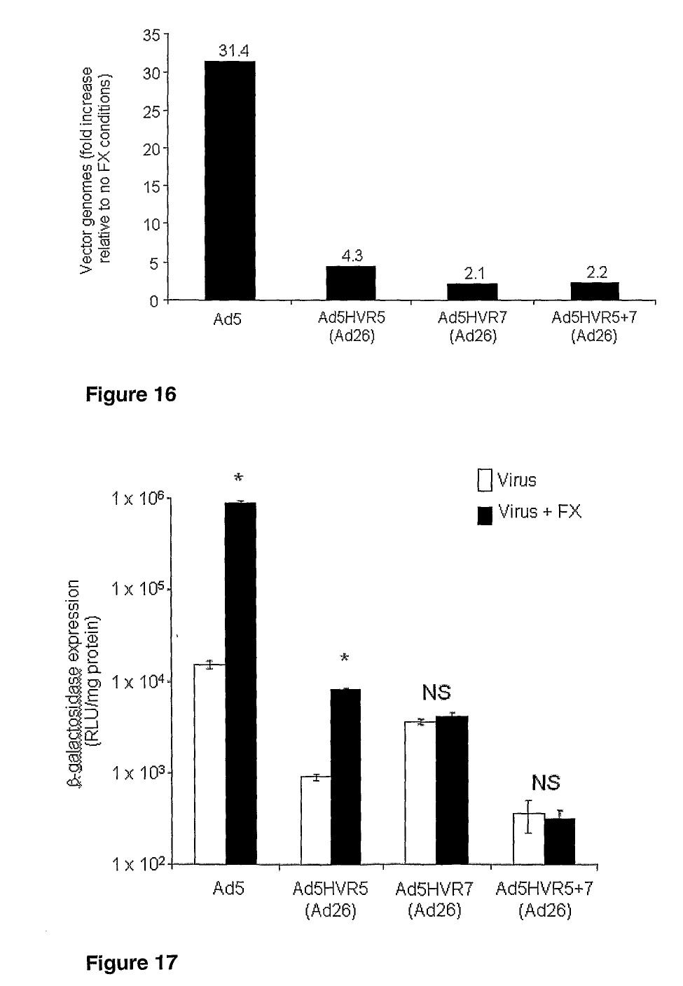

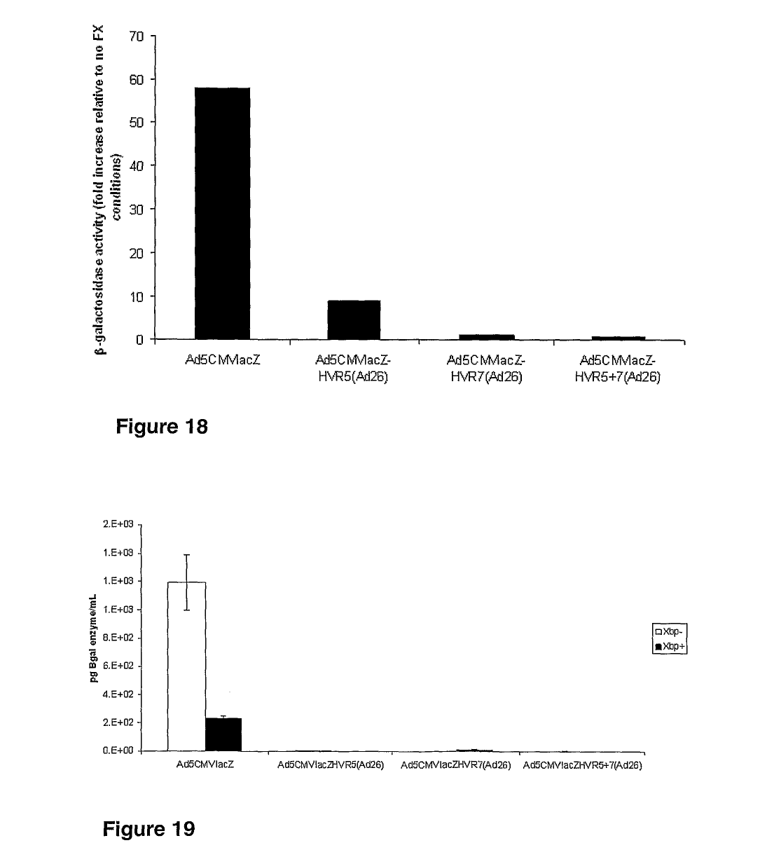

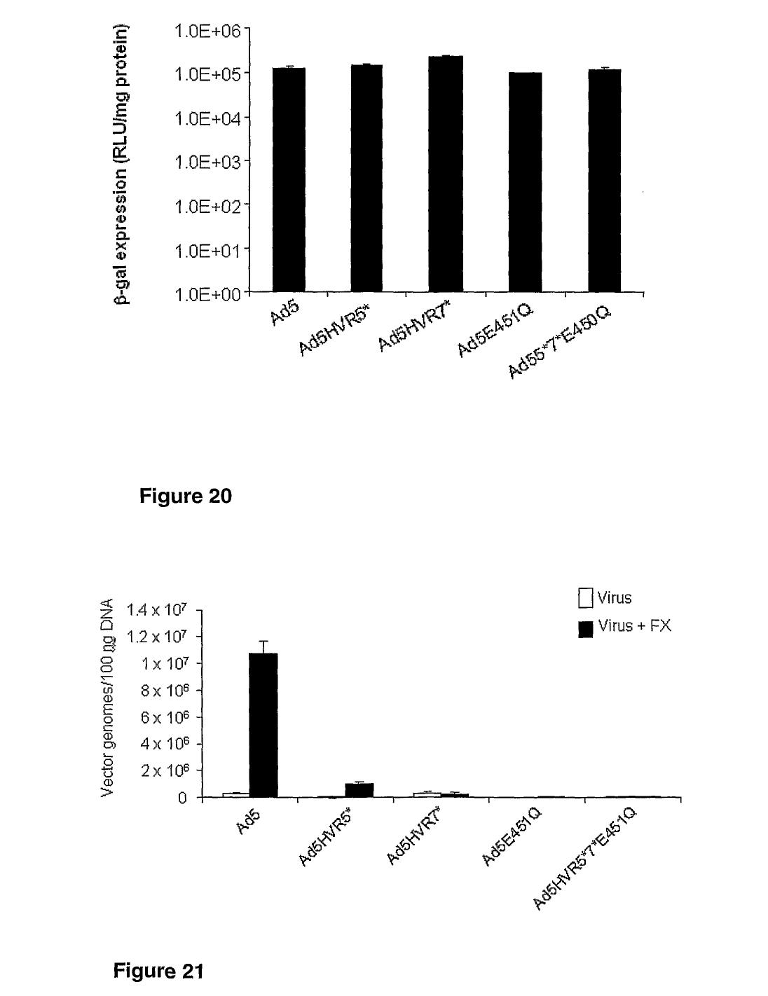

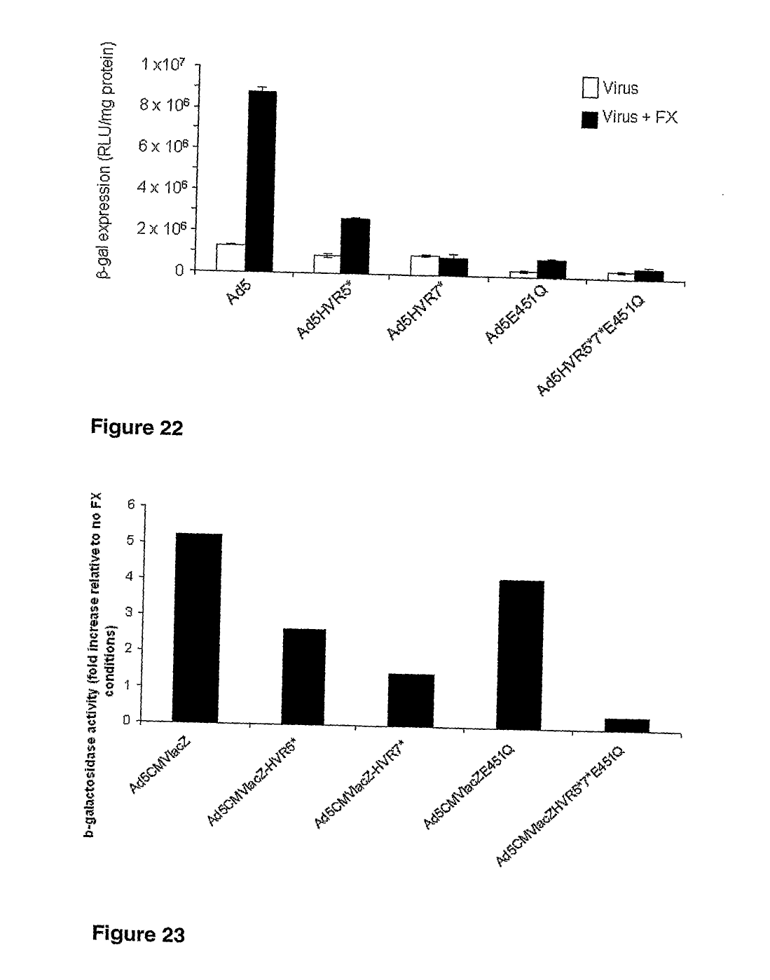

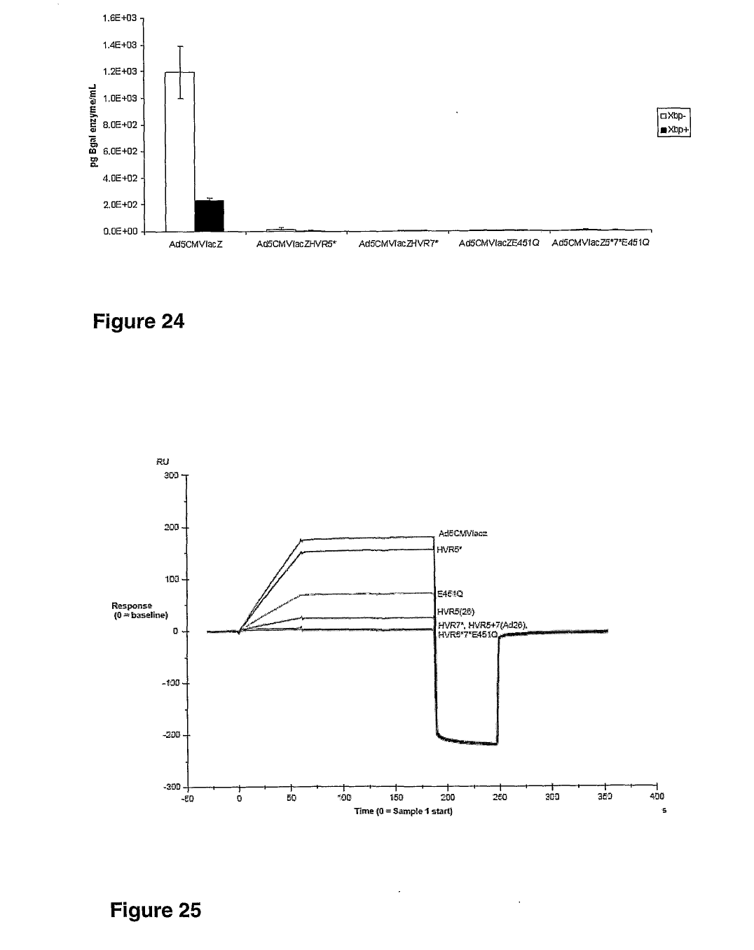

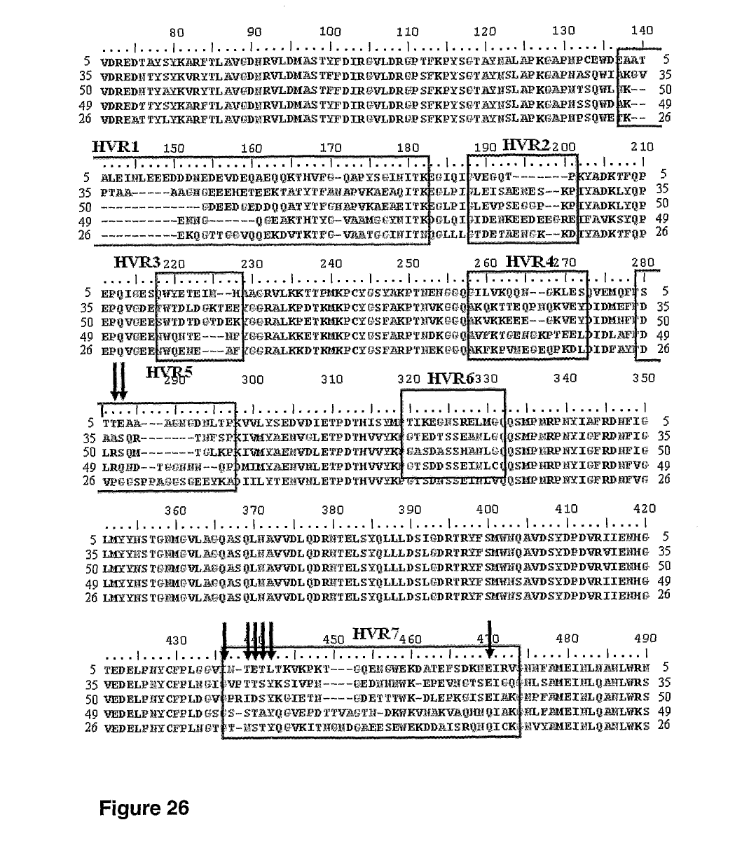

FX is a blood clotting factor containing Gla, EGF1, EGF2 and serine protease (SP) domains. The domain structure of the FX protein is shown in FIG. 1, and representative sequences of FX from different species are shown in FIG. 13.

The Gla domain contains 45 amino acids, including a number of genetically encoded glutamic acid residues all of which are converted to γ-carboxyglutamic acid. The Gla domain has been found to be involved in the interaction with the hexon protein. Isolated Gla domains (or fragments thereof capable of binding hexon protein) find use in a number of aspects of the invention. Such a Gla domain may have at least 800, at least 85%, at least 900 or at least 95% sequence identity to one of the Gla domains illustrated in FIG. 13, such as the human Gla domain. Typically the γ-carboxyglutamic acid residues will be conserved, since they are thought to play a role in calcium binding and the interaction with the hexon protein is calcium dependent. Methods performed in vitro which rely on interaction between Gla and hexon will typically take place in the presence of calcium ions. Constructs comprising Gla domain may also comprise one or more other domains from FX, such as the EGF1 domain, but possibly also the EGF2 domain. Typically they do not comprise a SP domain, but if SP is present, then it may comprise a mutation or modification which reduces or ablates binding to heparan sulphate proteoglycans. An EGF1 domain, EGF2 domain or SP domain may have at least 800, at least 85%, at least 90% or at least 95% sequence identity to one of the corresponding domains illustrated in FIG. 13.

### Factor X-Binding Protein

Factor X-binding protein (X-bp) is a protein isolated from the venom of Deinagkistrodon acutus. It binds the FX Gla domain and is capable of inhibiting the interaction with hexon protein. It is composed of two subunits. Sequences of the subunits and the structure of a complex with FX are shown in Mizuno et al., 2001.

Thus Chain A is believed to have the sequence:

DCSSGWSSYE GHCYKVFKQS KTWADAESFC TKQVNGGHLV SIESSGEADF VGQLIAQKIK
SAKIHVWIGL RAQNKEKQCS IEWSDGSSIS YENWIEEESK KCLGVHIETG FHKWENFYCE
QQDPFVCEA

Chain B is believed to have the sequence:

DCPSDWSSYE GHCYKPFNEP KNWADAENFC TQQHTGSHLV SFQSTEEADF VVKLAFQTFD
YGIFWMGLSK IWNQCNWQWS NAAMLKYTDW AEESYCVYFK STNNKWRSIT CRMIANFVCE
FQA

The invention extends to the use of analogues of X-bp which contain one or more modifications or mutations relative to the sequences given above but retain the ability to bind FX Gla. For example, such an analogue may comprise a first polypeptide chain having at least 80% sequence identity to Chain A and a second polypeptide chain having at least 80% sequence identity to chain B, wherein said analogue is capable of binding FX Gla.

It may be desirable for the analogues to retain one or more of E98 and K100 of Chain A, and/or K100, N103, R107 and R112 of Chain B, since these residues are believed to be involved in contacting the Gla domain.

It may also be desirable to retain one or more of S20, S61, A62, K63, H65, E99, E115, N116, F117, Y118, E120, Q121, Q122, D123 of Chain A, and Y61, I63, Y95, Y98, 5108 of Chain B, since these are believed to be involved interactions with water molecules in the binding of Gla. Conservative substitutions may be acceptable at these sites.

### Sequence Homology, Mutations and Modifications

Reference in this specification to mutations or modifications of particular amino acid sequences may include point mutations, i.e. deletions, substitutions and insertions of particular amino acids. Non-conservative substitutions may be particularly suitable for generating hexon variants having altered binding to FX, as a mutant or variant having a non-conservative mutation is less likely to retain original function than one having a conservative substitution. Conservative substitutions may also be useful in the generation of variant hexon proteins, however, for example in the scaffold regions. Conservative mutations may also be found in constructs containing FX (e.g. Gla) sequences or X-bp sequences, where the intention is to retain a wild-type function, such as binding to hexon or to Gla.

However it will be clear that modification need not always involve a point mutation. For example, it may involve exchanging a stretch of amino acid sequence for a replacement stretch of amino acid sequence, of the same or different length, and with more or less sequence identity to the original sequence. For example, it may be desirable to replace a HVR sequence with a randomly generated sequence in order to test for altered Gla binding. Indeed, a population (e.g. “library”) of test proteins, each comprising a different sequence in a given region such as an HVR, may be generated for screening, in order to identify a suitable sequence having desired binding properties. The skilled person will be well aware of suitable techniques for construction and screening of such libraries.

A conservative substitution may be defined as a substitution within an amino acid class and/or a substitution that scores positive in the BLOSUM62 matrix as shown below, thus a non-conservative substitution maybe defined as a substitution between amino acid classes, or which does not score positive in the BLOSUM62 matrix.

According to one classification, the amino acid classes are acidic, basic, uncharged polar and nonpolar, wherein acidic amino acids are Asp and Glu; basic amino acids are Arg, Lys and His; uncharged polar amino acids are Asn, Gln, Ser, Thr and Tyr; and non-polar amino acids are Ala, Gly, Val, Leu, Ile, Pro, Phe, Met, Trp and Cys.

According to another classification, the amino acid classes are small hydrophilic, acid/acidamide/hydrophilic, basic, small hydrophobic and aromatic, wherein small hydrophilic amino acids are Ser, Thr, Pro, Ala and Gly; acid/acidamide/hydrophilic amino acids are Asn, Asp, Glu and Gln; basic amino acids are His, Arg and Lys; small hydrophobic amino acids are Met, Ile, Leu and Val; and aromatic amino acids are Phe, Tyr and Trp.

Conservative substitutions, which score positive in the BLOSUM62 matrix, are as follows:

| | Original Residue | | | | | | | | | | | |
|---|---|---|---|---|---|---|---|---|---|---|---|---|
| | C | S | T | P | A | G | N | D | E | Q | H | R |
| Substitution | — | T A N | S | — | S | — | S D H | N E | D Q K | E R K | N Y | Q K |

| | Original Residue | | | | | | | |
|---|---|---|---|---|---|---|---|---|
| | K | M | I | L | V | F | Y | W |
| Substitution | E Q R | I L V | M L V | M I V | M I L | Y W | H F W | F Y |

Percent (%) amino acid sequence identity with respect to a reference sequence is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. % identity values may be determined by WU-BLAST-2 (Altschul et al., Methods in Enzymology, 266:460-480 (1996)). WU-BLAST-2 uses several search parameters, most of which are set to the default values. The adjustable parameters are set with the following values: overlap span=1, overlap fraction=0.125, word threshold (T)=11. A % amino acid sequence identity value is determined by the number of matching identical residues as determined by WU-BLAST-2, divided by the total number of residues of the reference sequence (gaps introduced by WU-BLAST-2 into the reference sequence to maximize the alignment score being ignored), multiplied by 100.

Percent (%) amino acid similarity is defined in the same way as identity, with the exception that residues scoring a positive value in the BLOSUM62 matrix are counted. Thus, residues which are non-identical but which have similar properties (e.g. as a result of conservative substitutions) are also counted.

References in this specification to an amino acid of a first sequence “corresponding to” an amino acid of a second sequence should be construed accordingly. That is to say, residues which align with one another when the two sequences are aligned as described above, can be considered to correspond to one another.

### Antibodies and Other Binding Agents

Various aspects of the invention may make use of antibodies. An antibody may have the complete native antibody structure, consisting of two complete heavy chains linked by disulphide bonds to two complete light chains, appropriately folded to form two Fab regions and a Fc region, optionally with glycosylation. However it is well known that fragments of a whole antibody can perform the function of binding antigens. The term “antibody” is therefore used herein to encompass any molecule comprising the antigen binding fragment of an antibody. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding member (Bird et al, Science, 242, 423-426, 1988; Huston et al, PNAS USA, 85, 5879-5883, 1988).

The skilled person will be well aware of other binding agents which may be used to bind to a specific target. These agents typically form part of a “specific binding pair”. This term is used to describe a pair of molecules comprising a binding agent and a binding partner which have particular specificity for each other and which in normal conditions bind to each other in preference to binding to other molecules. Examples of specific binding pairs are antibodies and their cognate epitopes/antigens, ligands (such as hormones, etc.) and receptors, avidin/streptavidin and biotin, and lectins and carbohydrates.

Molecular imprints may also be used as binding agents. These may be made by forming a plastic polymer around a target analyte, extracting the analyte from the formed polymer, and then grinding the polymer to a fine powder, as described in Nonbiological Alternatives to Antibodies in Immunoassays; Principles and Practice of Immunoassay (second edition) Chapter 7 pp 139-153 Ed C P Price & D J Newman (1997).

Aptamers may also be used as binding agents. These are DNA or RNA molecules, selected from libraries on the basis of their ability to bind other molecules. Aptamers have been selected which can bind to other nucleic acids, proteins, small organic compounds, and even entire organisms.

### Pharmaceutical Compositions

These compositions may comprise, in addition to the active agent, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes or topical application. Intravenous, intramuscular or subcutaneous administration is likely to be appropriate in many instances, but other methods of administration are possible.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection (which may or may not be at the site where immune stimulation is desired), the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Administration is preferably in an “effective amount” sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Suitable carriers, adjuvants, excipients, etc. can be found in standard pharmaceutical texts, for example Remington's Pharmaceutical Sciences, 20th Edition, 2000, pub. Lippincott, Williams & Wilkins; and Handbook of Pharmaceutical Excipients, 2nd edition, 1994. The skilled person will be capable of ascertaining a suitable dose for any given situation.

The compositions and methods described herein are preferably used for administration to and treatment of mammals, more preferably primates (e.g. humans, apes or monkeys), domestic animals (e.g. feline, canine, etc.), laboratory animals (e.g. rodents including mice and rats, lagomorphs etc.) or livestock animals (e.g. bovine, equine, porcine, etc.).

### EXAMPLES

### The FX Gla Domain is Required for Ad5 Binding

FX is a zymogen of a vitamin K-dependent serine protease with a Gla (γ-carboxylated glutamic acid)-EGF1 (epidermal growth factor-like)-EGF2-SP (serine protease) domain structure (FIG. 1A) that circulates in plasma at a concentration of 8 μg/ml. FX is converted to its active serine protease by a single proteolytic cleavage generating a two chain disulphide linked molecule consisting of a light chain (LC; Gla-EGF1-EGF2) and a heavy chain (HC; SP). There are three calcium ion binding sites in the FX molecule—the Gla domain co-ordinates 7 calcium ions, EGF1 and the SP domain each bind a single calcium ion. The FX-Ad5 interaction is calcium-dependent (Parker et al., 2006). We sought to identify the domain responsible for Ad5 binding. To ascertain whether the N-terminal Gla-EGF1 component of FX bound Ad5 we assessed binding to full length activated human FX (FXa; Gla-EGF1-EGF2-SP) and a human FXa mutant containing the C-terminal EGF2-SP domain (EGF2-SP FXa). The EGF2-SP fragment failed to bind Ad5, which however did show efficient binding to full length FXa (FIG. 1B). This suggests an absolute requirement for Gla-EGF1 in Ad5 binding. This was confirmed using a monoclonal antibody (HX-1) directed to the LC of FX (Gla-EGF1-EGF2) which blocked the interaction of Ad5 with human FX (FIG. 1C) and abolished human FX-mediated gene transfer in vitro [for in vitro studies we used the CAR binding-ablated virus, AdKO1 (Parker et al., 2006) since the use of this virus mimics the CAR-independent liver gene transfer by Ad5 observed in vivo (Parker et al., 2006; Waddington et al., 2007)] (FIG. 1D). To demonstrate the importance of the Gla domain in Ad5 binding we analysed the interaction of Ad5 with Gla domainless human FX (FX-GD) by SPR. The effect of the FX Gla domain opposes our earlier report (Parker et al., 2006) where we showed that both FX and FX-GD evoked equivalent enhancements of in vitro cell transduction mediated by Ad5. The FX-GD utilised was subsequently shown to contain a significant quantity of full length FX by western blot analysis. For the present study we therefore used FX-GD additionally purified by affinity chromatography using an anti-Gla antibody. Whereas Ad5 bound to full-length human FX in a concentration-dependent manner, Ad5 failed to bind human FX-GD (FIG. 1E). In contrast HX-1 bound to both full-length FX and FX-GD (data not shown). We attempted to produce purified Gla domain alone by enzymatic digestion of FX but insufficient quantities for analysis were obtained. To confirm the importance of this interaction in vivo, we pre-treated mice with warfarin to ablate circulating levels of functional vitamin K-dependent zymogens and showed that human FX-GD failed to rescue Ad5-mediated liver transduction (FIG. 1F) despite achieving ‘normal’ (8 μg/ml) physiological plasma concentrations of FX (98%) and FX-GD (106%) as measured by human FX specific ELISA at 45 minutes post-infection with Ad5 (75 minutes post-injection of zymogen). Hence, the FX Gla domain is required for interaction with Ad5.

### FX Binds to the Ad5 Hexon

To determine the FX-binding site on Ad5, we initially focused on the Ad5 fiber since it determines infectivity via CAR (Bergelson et al., 1997; Tomko et al., 1997). The interaction of Ad5 with FIX was identified through a knob-mediated interaction (Shayakhmetov et al., 2005). To evaluate FX binding to Ad5 fiber, we used a series of Ad5 vectors deleted of fiber knob alone, or fiber knob and sequential fiber shaft deletions [Ad5-21R and Ad5-7.5R; (Li et al., 2006), FIG. 2A] or a fully fiberless Ad5 particle (Ad5-ΔF; Von Seggern et al., 1999). Shaft mutants make use of a fiber shaft trimerisation motif for stable trimer formation in the absence of knob (Li et al., 2006). All mutant adenoviruses bound human FX efficiently as determined by SPR (FIG. 2B). We exposed cells to each adenovirus in the presence or absence of physiological levels of human FX, and quantified cell binding. In each case the level of binding is low in the absence of FX but strongly enhanced in its presence, even for fiberless particles (FIG. 2C). This suggests that FX binds an alternate Ad5 capsid protein. Although the hexon is thought to act principally as a structural capsid protein, based on its abundance and exposure at the virion surface we next used SPR to determine whether FX bound hexon. We observed a direct, calcium-dependent high affinity interaction between purified Ad5 hexon and human FX (FIG. 2D). The calculated ka and kd values of the FX for immobilised hexon were 2.28×10⁶ (1/Ms) and 4.5×10⁻³ (1/s) giving an overall KD of 1.95×10⁻⁹. When FX was immobilised and hexon was injected across the biosensor calculated ka and kd values were 2×10⁵ (1/Ms) and 4.1×10⁻³ (1/s) giving an overall KD of 1.94×10⁻⁸. To assess the affinity of FX for the intact virus Ad5 was biotinylated and coupled to a streptavidin coated biosensor chip. The calculated ka and kd values were 1.2×10⁶ (1/Ms) and 2.23×10⁻³ (1/s) giving an overall KD of 1.83×10⁻⁹ (data not shown).

### Electron Cryomicroscopy and Three-Dimensional Reconstruction of Ad5 Bound to FX

To visualise the interaction between Ad5 and FX, 3D reconstructions were calculated from cryomicrographs of both Ad5 and Ad5 complexed with FX. A total of 251 Ad5 and 305 FX-Ad5 virus particle images were used to compute reconstructions at 26 Å A and 23 Å resolution, respectively. These maps clearly reveal that the principal point of contact between Ad5 and FX occurs within the cup formed at the centre of each hexon-trimer, supporting the FX-hexon interaction seen by SPR. The structural information from the FX component that extends from the capsid surface is noisy and attenuated due to low occupancy (<50%) and incoherent averaging with FX components bound to adjacent hexons. The FX density at the binding site is significantly stronger, and shows symmetry congruent with the hexon trimer. Such symmetry is not present in FX, suggesting that FX molecules bind to one of three potential binding sites in each hexon trimer, occluding the two remaining symmetry related sites. FX density is then subject to three-fold averaging in the reconstruction. Modelling this interaction by fitting a high-resolution model of FX (Venkateswarlu et al., 2002) to our reconstructions strongly support this interpretation. Moreover, SPR analysis indicates a stoichiometery of binding of 205 FX molecules per virus particle consistent with one FX molecule binding to each hexon trimer. Interestingly, the region containing the FX binding site in hexon is characterised by the presence of hypervariable regions (HVRs) (Rux and Burnett, 2000). We investigated this by SPR analysis of an Ad5 mutant in which all HVRs have been replaced by those from Ad48 [Ad5HVR48; (Roberts et al., 2006) (FIG. 3A). The remainder of the capsid structure is entirely derived from Ad5. Ad48 does not bind FX (FIG. 3B) and neither did the Ad5HVR48 mutant (FIG. 3B). Next, we determined the effect of HVR replacement on infectivity. As Ad5HVR48 contains a wild type Ad5 fiber without a CAR binding mutation, we used human cells with low CAR levels (SKOV3 cells) for in vitro dissection of FX-mediated cell binding and transduction mediated by Ad5, Ad5HVR48 and Ad48. The HVR replacement resulted in a loss of FX-mediated enhancement of cell binding (FIG. 3C) and transduction (FIG. 3D). An identical effect was obtained using the MDA-MB-435 cell line (FIG. 7). We next assessed liver gene transfer in mice mediated by Ad5 and Ad5HVR48. Liver transduction, measured at 0.3 doses 48 h post-intravascular injection demonstrated substantial reduction with Ad5HVR48 compared to Ad5 (FIG. 3E). At the highest dose (5×10¹⁰ VP/mouse) an over 600-fold reduction in liver transgene expression was observed. This contrasted with direct intramuscular injection, where Ad5- and Ad5HVR48-driven transgene expression was not different (FIG. 3E). Thus, replacement of the Ad5 hexon HVRs with those from Ad48 ablated FX binding, cell binding and transduction in vitro and reduced liver transduction in vivo.

### Liver Gene Transfer by the FX-Ad5 Complex

Blood coagulation proteases have evolved versatile and sensitive mechanisms for controlling the specificity of protein substrate recognition mediated by surface sites on the SP domain that are physically separate from the active site residues (exosites). The FX SP domain contains a heparin binding exosite (exosite II) (Rezaie, 2000). We had previously shown that FX enhanced transduction of cells required heparan sulphate proteoglycans (Parker et al., 2007; Parker et al., 2006) and therefore investigated a role for this exosite in transduction of the FX-Ad5 complex. We employed naturally occurring anti-coagulants that bind FX exosite II: nematode anticoagulant peptide-2 (NAPc2), an 85 amino acid polypeptide that is a potent FX-dependent inhibitor and binds human FX with high affinity (KD=7.28×10⁻¹¹M; Table 1) (Murakami, 2007), and Ixolaris a 12 kDa two-Kunitz domain protein isolated from tick salivary gland that also binds human FX with high affinity (KD=1.30×10⁻⁹M; Table 1) (Monteiro et al., 2005). SPR analysis revealed high affinity interaction between either NAPc2 or FX and Ixolaris and human FX, however saturating amounts of either had no effect on Ad5 binding to human FX (FIG. 4A). NAPc2 or Ixolaris eliminated the ability of FX to enhance Ad5 transduction in vitro, an effect observed at concentrations at or above the dissociation constant (KD) for NAPc2:FX and Ixolaris:FX binding (Table 1; FIG. 4B). This effect was due to reduced cell surface binding of FX-Ad5 in the presence of inhibitor (FIG. 8). In vivo, pre-incubation of FX with NAPc2 or Ixolaris prior to injection into warfarin-treated mice, led to a substantial reduction in FX liver rescue (FIG. 4C). Hence, binding of human FX to the Ad5 hexon through the Gla domain leads to cell transduction in vivo mediated through a heparin binding exosite in the FX SP domain.

TABLE 1
Kinetic affinities of FX inhibitors for human and mouse
FX determined by SPR

| | Human FX | | | Mouse FX | | |
|---|---|---|---|---|---|---|
| | ka(1/Ms) | kd (1/s) | Kd (M) | ka (1/Ms) | kd (1/s) | Kd (M) |
| X-bp | 1.96 × 10⁶ | 2.30 × 10⁻⁴ | 1.18 × 10⁻¹⁰ | 2.96 × 10⁵ | 3.1 × 10⁻⁴ | 1.04 × 10⁻⁹ |
| Napc2 | 8.6 × 10⁶ | 6.23 × 10⁻⁴ | 7.28 × 10⁻¹¹ | 1.8 × 10⁷ | 0.31 | 1.70 × 10⁻⁸ |
| Ixolaris | 1.35 × 10⁵ | 1.75 × 10⁻⁴ | 1.30 × 10⁻⁹ | 1.57 × 10⁶ | 8.1 × 10⁻⁷ | 5.15 × 10⁻¹³ |

### Pharmacological Blockade of Ad5 Hexon Binding to FX Blocks, Gene Transfer In Vivo

The treatment of mice with warfarin offers the potential to study each vitamin K-dependent coagulation factor (FVII, FIX, FX, Protein C) in isolation by complementation (Parker et al., 2006). Under such conditions, FX is the only coagulation factor capable of rescuing liver transduction mediated by Ad5 (FIG. 9). The affinity of binding of each coagulation factor to purified Ad5 hexon was quantified by SPR (FIG. 10), again showing differential kinetics for each coagulation factor and the predominant role for FX in this interaction. It is important to ascertain the global importance of the FX-Ad5 interaction in the presence of all other vitamin K-dependent zymogens under physiological conditions in vivo. This is essential for further understanding of the importance of the interaction and how this may be used to design and implement inhibitors to block liver transduction by Ad5. X-bp is a 29 kDa protein, isolated from Deinagkistrodon acutus (the hundred pace snake), that binds with high affinity to the Gla domain of human and murine FX (Atoda, 1998) (Table 1). We thus utilised X-bp to define the effect of blockade of the FX-Gla domain:Ad5 interaction on cell transduction in vitro and in vivo. X-bp blocked the human FX-Ad5 interaction as evidenced by SPR further demonstrating the importance of the Gla domain in FX binding to Ad5 (FIG. 5A). In vitro, pre-incubation of human FX with X-bp at equimolar or higher concentrations prior to addition of Ad5 to cells abolished the FX-mediated enhancement in Ad5 transduction (FIG. 5B). In vivo, X-bp when pre-incubated with FX prior to injection blocked the ability of human FX to rescue liver transduction by Ad5 in warfarinised mice (FIG. 5C). Importantly, pre-injection of wild type control (non-warfarinised) mice with X-bp substantially reduced liver transduction mediated by a subsequent injection of Ad5 (FIG. 5D). These findings were repeated in a second species (rat; FIG. 11). Hence, based on our knowledge of the Ad5 hexon:FX interaction we have identified an efficient and simple pharmacological approach to block Ad5-mediated liver transduction following intravascular delivery.

### FX Binding to Other Human Ad Serotypes

The human adenovirus family is substantial with over 50 serotypes, divided into subgroups A-F on the basis of hemagglutination, oncogenic properties and DNA sequence identity. Many adenoviruses are being exploited for application to treat human diseases or for vaccination. A number of these approaches use adenoviruses derived entirely from alternate serotypes (Abbink et al., 2007; Lecollinet, 2006; Sakurai, 2006; Vogels et al., 2003) although many strategies simply use the Ad5 capsid but exchange the Ad5 fiber for the fiber from an alternate serotype (also called pseudotyping) (Gaggar et al., 2003; Shayakhmetov et al., 2002; Shayakhmetov et al., 2000). Clearly, since FX binds the Ad5 hexon, the pseudotyping strategy will result in viruses that still possess FX-binding capacity and may possess FX-mediated infectivity effects in vitro and in vivo (Parker et al., 2007; Waddington et al., 2007). There are distinct advantages to using complete serotypes that have rare pre-existing immunity in the human population (Abbink et al., 2007; Vogels et al., 2003) for clinical application. The hexon protein is highly conserved between different serotypes (70-80% sequence identity) but in the HVRs there are distinct amino acid sequence profiles. Phylogenetic analysis of the amino acid sequences of the HVRs identified two distinct clades corresponding to the D subgroup and the A, B and C subgroups (FIG. 6A) (Madisch et al., 2005). The observed difference in amino acid sequences in the HVRs most probably reflects the early divergence of subgroup D from A, B and C. We tested the ability of diverse human Ad serotypes to bind FX by SPR (FIGS. 6A,B). Three phenotypes were detected—adenoviruses that bind strongly, indicated by ++ or +++ (e.g. Ad5, Ad2, Ad50, Ad16), adenoviruses that show weak FX binding indicated by + (e.g. Ad35, Ad3) and adenoviruses that do not bind FX at all indicated in blue (e.g. Ad48, Ad26)(FIG. 6A, B). Viruses that do not bind FX all belong to the D subgroup (FIG. 6A). We next assessed whether binding to FX correlated with FX-sensitivity in cell binding and transduction in vitro and in vivo, focusing on Ad35 (weak binding) and Ad26 (no binding), vectors that are in clinical development, and comparing this to Ad5 (strong binding). Kinetic analysis by SPR for FX binding to Ad35 and Ad26 revealed for Ad35a ka of 1.1×10⁵ (1/Ms), a kd of 5.7×10⁻³ (1/s) giving an overall KD of 5.2×10⁻⁵ and for Ad26 no detectable binding even at 50 μg/ml FX (data not shown). The KD for Ad35 is, as expected, significantly weaker than the Ad5:FX interaction. In vitro, Ad binding to, and transduction of HepG2 cells was FX-sensitive for Ad5 but not for Ad35 and Ad26 (FIG. 6C). For Ad35, injection into CD46 transgenic mice either in the presence or absence of X-bp pre-injection showed lung targeting (FIG. 6D). For Ad26, intravascular administration into mice revealed a complete lack of liver transduction, either with or without pre-injection of X-bp (not shown). In vivo analysis of Ad48, a second non-FX binding serotype also showed a lack of hepatic tropism (not shown). Taken together with the observations on Ad26, this indicates that vectors derived from adenoviruses that show weak or no binding to FX are not influenced by the FX pathway in vivo. This has important implications for vector usage and engineering strategies for gene therapy applications.

FIG. 14 represents the cloning strategy used for the generation of hexon mutant adenoviral vectors. All mutations and sequences were inserted by site directed mutagenesis or PCR amplification from plasmids containing the Ad5 or Ad26 serotypes. All PCR fragments containing the desired mutagenesis were cloned in intermediate plasmids to obtain shuttle plasmids with Ad5-hexon flanking regions. Homologous recombination was performed in BJ5183 bacteria prior to the packaging and amplification of the viral genomes in HEK293 cells. All final and intermediate cloning steps were checked by sequencing.

All viruses grew efficiently in HEK293 cells and titers were comparable to Ad5CMVlacZ control virus with unmodified hexon. To demonstrate that the mutations incorporated in the hexon protein did not decrease the transduction ability of the virus, we assessed transduction in CAR-permissive HeLa cells (FIGS. 15 and 20). As shown, all viruses show capacity to infect HeLa cells although we observed reduced infectivity for viruses containing modified HVR5 regions (FIG. 15).

In order to demonstrate the ability of these viruses to bind cellular receptors, we performed binding experiments in SKOV3 cells (low CAR expression), in the presence or absence of factor X. FIGS. 16 and 21 represent the viral genome copies bound with cellular receptors after incubating the virus at 4° C. for 1 hour on SKOV3 cells. All HVR swap Ad vectors mediated lower binding than Ad5, however HVR7 completely abolished binding in the presence or absence of FX (FIG. 16). Similarly, HVR7* diminished FX binding in comparison to other point mutants and control Ad5CMVlacZ (FIG. 21).

Quantification of β-gal expression was performed to analyse the transduction efficiency for each virus in SKOV3 cells in the presence or absence of factor X. HVR5 mutant vector demonstrated 6 times lower transduction efficiency (FIGS. 17 and 18). However, HVR7 swap containing viruses showed no cell transduction. As for cell binding, HVR7* point mutant vector has the most influence on cell transduction compared to all other point mutant adenoviral vectors (FIGS. 22 and 23).

Following intravascular delivery, adenovirus serotype 5 displays clear tropism for liver. In order to analyze the transduction efficiency of hexon mutant adenovirus in vivo, we performed an ELISA assay for measuring β-galactosidase activity in 200 ug of liver extracts (FIGS. 19 and 24) 48 hours following intravascular delivery of each adenoviral vector. All mutant hexon adenovirus showed a total abolition of liver transduction in the presence or absence of Xbp in contrast to Ad5CMVlacZ. FIG. 25 represents the capacity of viral particles to bind FX using SPR. Although HVR5 and HVR7 exchange reduces FX binding, replacement of HVR7 essentially abolished FX binding by SPR (FIG. 25). Similarly, HVR7* seems to play a major role in FX binding in comparison to the mutants HVR5* or E450Q [E451Q in the sequence having accession nos. AAW65514.1; GI:56177707].

FIG. 26 represents part of the nucleotide sequence of adenovirus hexon from different serotypes. Arrows indicate the critical or putative contact residues for FX binding in Ad5 hexon HVRs. HVR5 and HVR7 regions seem to provide the major contribution for FX interaction.

### Discussion

We reveal an unexpected role for the Ad5 hexon protein for defining virus infectivity in vivo. The complex of Ad5 with FX, mediated through a molecular interaction between the FX Gla domain and the hexon HVRs, is the major pathway leading to in vivo delivery of adenovirus to hepatocytes from the bloodstream. This is supported by the over 150-fold reduction in liver transduction mediated by Ad5 in mice pre-injected with X-bp, a snake venom-derived protein that blocks the molecular interaction of FX Gla and Ad5 hexon. Moreover, the chimeric Ad5 vector Ad5HVR48, which has only the HVRs of Ad48 swapped into the Ad5 hexon, displays abolished FX binding activity by SPR and in vitro assays as well as a substantial reduction in liver gene transfer. It is important to remark that all other capsid components in Ad5HVR48 are Ad5. The Ad5 fiber has no function in FX binding as swapping Ad5 hexon HVRs for those from the non-binding Ad48 abolishes FX binding capacity. This pathway has fundamental implications for adenovirus biology and vector design for human gene therapy. Moreover, the role of the Ad hexon protein in cellular infectivity provides important information that may lead to the more effective development of safer gene therapies. Previously, we and others have reported that liver transduction by CAR binding and CAR binding-mutated Ad5 vectors are equal (reviewed in Nicklin et al., 2005; Waddington et al., 2007). Clearly, fiber modifications have been shown to impact liver transduction, including Ad5 vectors pseudotyped with alternative fibers and Ad5 vectors devoid of the putative heparan sulfate proteoglycan binding site in the fiber shaft (Koizumi et al., 2006; Smith et al., 2003; reviewed in Nicklin et al., 2005). However, mutation at this site may confer fiber rigidity since vectors possessing this mutation maintain the ability to bind cells but are not able to mediate transduction (Bayo-Puxan et al., 2007; Kritz et al., 2007). The lack of influence on liver transduction following mutation of the fiber and/or penton proteins (Nicol et al., 2004, Smith et al., 2003, Martin et al., 2003) suggests that these mutations have not identified the natural mechanism for Ad5 localisation to hepatocytes following intravascular delivery. This is supported by the findings in our study as either pharmacological intervention or mutation of the Ad5 hexon protein block Ad5:FX interaction and liver transduction by Ad5 in vivo. Importantly, Ad5HVR48 is still able to mediate transduction at a comparable level to Ad5 following direct intramuscular injection thus showing that this virus still retains the ability to interact with its other receptors in the appropriate in vivo setting. Together these findings support the assertion that interaction between the Ad5 hexon and FX is the major pathway that the virus utilises to achieve localization to hepatocytes via the bloodstream. The roles of other capsid proteins, including the fiber and penton in the entire liver transduction process relating to the FX-mediated pathway will be important to ascertain. Transduction is a complex series of mechanisms including cell attachment, internalization and nuclear trafficking and our modeling suggested projection of FX from the virion surface thus potentially sterically influencing fiber-mediated interactions. Importantly, liver transduction is often a serious concern following local application of adenovirus in vivo (Hiltunen et al., 2000), hence simultaneous administration of an FX-Ad5 blocking agent may prevent such occurrence, thus improving safety.

Blocking the hexon:FX interaction could be achieved by HVR exchange as evidenced for Ad48 or by selective mutagenesis, although careful analysis of full serotype tropism would be required, ideally focusing on serotypes that offer lower sero-prevalence with respect to pre-existing antibodies in the human population (Roberts et al., 2006). This would create adenoviruses with both lower sero-prevalence in human populations and lacking FX binding. Alternatively, mutagenesis strategies for Ad5 hexon, focusing on key interacting locales in HVRs to ablate hexon:Ad5 interactions will result in Ad5 mutants devoid of FX binding.

This study has implications for vector design using pseudotyping strategies involving swapping of alternate (non-Ad5) fibers onto the Ad5 capsid. Fibers derived from sub-group B Ads that bind CD46 are of particular interest, as they mediate enhanced Ad uptake into CD46 positive tissues, including tumours (Gaggar et al., 2003; Sakurai, 2006; Tuve, 2006). Since we show that Ad35 only weakly interacts with FX, it can be envisaged that development of full serotype Ad35 vectors maybe more preferential than using a fiber-pseudotyping strategy alone since the Ad5 hexon will still bind FX within the context of a pseudotyped virus. This is exemplified by our recent analysis of adenoviruses pseudotyped with fibers from subgroup D, all of which showed direct binding to FX by SPR (in comparison to the divergent data on the full subgroup D viruses presented here), resulting in enhanced FX mediated cell surface binding and transduction (Parker et al., 2007). Furthermore, in vivo targeting of Ad5/47 (a virus pseudotyped with the fiber from the subgroup D virus Ad47) to the liver was sensitive to warfarin treatment suggesting the binding of FX to Ad5 hexon leads to efficient liver targeting and is dominant over any fiber effects (Waddington et al., 2007).

Taken together, this study identifies an unexpected function of adenovirus hexon in mediating in vivo liver gene transfer by Ad5 through recruitment of host FX to the hexon HVR and cell binding of the resulting complex through the FX serine protease. Moreover, this is an effect applicable to a number of alternate human serotypes and has implications for adenovirus vector development and safety for application to human gene therapy.

### Experimental Procedures

### Materials

Purified blood coagulation factors (FX, FX-GD, protein C and FXI) were purchased from Haematologic Technologies (Essex Junction, Vt.). Recombinant (r) FIX (BeneFIX) was from Wyeth (Philadelphia, US). rFVII was sourced as described (Parker at al., 2006). rFX EGF2-SP was a kind gift of Daniel Johnson (Cambridge, UK). FX-GD was prepared by chymotrypsin digest of full-length FX (purchased from Haematologic Technologies). The FX-GD was further purified by removal of uncleaved full-length FX using affinity chromatography with a monoclonal antibody to γ-carboxy glutamic acid residues (American Diagnostica, Stamford, Conn.). rNAPc2 was a kind gift of Dr G. Vlasuk (Corvas International, San Diego, Calif.). Antibodies were sourced as follows: HX-1 (Sigma, Poole, UK); monoclonal antibody 3570 against γ-carboxyglutamic acid residues (American Diagnostica, Stamford, Conn.). Ad5 hexon was purified from Ad5 infected cells according to the method of Rux et al. (1999). Adenoviruses were propagated in either PER.C6 cells (Vogels et al., 2003) or in 293 cells (Parker et al., 2006). FX levels were determined using a standard sandwich ELISA Asserachrom X:AG kit (Diagnostica Stago Inc., Parsipanny, N.J.). Plasma samples (1:9 ratio of blood: 3.8% sodium citrate anticoagulant) were loaded in duplicate at both 1/50 and 1/200 dilutions. The ELISA recognised both full-length FX and FX-GD with similar efficiency.

### Surface Plasmon Resonance Analysis (SPR)

SPR was performed using a Biacore T100 and a Biacore X (Biacore, Stevenage, UK) (Parker et al., 2006). Coagulation factors were covalently immobilised onto flowcells of CM5 biosensor chips by amine coupling according to the manufacturer's instructions. Virus in 10 mM HEPES pH 7.4; 150 mM NaCl; 5 mM CaCl₂; 0.005% Tween 20 was passed over the chip at 30 μL/min. Sensorchips were regenerated between virus application by injection of 10 mM HEPES pH 7.4; 150 mM NaCl; 3 mM EDTA; 0.005% Tween 20. FXa and EGF2-serine protease FXa were biotinylated with biotin-FPRCK (Haematologic Technologies Inc) at 25° C. overnight and then dialysed. Ad5 was biotinylated using the EZ-link sulfo-NHS-LC-biotinylation kit (Pierce, Rockford, Ill.) according to the manufacturer's instructions. The biotinylated products were coupled to streptavidin coated sensorchips (SA; Biacore) according to the manufacturer's instructions. Rmax was calculated from the immobilized Ad5 (500 RU) using the formula: Rmax=Mw_{FX}/Mw_{Ad5}×R_{I}×S where s=1. To assess binding of Ad serotypes human (h) coagulation FXI and FX were covalently immobilized onto flowcells of a CM5 biosensor chip by amine coupling (hFXI, 8496 RU; hFX, 6460 RU). Ad serotypes (100−0.79×10¹⁰ vp/ml, 8 two-fold serial dilutions were analyzed in duplicate) were passed over the chip at 30 μL/min.

### Kinetic Affinities of FX Inhibitors for Human and Mouse FX Determined by SPR

Human (h) coagulation FXI (731 RU), FX (584 RU), mouse (m) FX (548 RU) and purified Ad5 hexon (521 RU) were covalently immobilized onto flowcells of a CM5 biosensor chip by amine coupling. X-bp, rNapc2 and Ixolaris (10-0.078 μg/ml, 8 two-fold serial dilutions were analyzed in duplicate) or coagulation factors (50-0.78 μg/ml, 7 two-fold serial dilutions analyzed in triplicate) were passed over the chip at 30 μL/min in 10 mM HEPES pH 7.4; 150 mM NaCl; 5 mM CaCl₂; 0.005% Tween 20. Sensorchips were regenerated by injection of glycine pH 2 for the inhibitors and 10 mM HEPES pH 7.4; 150 mM NaCl; 3 mM EDTA; 0.005% Tween 20 for coagulation factors. Kinetic analysis was performed using Biacore T100 evaluation software and fitted using a heterogeneous ligand model.

### In Vitro Experiments

Human HepG2 cells were cultured in Dulbecco's Modified Eagle's Medium (DMEM, Biowhittaker), supplemented with 2 mM L-glutamine (Invitrogen, Paisley, UK) and 10% fetal calf serum (FCSPAA Laboratories, Yeovil, UK). SKOV-3 and MDA-MB-435 cells were maintained in RPMI 1640 media (Invitrogen) with 5% serum. For transgene expression, 2×10⁴ cells/well were plated into 96-well plates and transferred to serum free media containing 8 □g/ml FX (1 IU/ml; Cambridge Biosciences, Cambridge, UK). Virus was added at 1000 VP/cell for 3 hours at 37° C. Cells were maintained at 37° C. until harvesting at 72 h. In experiments with NAPc2 and Ixolaris, FX was preincubated for 30 minutes. X-bp and FX (1 IU/ml) were mixed at varying molar ratios (10-0.001) for 30 minutes. Expression of β-galactosidase was quantified using Tropix Galacto-Light Plus (Applied Biosystems, Foster City, Calif.) using a Wallac VICTOR2 (PerkinElmer Life and Analytical Sciences, Boston, Mass.) and recombinant β-galactosidase as standard. Protein concentrations were quantified by bicinchoninic acid assay (Perbio Science, Cramlington, UK). All data are expressed as relative light units (RLU) per milligram of protein. Cells were imaged using an Olympus BX40 microscope. Cell binding experiments were performed in 24 well plates and in serum free media +/−FX at 4° C. for 1 h using 1000 VP/cell (unless otherwise stated). Viral and total genomic DNA were isolated using a QIAamp DNA mini kit (Qiagen, Crawley, UK), quantified by Nanoprop [ND-1000 spectrophotometer (Labtech International, Ringmer, UK)]. 100 ng of total DNA was subject to quantitative PCR analysis using an ABI prism 7900HT Sequence detection system (Applied Biosystems, Warrington, UK) using the Power SYBR Green PCR Master Mix as described (Parker et al., 2006). Total Ad genomes were calculated using the SDS 2.3 software and using a standard curve of 10²-10⁷ Ad particles.

### Electron Cryomicroscopy.

Ad5 was incubated in the presence of an approximate three-fold excess of FX overnight at 4° C. Complexed and non-complexed virus was prepared for electron cryomicroscopy as previously described (Adrian et al., 1984). Briefly, 5 μl of virus was loaded onto a freshly glow-discharged Quantifoil holey carbon support film (R2/2 Quantifoil Micro Tools GmbH, Jena, Germany), blotted and plunged into a bath of liquid nitrogen cooled ethane slush. Vitrified specimens were imaged at low-temperature in a JEOL 1200 EXII transmission electron microscope equipped with an Oxford instruments CT3500 cryo-transfer stage. Low-dose focal pair images were recorded at 30,000× magnification on Kodak S0163 film.

### Three-Dimensional Image Reconstruction.

20 and 21 focal pairs of micrographs were selected on the basis of optimal ice-thickness for unlabelled and labelled virus respectively. These were digitised on a Nikon Super Coolscan 9000ED CCD scanner at 4000 dpi resolution, corresponding to 2.18 Å/pixel in the specimen. Scanned micrographs were then converted to PIF (Purdue image format) and binned by a factor of three using the BSOFT image-processing package (Heymann, 2001), giving a final raster step size of 6.54 Å/pixel. X3D was used to select and cut out individual particles, these were then processed to correct imaging artefacts, by determining the defocus of each micrograph and inverting successive oscillations of the contrast-transfer function using the CTFMIX program. Subsequently, full CTF correction was applied, merging defocus paired particle images (Conway and Steven, 1999). To calculate an initial reconstruction for unlabelled Ad5, a common-lines approach was taken using a modified version of the MRC icosahedral reconstruction suite of programs (Crowther et al., 1970; Fuller et al., 1996). Following calculation of a preliminary reconstruction, a modified version of the polar Fourier transform method (PFT2) was used to determine and refine origins and orientations for each particle in both complexed and non-complexed data sets (Baker and Cheng, 1996; Bubeck et al., 2005). Resolution assessment for the final reconstructions was performed by dividing each data set into equal halves. These were used to calculate two independent reconstructions. A number of measures of agreement were calculated for the two density maps, including the Fourier shell correlation and spectral signal to noise ratio, using the BSOFT program bresolve. Reconstructions were visualised using UCSF Chimera (Pettersen et al., 2004). The Ad5:FX reconstruction is deposited in the EM database with accession code EMD-1464.

### In Vivo Methods.

MF-1 outbred mice (Harlan, UK) or CD46 transgenic mice (Olstone et al., 1994), 25-30 g were used for all experiments. In some experiments, 5 mg/kg warfarin suspended in peanut oil was injected subcutaneously 3 and 1 days prior to Ad5 administration to ablate circulating levels of functional vitamin K-dependent coagulation factors as described (Parker et al., 2006; Waddington et al., 2007). For NAPc2 and Ixolaris experiments, 400 μg/kg of NAPc2 or 0.78 mg/kg Ixolaris were pre-mixed with FX 30 minutes prior to intravenous injection of both. For X-bp studies we injected 4.8 mg/kg X-bp intravascularly in warfarin-treated mice or into normal (non-warfarinised) mice 30 mins before injection of adenovirus. For Ad5 vs Ad5HVR48 intravascular injection experiments, mice received 200 μl intravenous clodronate liposomes (Van Rooijen and Sanders, 1994) 24 hours before adenovirus. For intramuscular injections 1×10¹⁰ VP/mouse was injected into the tibialis anterior. Mice were subject to whole-body bioluminescence quantitation (IVIS-50, Xenogen, USA) or were sacrificed for analysis of tissue expression of β-galactosidase as described (Waddington et al., 2007).

### Statistical Analysis

In vitro experiments were performed in triplicate on at least three independent occasions. In vivo experiments were performed with at least four animals per group. Where necessary, data was normalised by logarithmic transformation. Analysis was either by unpaired Student's t test or for multiple comparisons analysis of variance and Tukey's pairwise comparison using MINITAB software.

### Cell Lines

Human embryonic kidney HEK293 cells (American Type Culture Collection, Manassas, Va., USA) were cultured in Dulbecco modified Eagle medium (DMEM; BioWhittaker, Wokingham, United Kingdom) supplemented with 2 mM L-glutamine (Invitrogen, Paisley, United Kingdom), 10% fetal calf serum (FCS; PAA Laboratories, Yeovil, United Kingdom) and 1 mM sodium pyruvate (Sigma, Hertfordshire, United Kingdom). Human ovarian carcinoma SKOV3 cells (N.I.H.) were cultured in RPMI 1640 media (Invitrogen, Paisley, United Kingdom) supplemented with 2 mM L-glutamine, 10% fetal calf serum and 1 mM sodium pyruvate. Human cervical carcinoma HeLa cells were cultured in Dulbecco modified Eagle medium supplemented with 2 mM L-glutamine, 10% fetal calf serum and 1 mM sodium pyruvate. Cell lines were maintained at 37° C. and 5% CO₂.

### Vector Construction

HVR swap modifications were generated by amplifying HVR5 and 7 regions from plasmids containing the entire viral genomes of human serotypes 5 and 26. All hexon point mutant adenoviruses were generated using PCR site-directed mutagenesis. PCR fragments incorporating the desired mutations were cloned into pSC-B using the Strataclone Blunt PCR cloning kit according to the manufacturer's instructions (Stratagene, Leicester, United Kingdom). Modified hexon fragments were then excised by digesting pSC-BHex with BamHI and NdeI and subcloning fragments into pBR.dbb.PmeI-SandI plasmid. Primers 2 KbDIR: (5′ACAGTGGAAAGGTCGACGC3′) and 2 KbREV: (5′ACTTGACTTTCTAGCTTTCC3′) were used to amplify a 1.95 Kb fragment of the left hand flanking region of the adenoviral Ad5 hexon. The 1.95 Kb fragment was excised using NdeI and cloned into pBR plasmids containing modified hexon fragments in order to generate shuttle plasmids for homologous recombination. Adenoviral vectors used in this study were based on the AdEasy, E1/E3 deleted Ad5 Adenoviral vector system (Stratagene, Leicester, United Kingdom). PacI-digested pAdeasy-1 plasmid and PmeI-digested pShuttle-CMV-lacZ were used to generate pAd5CMVlacZ by homologous recombination in BJ5183 bacteria cells. All pBR-based plasmids containing hexon mutants and the 1.95 Kb fragment were digested by EcoRI and recombined with AsiSI-digested pAd5CMVlacZ in BJ5183 bacteria cells. All cloning steps were checked by sequencing using a 3730 DNA analyzer (Applied Biosystems, Warrington, United Kingdom).

Vectors were constructed in which HVR5 or HVR7 of Ad5 hexon protein was replaced by the corresponding HVR from Ad26. These mutations were designated Ad5HVR5(Ad26) and Ad5HVR7(Ad26). The combination of these mutations was designated Ad5HVR5+7(Ad26).

The two point mutations T269P and E270G [shown as T270P and E271G in the sequence having accession nos. AAW65514.1; GI:58177707] were introduced into the Ad HVR5 region. This set of substitutions was designated HVR5*.

The four point mutations I1420G, T422N. E423S and L425Y [shown as I421G, T423N. E424S and L426Y in the sequence having accession nos. AAW65514.1; GI:58177707] were introduced into the Ad HVR7 region. This set of substitutions was designated HVR7*.

Adenoviruses were generated having these two sets of substitutions individually and in combination, both with each other and with other mutations.

The point mutation E450Q [shown as E451Q in the sequence having accession nos. AAW65514.1; GI:58177707] was also introduced into Ad5 hexon. Adenoviruses having this mutation alone in combination with other mutations.

Thus, for example, the mutant designated Ad5HVR5*7*E450Q (or Ad5HVR5*7*E451Q) carries the two substitutions designated HVR5*, the four substitutions designated HVR7* and the single substitution E450Q [E451Q in the sequence having accession nos. AAW65514.1; GI:58177707].

### Vector Amplification

PacI-digested adenoviral genomes were transfected into HEK293 cells in a 6-well plate using lipofectamine 2000 (Invitrogen, Renfrew, United Kingdom) according to manufacturer's instructions. Cells and media were recovered when viral foci were observed 7-10 days post-transfection. Viral particles were amplified through different passages until 20×T150 flasks were reached. Pelleted cells were lysed using Arklone-P (1,1,2-Tricholorotrifluorethane, Riedel-de Haen, United Kingdom) then purified by one-step CsCl gradient centrifugation. Purified adenoviruses were then dialysed into 10 mM Tris-HCl, 1 mM EDTA and 10% glycerol using slide-A-lyzer dialysis cassettes (Thermo Scientific, Winsford, United Kingdom) before being stored at 80° C. Adenoviral particle titers were calculated by determining protein concentrations using the micro BCA Protein Assay Kit (Thermo Scientific, Winsford, United Kingdom) followed by titre calculations using the formula 1 μg protein=4×10⁹ vp.

### FX Coagulation Factor Transduction Experiment

Transduction experiments were performed in a 96 well-format with 5×10⁴ SKOV3 or HeLa cells per well. Cells were infected with Ad5 or hexon mutant adenoviruses at a dose of 1000 vp/cell in serum-free medium in the presence or absence of FX (Haematological Technologies, Vt., USA). FX was used at a final concentration of 10 μg/mL. Infected cells were incubated for 3 hours at 37° C., washed with PBS, and maintained until harvesting 48 hours post-transfection.

### Analysis of β-Galactosidase Transgene Expression In Vitro

β-Galactosidase activity was quantified using Tropix Galacton Plus and Tropix accelerator II (Applied Biosystems, Warrington, United Kingdom) according to manufacturer's instructions. β-Galactosidase activity was quantified using a Wallac VICTOR2 luminometer (Perkin Elmer Life and Analytical Sciences, Boston, Mass., USA). Protein concentrations were calculated using a BCA assay (Thermo Scientific, Winsford, United Kingdom). Values are expressed as relative light units (RLU) per milligram of protein.

### Analysis of Adenovirus Cell Binding Capacity

Adenovirus binding experiments were performed in a 24 well-format with 2×10⁵ SKOV3 cells per well. Cells were pre-chilled at 4° C. for 30 minutes and washed twice with PBS (Invitrogen, Paisley, United Kingdom) prior to adding 1000 vp/cell of Ad5CMVlacZ or hexon modified adenoviruses in the presence or absence of FX in serum free media. FX (Haematological Technologies, Vt., USA) was used at a final concentration of 10 μg/mL. Infected cells were incubated with adenovirus for 1 hour at 4° C., washed twice with PBS then snap-frozen in 200 μl of PBS. DNA was extracted from cell pellets using the QIAmp DNA mini kit (QIAGEN, West Sussex, UK) according to manufacturer's instructions. Viral genomes in 100 ng DNA were quantified by QPCR analysis (Applied Biosystems, 7900HT Sequence Detection System, Warrington, United Kingdom) using Power SYBR Green PCR Master Mix (Applied Biosystems, Warrington, United Kingdom) and 2 μM hexon oligonucleotides (hexon Ad R:5′CGCGGTGCGGCTGGTG3′ and hexon Ad F:5′TGGCGCATCCCATTCTCC3′).

### Analysis of β-Galactosidase Transgene Expression In Vivo

Male MF-1 mice aged between 7-9 weeks (Harlan, Oxon, UK) were used for in vivo experiments. 200 μl Clodronate liposomes (http://clodronateliposomes.org) were injected into the tail-vein 24 hours prior to adenovirus administration. 4.8 mg/Kg X-bp (Waddington, McVey et al. 2008) or PBS vehicle was adminstered by tail-vein injection 30 minutes prior to the intravenous administration of 10¹⁰ viral particles in 100 μl PBS. All animals were sacrificed and dissected 48 hours later. β-Galactosidase transgene expression was analyzed using a β-Gal ELISA kit (Roche, West Sussex, United Kingdom) according to manufacturer's instructions. Liver lobes that had been fixed in 2% para-formaldehyde for 16 hours at 4° C. were staining for p-Galactosidase activity in Xgal staining solution (0.1M phosphate buffer (pH 7.3), 2 mM MgCl₂, 5 mM K₃F₃(CN)₆, 5 mM K₄Fe(CN₆)₆ and 1 mg/ml Xgal (5-bromo-4-chloro-3-indolyl-β-D-galactoside)) at 37° C. overnight.

### Modelling of Ad5Hexon-FX Interaction

The crystal structure of Ad5 hexon (PDB ID 1P30-(Rux, Kuser et al. 2003)) was docked to the unlabelled reconstruction of Ad5, at each of the four unique hexons within the asymmetric unit, using Situs (Wriggers, Milligan et al. 1999) To dock modelled coordinates for FX (Venkateswarlu, Perera et al. 2002) a difference map was calculated by subtracting our 26 Å resolution Ad5 reconstruction from the Ad5-FX reconstruction (filtered to 26 Å). At three of the four unique hexon positions within the asymmetric unit of Ad5, there was sufficient density to fit FX (using Situs). Docked coordinates for Hexon and FX were integrated to create single PDB files using UCSF Chimera (Pettersen, Goddard et al. 2004). To determine the points of contact between hexon and FX, each of the three Hexon-FX models was analysed using the CCP4 program contacts (Bailey, LaFleur et al. 1994).

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

### REFERENCES

Abbink, P., Lemckert, A. A. C., Ewald, B. A., Lynch, D. M., Denholtz, M., Smits, S., Holterman, L., Damen, I., Vogels, R., Thorner, A. R., et al. (2007). Comparative seroprevalence and immunogenicity of six rare serotype recombinant adenovirus vaccine vectors from Subgroups B and D. J Virol 81, 4654-4663.
Adrian, M., Dubochet, J., Lepault, J., and McDowall, A. (1984). Cryo-electron microscopy of viruses. Nature 308, 32-36.
Alba, R et al., (2005) Gene Ther 12, S18-S27.
Alemany, R., and Curiel, D. T. (2001). CAR-binding ablation does not change biodistribution and toxicity of adenoviral vectors. Gene Ther 8, 1347-1353.
Athappilly, F., Murali, R., Rux, J., Cai, Z., and Burnett, R. (1995). The refined crystal structure of hexon, the major coat protein of adenovirus Type 2, at 29 A resolution. 242, 430-455.
Atoda, H. I., Midori; Mizuno, Hiroshi and Morita, Takashi. (1998). Coagulation Factor X-binding protein from Deinagkistrodon acutus venom is a gla domain-binding protein. Biochemistry 37, 17361-17370.
Baker, T. S., and Cheng, R. (1996). A model-based approach for determining orientations of biological macromolecules imaged by cryoelectron microscopy. J Struct Biol 116, 120-130.
Bayo-Puxan, N., Cascallo, M., Gros, A., Huch, M., Fillat, C., and Alemany, R. (2006). Role of the putative heparan sulfate glycosaminoglycan-binding site of the adenovirus type 5 fiber shaft on liver detargeting and knob-mediated retargeting. J Gen Virol 87, 2487-2495.
Bergelson, J. M., Cunningham, J. A., Droguett, G., Kurt-Jones, E. A., Krithivas, A., Hong, J. S., Horwitz, M. S., Crowell, R. L., and Finberg, R. W. (1997). Isolation of a common receptor for coxsackie B viruses and adenoviruses 2 and 5. Science 275, 1320-1323.
Bubeck, D., Filman, D., Cheng, N., Steven, A., Hogle, J., and Belnap, D. (2005). The structure of the poliovirus 135S cell entry intermediate at 10-angstrom resolution reveals the location of an externalized polypeptide that binds to membranes. J Virol 79, 7745-7755.
Conway, J., and Steven, A. (1999). Methods for reconstructing density maps of “single” particles from cryoelectron micrographs to subnanometer resolution. J Struct Biol 128, 106-118.
Crowther, R., Amos, L., Finch, J., De Rosier, D., and Klug, A. (1970). Three dimensional reconstructions of spherical viruses by fourier synthesis from electron micrographs. Nature 226, 421-425.
Fuller, S., Butcher, S., Cheng, R., and Baker, T. S. (1996). Three-dimensional reconstruction of icosahedral particles—the uncommon line. J Struct Biol 116, 48-55.
Gaggar, A., Shayakhmetov, D. M., and Lieber, A. (2003). CD46 is a cellular receptor for group B adenoviruses. Nature Med 9, 1408-1412.
Heymann, J. (2001). Bsoft: image and molecular processing in electron microscopy. J Struct Biol 133, 156-169.
Hiltunen, M. O., Turunen, M. P., Turunen, A.-M., Rissanen, T. T., Laitinen, M., Kosma, V.-M., and Yla-Herttuala, S. (2000). Biodistribution of adenoviral vector to nontarget tissues after local in vivo gene transfer to arterial wall using intravascular and periadventitial gene delivery methods. FASEB J 14, 2230-2236.
Huard, J., Lochmuller, H., Ascadi, G., Jani, A., Massie, B., and Karpati, G. (1995). The route of administration is a major determinant of the transduction efficiency of rat tissues by adenoviral recombinants. Gene Ther 107, 107-115.
Kawabata, K., Sakurai, F., Koizumi, N., Hayakawa, T., and Mizuguchi, H. (2006) Adenovirus vector-mediated gene transfer into stem cells. Mol. Pharmaceut. 3, 95-103.
Koizumi, N., Kawabata, K., Sakurai, F., Watanabe, Y., Hayakawa, T. and Mizuguchi, H. Modified adenoviral vectors ablated for coxsackie-adenovirus, av integrin, and heparan sulphate binding reduce in vivo tissue transduction and toxicity. Hum Gene Ther 17, 264-279.
Kritz, A., Nicol, C., Dishart, K., Nelson, R., Holbeck, S., Von Seggern, D., Work, L., McVey, J., Nicklin, S., and Baker, A. (2007). Adenovirus 5 fibers mutated at the KKTK putative HSPG binding site show restricted retargeting capacity when engineered with targeting peptides in the HI loop. Mol Ther 15, 741-749.
Lecollinet, S., Gavard, F., Havenga, M J E., Spiller, O B., Lemckert, A., Goudsmit, J., Eloit, M., Richardson, J. (2006). Improved gene delivery to intestinal mucosa by adenoviral vectors bearing subgroup B and D Fibers. J Virol 80, 2747-2759.
Li, J., Lad, S., Yang, G., Luo, Y., Iacobelli-Martinez, M., Primus, F. J., Reisfeld, R. A., and L1, E. (2006). Adenovirus fiber shaft contains a trimerization element that supports peptide fusion for targeted gene delivery. J Virol 80, 12324-12331.
Madisch, I., Harste, G., Pommer, H., and Heim, A. (2005). Phylogenetic analysis of the main neutralization and hemagglutination determinants of all human adenovirus prototypes as a basis for molecular classification and taxonomy. J Virol 79, 15265-15276.
Martin, K., Brie, A., Sauliner, P., Perricaudet, M., Yep, P., and Vigne, E. 2003. Simultaneous CAR- and aᵥ integrin-binding ablation fails to reduce Ad5 liver tropism. Mol Ther 8, 485-494.
Monteiro, R., Rezaie, A., Ribeiro, J., and Francishetti, I. (2005). Ixolaris: a factor Xa heparin-binding exosite inhibitor. Biochem J 387, 871-877.
Murakami, M. T. R.-S., J; Weaver, S. E; Tulinsky, A; Geiger, J. H and Arni, R. K (2007). Intermolecular interactions and characterization of the novel factor Xa exosite involved in macromolecular recognition and inhibition: Crystal structure of human Gla-domainless factor Xa complexed with the anticoagulant protein NAPc2 from the hematophagous nematode Ancylostoma caninum J Mol Biol 366, 602-610.
Nicklin, S., Wu, E., Nemerow, G., and Baker, A. (2005). The influence of adenovirus fiber structure and function on vector development for gene therapy. Mol Ther 12, 384-393.
Nicol, C. G., Graham, D., Miller, W. H., White, S. J., Smith, T. A., Nicklin, S. A., Stevenson, S. C. and Baker, A. H. 2004. Effect of adenovirus serotype 5 fiber and penton modifications on iv vivo tropism in rats. Mol Ther 10, 344-354.
Oldstone, M., Lewicki, H., Thomas, D., Tishon, A., Dales, S., Patterson, J., Manchester, M., Homann, D., Naniche, D., Holz, A. (1999). Measles virus infection in a transgenic model: virus-induced immunosuppression and central nervous system disease. Cell 98, 629-640.
Parker, A. L., McVey, J. H., Doctor, J. H., Lopez-Franco, O., Waddington, S, N., Havenga, M. J. E., Nicklin, S. A., and Baker, A. H. (2007). Influence of coagulation factor zymogens on the infectivity of adenoviruses pseudotyped with fibers from subgroup D. J Virol 81, 3627-3631.
Parker, A. L., Waddington, S, N., Nicol, C. G., Shayakhmetov, D. M., Buckley, S. M., Denby, L., Kemball-Cook, G., Ni, S., Lieber, A., McVey, J. H., et al. (2006). Multiple vitamin K-dependent coagulation zymogens promote adenovirus-mediated gene delivery to hepatocytes. Blood 108, 2554-2561.
Pettersen, E., Goddard, T., Huang, C., Couch, G., Greenblatt, D., Meng, E., and Ferrin, T. (2004). UCSF Chimera—a visualization system for exploratory research and analysis. J Comput Chem 25, 1605-1612.
Pichia-Gollon, S. L. D., Mark; Zhou, Xiangyang; Xue, Feng; Rux, John J; Gao, Guang-Ping; Wilson, James M; Ertl, Hildegund C. J; Burnett, Roger M and Bergelson, Jeffrey M. (2007). Structure-based identification of a major neutralizing site in an adenovirus hexon. J Virol 81, 1680-1689.
Raper, S. E., Chirmule, N., Lee, F. S., Wivel, N. A., Bagg, A., Gao, G.-P., Wilson, J. M., and Batshaw, M. L. (2003). Fatal systemic inflammatory response syndrome in a ornithine transcarbamylase deficient patient following adenoviral gene transfer. Mol Genet Metab 80, 148-158.
Rezaie, A. R. (2000). Identification of basic residues in the heparin-binding exosite of factor Xa critical for heparin and factor Va binding. J Biol Chem 275, 3320-3327.
Roberts, D., Nanda, A., Havenga, M., Abbink, P., Lynch, D., Ewald, B., Liu, J., Thorner, A., Swanson, P., Gorgone, D., et al. (2006). Hexon-chimeric adenovirus serotype 5 vectors circumvent pre-existing anti-vector immunity. Nature 441, 239-243.
Roberts, M., White, J., Grutter, M., and Burnett, R. (1986). Three-dimensional structure of the adenovirus major coat protein hexon. Science 232, 1148-1151.
Rux, J. J., Pascolini, D., and Burnett R M (1999) Large-scale purification and crystallization of adenovirus hexon' in Methods in Molecular Medicine, Vol 21: Adenovirus Methods and Protocols (Ed WSM Wold) Humana Press Inc Totowa N.J. 259-275.
Rux, J., and Burnett, R. (2000). Type-specific epitope locations revealed by X-ray crystallographic study of adenovirus type 5 hexon. Mol Ther 1, 18-30.
Sakurai, F. K., K; Koizumi, N; Inoue, N; Okabe, M; Yamaguchi, T; Hayakawa, T; Mizuguchi, H (2006). Adenovirus serotype 35 vector-mediated transduction into human CD46-transgenic mice. Gene Ther 13, 1118-1126.
San Martin, C., and Burnett, R. (2003). Structural studies on adenoviruses. Curr Top Microbiol Immunol 272, 57-94.
Segerman, A., Atkinson, J. P., Marttila, M., Dennerquist, V., Wadell, G., and Arnberg, N. (2003). Adenovirus type 11 uses CD46 as a cellular receptor. J Virol 77, 9183-9191.
Shayakhmetov, D., Gaggar, A., Ni, S., L1, Z.-Y., and Lieber, A. (2005). Adenovirus binding to blood factors results in liver cell infection and hepatotoxicity. J Virol 79, 7478-7491.
Shayakhmetov, D., L1, Z., Ni, S., and Lieber, A. (2002). Targeting of adenovirus vectors to tumor cells does not enable efficient transduction of breast cancer metastases. Can Res 62, 1063-1068.
Shayakhmetov, D. M., Papayannopoulou, T., Stamatoyannopoulos, G., and Lieber, A. (2000). Efficient gene transfer into human CD34+ cells by a retargeted adenovirus vector. J Virol 74, 2567-2583.
Schenk-Braat, E. A. M, van Mierlo M. M. K. B., Wagemaker, G., Bangma, C. H., and Kaptein L. C. M. (2007) An inventory of shedding data from clinical gene therapy trials. J. Gene Med. 9, 910-921.
Smith, T. A. G., Idamakanti, N., Marshall-Neff, J., Rollence, M. L., Wright, P., Kaloss, M., King, L., Mech, C., Dinges, L., Iverson, W. O., et al. (2003). Receptor interactions involved in adenoviral-mediated gene delivery after systemic administration in non-human primates. Hum Gene Ther 14, 1595-1604.
Sumida, S. M., Truitt, D. M., Lemckert, A. A. C., Vogels, R., Custers, J. H. H. V., Addo, M. M., Lockman, S., Peter, T., Peyerl, F. W., Kishko, M. G., et al. (2005). Neutralizing antibodies to adenovirus serotype 5 vaccine vectors are directed primarily against the adenovirus hexon protein. J Immunol 174, 7179-7185.
Tomko, R. P., Xu, R., and Philipson, L. (1997). HCAR and MCAR: The human and mouse cellular receptors for subgroup C adenoviruses and group B coxsackieviruses. Proc Natl Acad Sci USA, USA 94, 3352-3356.
Tuve, S. W., Hongjie; W, Carol; L, Ying; Gaggar, A; Bernt, K; Shayakhmetov, D; L1, Z; Strauss, R; Stone, D. and Lieber, A. (2006). A new group B adenovirus receptor is expressed at high levels on human stem and tumor cells. J Virol 80, 12109-12120.
Van Rooijen, N. and A. Sanders. 1994. Liposome mediated depletion of macrophages: mechanism of action, preparation of liposomes and applications. J Immunol Methods 174, 83-93.
Venkateswarlu, D., Perera, L., Darden, T., and Pedersen, L. 2002. Structure and dynamics of zymogen human blood coagulation factor X. Biophys J 82, 1190-1206.
Vogels, R., Zuijeest, D., Rijnsoever, R. V., Hartkoorn, E., Damen, I., Bethune, M.-P. D., Kostense, S., Penders, G., Helmus, N., Koudstaal, W., et al. (2003). Replication-deficient human adenovirus type 35 vectors for gene transfer and vaccination: efficient human cell infection and bypass of preexisting adenovirus immunity. J Virol 77, 8263-8271.
Von Seggern, D. J., Chiu, C. Y., Fleck, S. K., Stewart, P. L., and Nemerow, G. R. A helper-independent adenovirus vector with E1, E3 and fiber deleted: Structure and infectivity of fiberless particles. J. Virol 73, 1601-1608.
Waddington, S., Parker, A., Havenga, M., Nicklin, S., Buckley, S., McVey, J., and Baker, A. (2007). Targeting of adenovirus serotype 5 (Ad5) and 5/47 pseudotyped vectors in vivo: A fundamental involvement of coagulation factors and redundancy of CAR binding by Ad5. J. Virol. 81: 9568-9571.
Wickham, T. J., Mathias, P., Cheresh, D. A., and Nemerow, G. R. (1993). Integrins aᵥb₃ and aᵥb₅ promote adenovirus internalization but not virus attachment. Cell 73, 309-319.
De Geest B, Snoeys J, Van Linthout S, Lievens J, Collen D: Elimination of innate immune responses and liver inflammation by PEGylation of adenoviral vectors and methylprednisolone. Hum Gene Ther 2005, 16:1439-1451.
O'Riordan C R, Lachapelle A, Delgado C, Parkes V, Wadsworth S C, Smith A E, Francis G E: PEGylation of adenovirus with retention of infectivity and protection from neutralizing antibody in vitro and in vivo. Hum Gene Ther 1999, 10:1349-1358.
Niu G, Xiong Z, Cheng Z, Cai W, Gambhir S S, Xing L, Chen X: In vivo bioluminescence tumor imaging of RGD peptide-modified adenoviral vector encoding firefly luciferase reporter gene. Mol Imaging Biol 2007, 9:126-134.
Lanciotti J, Song A, Doukas J, Sosnowski B, Pierce G, Gregory R, Wadsworth S, O'Riordan C: Targeting adenoviral vectors using heterofunctional polyethylene glycol FGF2 conjugates. Mol Ther 2003, 8:99-107.
Kim, J. S., Lee, S. J., Cho, Y. S., Choi, J. J., Kim, Y. H., Lee, J. H., (2002) Enhancement of the adenoviral sensitivity of human ovarian cancer cells by transient expression of coxsackievirus and adenovirus receptor (CAR). Gynecol Oncol 85(2), 260-265
Mizuno, H et al., (2001) Crystal structure of an anticoagulant protein in complex with the Gla domain of factor X. PNAS 98(13), 7230-7234
Bailey, W. L., D. W. LaFleur, et al. (1994). “Stimulation of rat vascular smooth muscle cell glycosaminoglycan production by angiotensin II.” Atherosclerosis 111: 55-64.
Rux, J. L., P. R. Kuser, et al. (2003). “Structural and phylogenetic analysis of adenovirus hexons by use of high-resolution x-ray crystallographic, molecular modeling, and sequence-based methods.” Virology 77: 9553-9566.
Waddington, S., J. et al. (2008). “Adenovirus serotype 5 hexon mediates liver gene transfer.” Cell 132, 397-409.
Wriggers, W., R. Milligan, et al. (1999). “Situs: A package for docking crystal structures into low-resolution maps from electron microscopy.” J Struct Biol 125: 185-95.

1-77. (canceled)

78. A method of modulating liver or spleen infectivity of an adenovirus comprising:
providing a parent adenoviral hexon protein;
modifying said parent hexon protein to produce a modified hexon protein having altered affinity for FX; and
producing an adenovirus comprising said modified hexon protein; whereby said adenovirus has altered ability to transduce hepatocytes or splenocytes compared to an adenovirus comprising said parent hexon protein, wherein the parent and modified hexon proteins differ:
(i) in one or more of HVR3 and HVR7;
(ii) by a point mutation in HVR5 at a residue corresponding to Thr268, Thr269 and/or Glu270 of Ad5 hexon protein [shown as Thr269, Thr270 and Glu271 in the sequence having accession nos. AAW65514.1; GI: 58177707];
(iii) by one or more amino acids in the scaffold sequence at residues corresponding to Ser267, Met314, Asn421, Thr426, Ser446, Asn449, Glu450, Arg452 and Val453 of Ad5 hexon protein [shown as Ser268, Met315, Asn422, Thr427, Ser447, Asn450, Glu451, Arg453 and Val454 in the sequence having accession nos. AAW65514.1; GI: 58177707]; and/or
(iv) in that the modified hexon protein is a chimeric hexon protein in which HVR5 is derived from a hexon protein of a different serotype to the parent hexon protein.

79. A modified adenoviral hexon protein, which differs from a parent hexon protein
(i) in one or more of HVR 3 and HVR7;
(ii) by a point mutation in HVR5 at a residue corresponding to Thr268, Thr269 and/or Glu270 of Ad5 hexon protein [shown as Thr269, Thr270 and Glu271 in the sequence having accession nos. AAW65514.1; GI: 58177707];
(iii) by one or more amino acids in the scaffold sequence at residues corresponding to Ser267, Met314, Asn421, Thr426, Ser446, Asn449, Glu450, Arg452 and Val453 of Ad5 hexon protein [shown as Ser268, Met315, Asn422, Thr427, Ser447, Asn450, Glu451, Arg453 and Val454 in the sequence having accession nos. AAW65514.1; GI: 58177707]; and/or
(iv) in that the modified hexon protein is a chimeric hexon protein in which HVR5 is derived from a hexon protein of a different serotype to the parent hexon protein; whereby an adenovirus comprising said modified hexon protein has altered ability to transduce hepatocytes or splenocytes compared to an otherwise identical adenovirus comprising said parent hexon protein.

80. The method of claim 78 wherein the parent and modified hexon proteins are identical apart from said differences.

81. The method according to claim 80 wherein the parent and modified adenoviruses are identical apart from said differences.

82. The method of claim 78 wherein the parent and modified hexon proteins differ by one or more mutations at residues corresponding to Ile420, Asn421, Thr422, Glu423, Thr424, Leu425, Asp447 or Lys448 of Ad5 hexon protein [shown as Ile421, Asn422, Thr423, Glu424, Thr425, Leu426, Asp448 and Lys449 in the sequence having accession nos. AAW65514.1; GI:58177707] located in HVR7, or Glu212, Thr213, Glu214, Ile215, Asn216, of Ad5 hexon protein [shown as Glu213, Thr214, Glu215, Ile216, Asn217, Ser268, Met315, Asn422, Thr427, Ser447 and Asn450 in the sequence having accession nos. AAW65514.1; GI=58177707] located in HVR3.

83. The method of claim 82 wherein the modified hexon protein contains Pro or Asp at a position at a position corresponding to Thr269, and/or Gly or Ser at a position corresponding to Glu270.

84. The method of claim 82 wherein the parent and modified hexon protein differ by mutations at residues corresponding to one, two three or all four of Ile420, Thr422. Glu423 and Leu425.

85. The method of claim 84 wherein the modified hexon protein contains Gly at a position corresponding to Ile420, Asn at a position corresponding to Thr422, Ser or Ala at a position corresponding to Glu423, and/or Tyr at a position corresponding to Leu425.

86. The method of claim 78 wherein the parent and modified hexon proteins differ by a mutation at a position corresponding to Glu450 of Ad5 [shown as Glu451 in the sequence provided below having accession nos. AAW65514.1; GI:58177707] and wherein the modified hexon protein carries a neutral or positively charged residue at that position, for example Gln, Ile, Leu, Val, Arg or Lys.

87. The method of claim 78 wherein the parent hexon protein is a wild type hexon protein and the modified hexon protein is a chimeric hexon protein having scaffold sequence from the parent hexon protein and at least one of HVRs 3, 5 and 7 from at least one different wild type hexon proteins.

88. The method of claim 87 wherein one or more of HVRs 3, 5 and 7 are derived from a different serotype than the scaffold.

89. The method of claim 88 wherein the parent hexon protein is of serotype Ad2 or Ad5 and one or more of HVRs 3, 5 and 7 of the modified hexon protein is derived from one or more of serotypes Ad17, Ad20, Ad25, Ad26, Ad28, Ad29, Ad44 or Ad48.

90. The method of claim 89 wherein the first hexon protein is of serotype Ad5 and one or both of HVR5 and HVR7 is derived from Ad26.

91. The method of claim 78 comprising providing a first nucleic acid sequence encoding said parent hexon protein, and modifying said first nucleic acid to generate a second nucleic acid sequence encoding said modified hexon protein.

92. A nucleic acid encoding a modified hexon protein according to claim 79.

93. An expression vector comprising a nucleic acid according to claim 92.

94. A host cell comprising a nucleic acid according to claim 92.

95. An adenovirus comprising a modified hexon protein according to claim 79.

96. A modified adenovirus produced by a method according claim 78.

97. An adenovirus according to claim 95 which is a gene delivery vector.

98-100. (canceled)

101. The hexon protein of claim 79 wherein the parent and modified hexon proteins are identical apart from said differences.

102. The hexon protein of claim 79 wherein the parent and modified hexon proteins differ by one or more mutations at residues corresponding to Ile420, Asn421, Thr422, Glu423, Thr424, Leu425, Asp447 or Lys448 of Ad5 hexon protein [shown as Ile421, Asn422, Thr423, Glu424, Thr425, Leu426, Asp448 and Lys449 in the sequence having accession nos. AAW65514.1; GI:58177707] located in HVR7, or Glu212, Thr213, Glu214, Ile215, Asn216, of Ad5 hexon protein [shown as Glu213, Thr214, Glu215, Ile216, Asn217, Ser268, Met315, Asn422, Thr427, Ser447 and Asn450 in the sequence having accession nos. AAW65514.1; GI=58177707] located in HVR3.

103. The hexon protein of claim 102 wherein the modified hexon protein contains Pro or Asp at a position at a position corresponding to Thr269, and/or Gly or Ser at a position corresponding to Glu270.

104. The hexon protein of claim 102 wherein the parent and modified hexon protein differ by mutations at residues corresponding to one, two three or all four of Ile420, Thr422. Glu423 and Leu425.

105. The hexon protein of claim 104 wherein the modified hexon protein contains Gly at a position corresponding to Ile420, Asn at a position corresponding to Thr422, Ser or Ala at a position corresponding to Glu423, and/or Tyr at a position corresponding to Leu425.

106. The hexon protein of claim 79 wherein the parent and modified hexon proteins differ by a mutation at a position corresponding to Glu450 of Ad5 [shown as Glu451 in the sequence provided below having accession nos. AAW65514.1; GI:58177707] and wherein the modified hexon protein carries a neutral or positively charged residue at that position, for example Gln, Ile, Leu, Val, Arg or Lys.

107. The hexon protein of claim 79 wherein the parent hexon protein is a wild type hexon protein and the modified hexon protein is a chimeric hexon protein having scaffold sequence from the parent hexon protein and at least one of HVRs 3, 5 and 7 from at least one different wild type hexon proteins.

108. The hexon protein of claim 107 wherein one or more of HVRs 3, 5 and 7 are derived from a different serotype than the scaffold.

109. The hexon protein of claim 108 wherein the parent hexon protein is of serotype Ad2 or Ad5 and one or more of HVRs 3, 5 and 7 of the modified hexon protein is derived from one or more of serotypes Ad17, Ad20, Ad25, Ad26, Ad28, Ad29, Ad44 or Ad48.

110. The hexon protein of claim 109 wherein the first hexon protein is of serotype Ad5 and one or both of HVR5 and HVR7 is derived from Ad26.


SEQUENCE LISTING (93 sequences)

SEQ ID NO: 1 - protein, 346 aa, Human adenovirus serotype 1
GAPNSCEWEQEEPTQEMAEELEDEEEAEEEEAEEEAEAPQADQKVKKTHVYAQAPLAGEKITANGLQIVSDTQTEGNPVFADPTYQPEPQVGESQWNEAEATASGGRVLKKTTPMKPCYGSYARPTNKNGGQGILVANNQGALESKVEMQFFAPSGTAMNERNAVQPSIVLYSEDVNMETPDTHISYKPSKTDENSKAMLGQQAMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGIGVTDTYQGIKSNGNGNPQNWTKNDDFAARNEIGVGNNFALE

SEQ ID NO: 2 - protein, 350 aa, Human adenovirus serotype 2
GAPNSCEWEQTEDSGRAVAEDEEEEDEDEEEEEEEQNARDQATKKTHVYAQAPLSGETITKSGLQIGSDNAETQAKPVYADPSYQPEPQIGESQWNEADANAAGGRVLKKTTPMKPCYGSYARPTNPFGGQSVLVPDEKGVPLPKVDLQFFSNTTSLNDRQGNATKPKVVLYSEDVNMETPDTHLSYKPGKGDENSKAMLGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGIGVTDTYQAIKANGNGSGDNGDTTWTKDETFATRNEIGVGNNFAME

SEQ ID NO: 3 - protein, 326 aa, Human adenovirus serotype 3
GAPNTSQWIVTTNRDNAVTTTTNTFGIASMKGDNITKEGLQIGKDITTTEGEEKPIYADKTYQPEPQVGEESWTDTDVTNEKFGGRALKPATNMKPCYGSFARPTNIKGGQAKNRKVKPTTEGGVETEEPDIDMEFFDGRDAVAGALAPEIVLYTENVNLETPDSHVVYKPGTSDNSHANLGQQAMPNRPNYIGFRDNFVGLMYYNSTGNMGVLAGQASQLNAVVDLQDRNTELSYQLLLDSLGDRTRYFSMWNQAVDSYDPDVRIIENHGIEDELPNYCFPLDGIGPGNRYQGIKVKTDDTNGWEKDANVATANEIAIGNNLAME

SEQ ID NO: 4 - protein, 318 aa, Human adenovirus serotype 4
GAPNTCQWKDSDSKMHTFGAAAMPGVTGKKIEADGLPIRIDSTSGTDTVIYADKTFQPEPQVGNDSWVDTNGAEEKYGGRALKDTTKMNPCYGSFAKPTNKEGGQANLKDSEPAATTPNYDIDLAFFDSKTIVANYDPDIVMYTENVDLQTPDTHIVYKPGTEDTSSESNLGQQAMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLNGVGLTDTYQGVKVKTDAGSEKWDKDDTTVSNANEIHVGNPFAME

SEQ ID NO: 5 - protein, 334 aa, Human adenovirus serotype 5
GAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAME

SEQ ID NO: 6 - protein, 345 aa, Human adenovirus serotype 6
GAPNSCEWEQNETAQVDAQELDEEENEANEAQAREQEQAKKTHVYAQAPLSGIKITKEGLQIGTADATVAGAGKEIFADKTFQPEPQVGESQWNEADATAAGGRVLKKTTPMKPCYGSYARPTNSNGGQGVMVEQNGKLESQVEMQFFSTSTNATNEVNNIQPTVVLYSEDVNMETPDTHLSYKPKMGDKNAKVMLGQQAMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGIGITDTFQAVKTTAANGDQGNTTWQKDSTFAERNEIGVGNNFAME

SEQ ID NO: 7 - protein, 316 aa, Human adenovirus serotype 7
GAPNTSQWIVTTGEDNATTYTFGIASTKGDNITKEGLEIGKDITADNKPIYADKTYQPEPQVGEESWTDIDGTNEKFGGRALKPATKMKPCYGSFARPTNIKGGQAKNRKVTPTEGDVEAEEPDIDMEFFDGREAADAFSPEIVLYTENVNLETPDSHVVYKPGTSDGNSHANLGQQAMPNRPNYIGFRDNFVGLMYYNSTGNMGVLAGQASQLNAVVDLQDRNTELSYQLLLDSLGDRSRYFSMWNQAVDSYDPDVRIIENHGVEDELPNYCFPLDGIGPGNKYQGIKPRDTAWEKDTKVSTANEIAIGNNLAME

SEQ ID NO: 8 - protein, 324 aa, Human adenovirus serotype 8
GAPNPSQWEQAKTGAGVDQNQKETRTYGVAATGGYNITKEGLQIGIDETKEDPNNKIYADKTFQPEPQIGENNWQDTNVFYGGRALKKETKMKPCYGSFARPTNKKGGQAKVLTTEDGQPTENFDIDLAFFDIPQAGGNDNLDPDMILYAENVNLETPDTHVVYKPGKDDASSAANLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGTGTNATYQGVEPDNAQGQNDKWKKDEKVAAQNQICKGNIYAME

SEQ ID NO: 9 - protein, 326 aa, Human adenovirus serotype 9
GAPNSSQWITKDTNAGNETTKTHTHGVAAMGGADITIKGLQIGVDRTENKNEPIYANEIYQPEPQVGEENLQDVENYYGGRALKKETKMKPCYGSFARPTNEKGGQAVKFLTDGDGQLTKNHDITMNFFDTPGDTNAEDTELEADIVMYTENVNMETPDTHVVYKPGPLEDSSEINLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGAGTNATYQGVKVKNGQDGDNNAEWEKDNAVADRNQICKGNIVAME

SEQ ID NO: 10 - protein, 331 aa, Human adenovirus serotype 10
SAPNPSQWTANEKQTGGQPKSVTQTFGSAPMGGSNITIEGLVIGTKEEEGNATEEIFADKTFQPEPQVGEENWQETEAFYGGRALKKDTKMKPCYGSFARPTNEKGGQAKLKLNAQGQPTKDYDIDLAFFDTPGGTPPTGSGQQEEYKADIIMYTENVNLETPDTHVVYKPGKEDESSETNLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLNGTGTNSTYQGVKVKTGQDGAEETEWDKDETVARQNQIAKGNVYAME

SEQ ID NO: 11 - protein, 321 aa, Human adenovirus serotype 11
GAPNPSQWTTKEKQTGVNAGDKEVTKTFGIAAMGGSNISENGLQIGTDTTADGTKPIYADKTFQPEPQVGEENWQDNDEYYGGRALKKDTKMKPCYGSFAKPTNKEGGQAKLKETPNGADPQYDVDMAFFDSTTINIPDVVLYTENVDLETPDTHVVYKPGKEDDSSEVNLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGIQTNSAYQGVKLKDNPTGGGANGDWVKDDTISAHNQIGKGNIFAME

SEQ ID NO: 12 - protein, 321 aa, Human adenovirus serotype 13; identical to SEQ ID NO: 11

SEQ ID NO: 13 - protein, 327 aa, Human adenovirus serotype 14
GAPNASQWLDKGVETTEERQNEDGENDEKATYTFGNAPVKADADITKDGLPIGLEVPAEGDPKPIYANKLYQPEPQVGQESWTDTDGTEEKYGGRVLKPDTKMKPCYGSFAKPTNVKGGQAKVKTEEGNNIEYDIDMNFFDLRSQKQGLKPKIVMYAENVDLESPDTHVVYKPEVSDASSNANLGQQSMPNRPNYIGFRDNFIGLMYYNSTGNMGVLAGQASQLNAVVDLQDRNTELSYQLLLDSLGDRTRYFSMWNQAVDSYDPDVRVIENHGVEDELPNYCFPLDGIGPRTDSYKEIQLNGDQAWKDVNPNGISELVKGNPFAME

SEQ ID NO: 14 - protein, 323 aa, Human adenovirus serotype 15
GAPNSSQWEQKKANAGDQKETHTYGVAPMGGENITISGLQIGTDTTNGKQDPIYANKLYQPEPQVGEENWQETEAFYGGRALKKETKMKPCYGSFARPTNEKGGQAKLRDPEKSQEDFDIVLAFFDTPGGTLTGGGTEYKADIVMCTENVNLETRDTHVVYKPGKDDDSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNAAYEGVKVKNGEDGDQESEWEKDTNVADRNQICKGNIYAME

SEQ ID NO: 15 - protein, 322 aa, Human adenovirus serotype 16
GAPNTCQWKDSDSKMHTFGVAAMPGVTGKKIEADGLPIGIDSTSGTDTVIYADKTFQPEPQVGNASWVDANGTEEKYGGRALKDTTKMKPCYGSFAKPTNKEGGQANLKDSETAATTPNYDIDLAFFDNKNIAANYDPDIVMYTENVDLQTPDTHIVYKPGTEDTSSESNLGQQAMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLNGVGFTDTYQGVKVKTDAVAGTSGTQWDKDDTTVSTANEIHGGNPFAME

SEQ ID NO: 16 - protein, 326 aa, Human adenovirus serotype 17
GAPNPSQWVAKENGQGTDKTHTYGSAAMGGSNITIEGLVIGTDEKAEDGKKDIFANKLYQPEPQVGEENWQESEAFYGGRALKKDTKMKPCYGSFARPTNEKGGQAKFKPVEEGQQPKDYDIDLAFFDTPGGTITGGTDEEYKADIVLYTENVNLETPDTHVVYKPGKEDDSSEVNLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLNGTGTNSTYLGVKVKPDQDGDVESEWDKDDTIARQNQIAKGNVFAME

SEQ ID NO: 17 - protein, 301 aa, Human adenovirus serotype 18
GAPNASQWLTTNDNKSHTFAQAPYIGSSITKDGIQVGTNTANPPQPVYADKTYQPEPQVGESQWNAPLPNNAKAAGRVLKNTTPMYPCYGSYARATNENGGQSKNNEGVEMQFFASAADNQNDPSVVLYSEDVNLEAPDTHIVFKPAVAADTVSSELLLGQQAAPNRPNYIGFRDNFIGLMYYNSTGNMGVLAGQASQLNAVVDLQDRNTELSYQLMLDALGDRSRYFSMWNSAVDSYDPDVRIIENHGVEDELPNYCFPLGAVGIVNSYKGVKYDANAWAKDNNKADNNNLATGNIFAME

SEQ ID NO: 18 - protein, 333 aa, Human adenovirus serotype 19
GAPNSSQWDAQEKNGQGGNDMVTKTHTFGVAAMGGTNITNQGLLIGTEETAHNPPKEIFADKLFQPEPQVGEENWQDTNAFYGGRALKKETKMKPCYGSYARPTNTSGGQAKLKTGDNIDPTKDFDIDLAFFDTPGGNPPAGGSGTEEYKADIVMYTENVNLETPDTHVVYKPGKEDESSEANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNAAYQGVKVQDGEDGDKETEWEKDTKVADRNQLCKGNIFAME

SEQ ID NO: 19 - protein, 325 aa, Human adenovirus serotype 20
GAPNSSQWLAKDTNAAGQPDKTHTYGVAAMGGEDITEKGLQIGIDETKEENNKIFANEIYQPEPHVGEENWQETFVFYGGRALKKDTKMKPCFGSFARPYYEKGGQAKFVLDQEGKPTKNHDITMAFFDTPGGQLNGKDELKADIVMYTENVNLETPDTHVVYKPGKEDDSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGAGTNAVYQGVKITDGNDGDVNDDWEKDTAVSERNQICKGNIYAME

SEQ ID NO: 20 - protein, 331 aa, Human adenovirus serotype 21
GAPNTSQWIAEGVKKEDGGSDEEEEKNLTTYTFGNAPVKAEGGDITKDKGLPIGSEITDGEAKPIYADKLYQPEPQVGDETWTDTDGTTEKYGGRALKPETKMKPCYGSFAKPTNVKGGQAKQKTTEQPQNQQVEYDIDMNFFDEASQKANFSPKIVMYAENVDLETPDTHVVYKPGTSEESSHANLGQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLDGVGVPISSYKIIEPNGQGADWKEPDINGTSEIGQGNLFAME

SEQ ID NO: 21 - protein, 317 aa, Human adenovirus serotype 22
GAPNSSQWAQKKTGEDNQTETRTFGVAAMGGILIDKNGLQIGTDETKPNNKEVYADKTFQPEPQIGEENWQDGDVFYGGRTIKKETKMKPCYGSFARPTNEKGGQAKFKTNAEGQPTEELDIDLNFFDINGGAGDNEFNPDMVMYAENMNLETPDTHVVYKPGTSDDSSEANLAQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGTGTNSTYQGVKETAAQNGWEKDPNVAAQNQICKGNIYAME

SEQ ID NO: 22 - protein, 313 aa, Human adenovirus serotype 23
GAPNPSQWEQKKTGVGPEAMEKHTFGMAAMAGEAITNKGLQIGVDTTDGKQDPIYANQLYQPEPQVGEDSWNDDVAPSYGGRALKKETKMKPCYGSFARPTNEKGGQAKLRDPEKSQEDFDIDMAFFDSNTINTPDVVLYTENVNLETPDSHVVYKAGTSDESSEINLGQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGVATNTVYQGVKLQTGQTDKWQKDDDVSTQNRIGKGSVFAME

SEQ ID NO: 23 - protein, 318 aa, Human adenovirus serotype 24
GAPNSSQWEQAKATNAGQKETHTFGVAAMGGEDITVKGLQIGTDETKEDGEDEIFADQTFQPEPQVGEQNWQETFVFYGGRALKKETKMKPCYGSYARPTNEKGGQAKFTLDEKGQPTKIPDITMDFFDSPQDDTSGVTNKPDIVMYAENVNLEAPDTHVVYKPGKDDSSSSANLTQQAMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLNGSGSNSTYKGVKAGTGNNWDDDENVARQNQIGTGNLFAME

SEQ ID NO: 24 - protein, 327 aa, Human adenovirus serotype 25
GAPNSSQWEEKKSGARNQTETHTFGVAPMGGTNITISGLQIGTEEEDGNPTKEIFADKTFQPEPQIGEENWQDTENFYGGRALKKDTKMKPCYGSFARPTNEKGGQAKLKLDAQGQSTKDYDIDLAFFDSPGGNTTAGGQEELKADIVMYTENAYLETPDTHVVYKPGTSDDSSAANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNSAYQGVKVKNGEDGDIESEWEKDTNVAARNQLCKGNIFAME

SEQ ID NO: 25 - protein, 334 aa, Human adenovirus serotype 26
GAPNPSQWETKEKQGTTGGVQQEKDVTKTFGVAATGGINITNQGLLLGTDETAENGKKDIYADKTFQPEPQVGEENWQENEAFYGGRALKKDTKMKPCYGSFARPTNEKGGQAKFKPVNEGEQPKDLDIDFAYFDVPGGSPPAGGSGEEYKADIILYTENVNLETPDTHVVYKPGTSDNSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLNGTGTNSTYQGVKITNGNDGAEESEWEKDDAISRQNQICKGNVYAME

SEQ ID NO: 26 - protein, 318 aa, Human adenovirus serotype 27
GAPNSSQWTAKENQKDVTKTFGVAPMGGINISKDGLQIGVEETVDKQEKEIYADKSFQPEPPVGKENWQESEAFYGGRALKKDTKMKPCYGSFARPTNEKGGQAKFKAPAVEGEQPKELDIDFAFFDTDSGDTEYKADIVMYAENVNLETPDTHVVYKPGKEDDSSEANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNSTYQGVKITDGNGALQNGWAKDDAISRQNQICKGNIYAME

SEQ ID NO: 27 - protein, 325 aa, Human adenovirus serotype 28
GAPNSSQWDAQEKSGQGSDMVTKTHTFGVAAMGGENITKNGLQIGTEITADNQKKEIFANKTYQPEPQVGEENWQENEVFYGGRALKKETKMKPCYGSFARPTNENGGQAKFKTPAEGQEPKELDIDLAFFDTDGGTADTEYKADIVMYAENVNLETPDTHVVYKPGKEDDSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGLGTNATYQGVKVSTGDGATQSGWAKDDTMARQNQICRGNIYAME

SEQ ID NO: 28 - protein, 323 aa, Human adenovirus serotype 29
GAPNSSQWEQKKANAGDQKETHTYGVAPMGGENITISGLQIGTDTTNGKQDPIYANKLYQPEPQVGEENWQETEAFYGGRALKKETKMKPCYGSFARPTNEKGGQAKLRDPEKSQEDFDIALAFFDTPGGTLTGGGTEYKADIVMCTENVNLETPDTHVVYKPGKDDDSSEINLGQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNAAYEGVKVKNGQDGDQESEWEKDTNVADRNQICKGNIYAME

SEQ ID NO: 29 - protein, 323 aa, Human adenovirus serotype 30
GAPNSSQWEQKKANAGDQKETHTYGVAPMGGENITISGLQIGTDTTNGKQDPIYANKLYQPEPQVGEENWQETEAFYGGRALKKETKMKPCYGSFARPTNEKGGQAKLRDPEKSQEDFDIDLAFFDTPGGTLTGGGTEYKADIVMCTENVNLETPDTHVVYKPGKDDDSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNAAYEGVKVKTGQDGDQETEWEKDTNVADRNQICKGNIYAME

SEQ ID NO: 30 - protein, 304 aa, Human adenovirus serotype 31
GAPNASQWLTTNNGNKTHTFAQAPYIGDSISKDGIQVGTNTANPQQAVYADKTYQPEPQVGESQWNASVSNNAKAAGRVLKSTTPMYPCYGSYARATNEKGGQSKNNDGVEMRFFASSANDNQNDPTVVMYSEDVNLEAPDTHIVYKPAVTADTISSELLLGQQAAPNRPNYIAFRDNFIGLMYYNSTGNMGVLAGQASQLNAVVDLQDRNTELSYQLMLDALGDRSRYFSMWNSAVDSYDPDVRIIENHGVEDELPNYCFPLGAVGVVNSYKGVKQNDGNGWVKDDSVADTNDIAKGNIFAME

SEQ ID NO: 31 - protein, 325 aa, Human adenovirus serotype 32
GAPNSSQWAAKDANTADQTVKTHTHGVAAMGGTDITAKGLQIGVDTTENNAGPIYANEIYQPEPQIGEENLQDVENYYGGRALKKETKMKPCYGSFARPTNEKGGQAKFITGEDGQPTKNHDITMNFFDTPGGTIGQADELEADIVMYAENVHLETPDTHVVYKPGTSDESSEANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGAGTNATYQGVKVKDGEDGNENADWVKDPNLASRNQICKGNIFAME

SEQ ID NO: 32 - protein, 325 aa, Human adenovirus serotype 33
GAPNSSQWEQKKATGGADAKETHTFGVAAMGGLNITDKGLQIGIDEDNVDGDDEIYADKTFQPEPQVGEENWKESFNFYGGRAIKKDTKMKPCYGSFARPTNVKGGQAKLKTGEDIDPNKDFDIDMAFFDLKQADTGGNNNQADVVMYTENIHLETPDTHVVYKPGKEDASSEINLTQQSMANRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGSSTAFQGVKVKTPAGTGQNTVWEVNNDAATHNQIARGNLYAME

SEQ ID NO: 33 - protein, 333 aa, Human adenovirus serotype 34
GAPNASQWLDKGVTSTGLVDDGNTDDGEEAKKATYTFGNAPVKAEAEITKDGLPVGLEVSTEGPKPIYADKLYQPEPQVGDETWTDLDGKTEEYGGRVLKPETKMKPCYGSFAKPTNIKGGQAKVKPKEDDGTNNIEYDIDMNFFDLRSQRSELKPKIVMYAENVDLECPDTHVVYKPGVSDASSETNLGQQSMPNRPNYIGFRDNFIGLMYYNSTGNMGVLAGQASQLNAVVDLQDRNTELSYQLLLDSLGDRTRYFSMWNQAVDSYDPDVRVIENHGVEDELPNYCFPLDGVGPRTDSYKEIKPNGDQSTWTNVDPTGSSELAKGNPFAME

SEQ ID NO: 34 - protein, 334 aa, Human adenovirus serotype 35
GAPNASQWIAKGVPTAAAAGNGEEEHETEEKTATYTFANAPVKAEAQITKEGLPIGLEISAENESKPIYADKLYQPEPQVGDETWTDLDGKTEEYGGRALKPTTNMKPCYGSYAKPTNLKGGQAKPKNSEPSSEKIEYDIDMEFFDNSSQRTNFSPKIVMYAENVGLETPDTHVVYKPGTEDTSSEANLGQQSMPNRPNYIGFRDNFIGLMYYNSTGNMGVLAGQASQLNAVVDLQDRNTELSYQLLLDSLGDRTRYFSMWNQAVDSYDPDVRVIENHGVEDELPNYCFPLDGIGVPTTSYKSIVPNGEDNNNWKEPEVNGTSEIGQGNLFAME

SEQ ID NO: 35 - protein, 326 aa, Human adenovirus serotype 36
GAPNSSQWTDKERQNGGQPPTTKDVTKTFGVAARGGLHITDKGLQIGEDENNEDGEEEIYADKTFQPEPQVGEENWQDTDVFYGGRALKKETKMKPCYGSFARPTNEKGGQAKFLNGENGQPSKDQDITLAFFDLKQNDTGTTQNQPDVVMYTENVYLETPDTHVVYKPGKEDTSSAANLTQQSMPNRPNYIGFRDNFVGLMYYNSTGNMGVLAGQASQLNAVVDLQDRNTELSYQLLLDSLGDRTRYFSMWNSTVDSYDPDVRIIENHGVEDELPNYCFPLDGSGSNTAYQGVKYENGAGNGSWKVDGEVASQNQIAKGNLYAME

SEQ ID NO: 36 - protein, 321 aa, Human adenovirus serotype 37
GAPNPSQWTTKEKQNGGTGAEKDVTKTFGLAAMGGSNISKDGLQIGTDKTANAEKPIYADKTFQPEPQVGEENWQDNDEYYGGRALKKDTKMKPCYGSFAKPTNKEGGQAKLKETPNGTDPQYDVDMAFFDSSTINIPDVVLYTENVDLETPDTHVVYKPGKEDDSSEANLTQQSMPNRPNYIGFRDNFVGLLYYNSTGNMGVLAGQASQLNAVVDLQDRNTELSYQLLLDSLGDRTRYFSMWNSAVDSYDPDVRIIENHGVEDELPNYCFPLDGVQTNSAYQGVKLKPDQTGGGVNGDWVKDDDISAHNQIGKGNIFAME

SEQ ID NO: 37 - protein, 322 aa, Human adenovirus serotype 38
GAPNSSQWDAKEKNGGAETTKTYTYGVAAMGGLDITDKGLQIGTDETKEDNNEIFADKTFQPEPQVGEETWQENEVFYGGRALKKETKMKPCYGSFARPTNEKGGQAKFLNDDQGLPSKEQDITMAFFDSPQADTSGVDNKPDMVMYTENVYLETPDTHVVFKPGKEDDSSEVNLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGSSSTYQGVKYENGAGGNGTWKVDDTVTRQNQIAKGNLYAME

SEQ ID NO: 38 - protein, 321 aa, Human adenovirus serotype 39
GAPNPSQWIAKEKQNGPPQTEKNVTKTFGVAAMGGLDITNEGLQIGVEEINDVEEEVFADKTFQPEPQVGEENWHETFNFYGGRTLKKETKMKPCYGSFARPTNIKGGQAKLKTGENVDPTKDFDIDMAFFDLKQTDTGTTQNQPDIVMYTENVNLETPDTHVVYKPGKEDASSEINLTQQSMANRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGMGSNAAYQGVKPKTGNGWDPDTDVAAQNQIAKGNIYAME

SEQ ID NO: 39 - protein, 305 aa, Human adenovirus serotype 40
GAPNPSQWTNQNKTNSFGQAPYIGQKITNQGVQVGLDSNNRDVFADKTYQPEPQVGQTQWNINPMQNAAGRILKQTTPMQPCYGSYARPTNEKGGQAKLVKNDDNQTTTTNVGLNFFTTATETANFSPKVVLYSEDVNLEAPDTHLVFKPDVNGTSAELLLGQQAAPNRPNYIGFRDNFIGLMYYNSTGNMGVLAGQASQLNAVVDLQDRNTELSYQLMLDALGDRSRYFSMWNQAVDSYDPDVRIIENHGVEDELPNYCFPLNGQGISNSYQGVKTDNGTNWSQNNTDVSSNNEISIGNVFAME

SEQ ID NO: 40 - protein, 330 aa, Human adenovirus serotype 42
GAPNSSQWADKERVNGGGNTKDVTKTFGVAAMGGEDITEKGLKIGTDPTANEPIFADKNFQPEPQVGEENWQETFVFYGGRALKKETKMKPCYGSFARPTNEKGGQAKFIIGDNGQPTENHDITMAFFDTPGGTITGGTGGPQDELKADIVMYTENINLETPDTHVVYKPGKEDDSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNSAFQGVKIKQNQDGDVNDDWEKDDKVSTQNQICKGNIYAME

SEQ ID NO: 41 - protein, 323 aa, Human adenovirus serotype 43
GAPNSSQWETKEKQNGGSGAQIEKNVTKTFGVAAMGGLDITDEGLQIGVEEINNVEEEVFADKIFQPEPQVGEENWQETFNFYGGRALKKDTKMKPCYGSFARPTNEKGGQAKLKTGENVDPTKDFDIDMAFFDLKQTDTGTTQNQPDIVMYTENVNLETPDTHVVYKPGKEDASSEINLTQQSMANRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGMGSNAAYQGVKPKTGNGWDPNTDVAAQNQIAKGNIYAME

SEQ ID NO: 42 - protein, 320 aa, Human adenovirus serotype 44
GAPNPSQWEQKKNGGGADQMETRTYGVAAMGGIDIDKNGLQIGVEQTADNGQKEIYADKLFQPEPQIGEENWHETFVYYGGRALKKDTKMKPCYGSFARPTNEKGGQAKPKTGEGVDPTKDFDIDLAFFDIPQNGVQDNHDPDMIMYAENVNLETPDTHIVYKPGKEDESSEANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGAGTNAVYQGVKEQLNQNDKWEVDNAISNQNRICKGNVYAME

SEQ ID NO: 43 - protein, 331 aa, Human adenovirus serotype 45
SAPNPSQWDAKEKEGVAQTEKNVLKTFGVAATGGFNITDQGLLLGTEETAENVKKDIYAEKTFQPEPQVGEENWQESEAFYGGRAIKKDTKMKPCYGSFARPTNEKGGQAKFKTLDGQVTKDPDIDFAYFDVPGGKAPTGSSLPEEYKADIILYTENVNLETPDTHIVYKPGKEDDNSEINLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLNGTGTNSTYQGVKITGNNDGDLETEWERDEAISRQNQICKGNVYAME

SEQ ID NO: 44 - protein, 319 aa, Human adenovirus serotype 46
GAPNSSQWEQKKANGGPNEMETHTFGVAAMGGENITKDGLQIGTETTAENQNKEIFADKTFQPEPQVGEENWQETFNFYGGRALKKETKMKPCYGSFARPMNEKGGQAKFLTKENGELTEDQDIDLNFFDINNPDTGGVANQPDIIMYAENVNLETPDTHVVYKSGKEDDSSEANLLQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLNGMGSNAAYQGVKPKDNGGWDPNTNAARQNRIAMGNVYAME

SEQ ID NO: 45 - protein, 328 aa, Human adenovirus serotype 47
GAPNPSQWDAQENNAGDAKTHTYGVASMPGIDITDKGLQIGIDANKDEDEGNEIFADKTFQPEPQVGEENWQESENFYGGRALKKETKMKPCYGSFARPTNEKGGQAKFKTPDKEGEQPKEYDIDMNFFDIPNTGTGGNGTNVNNKPDIVLYAENMNLETPDTHVVYKPGKEDDSSEVNLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGTGTASAYQGVKVKNGTVAGKVEWDPDTKVASKNRICKGSIFAME

SEQ ID NO: 46 - protein, 329 aa, Human adenovirus serotype 48
GAPNPSQWEEKKNGGGSDANQMQTHTFGVAAMGGIEITAKGLQIGIDATKEEDNGKEIYADKTFQPEPQIGEENWQDSDNYYGGRAIKKETKMKPCYGSFARPTNEKGGQAKFKTPEKEGEEPKELDIDLNFFDIPSTGTGGNGTNVNFKPDMIMYAENVNLETPDTHIVYKPGKEDASSESNLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGAGTNAVYQGVKVKTTNNTEWEKDTAVSEHNQICKGNVYAME

SEQ ID NO: 47 - protein, 325 aa, Human adenovirus serotype 49
GAPNSSQWDAKENNGQGEAKTHTYGVAAMGGYNITKDGLQIGIDENKEEDEEGREIFAVKSYQPEPQVGEENWQNTENFYGGRALKKETKMKPCYGSFARPTNDKGGQAVFKTGENGKPTEELDIDLAFFDLRQNDTGGNNNQPDMIMYAENVNLETPDTHVVYKPGTSDDSSEINLCQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGSSTAYQGVEPDTTVAGTNDKWKVNAKVAQHNQIAKGNLFAME

SEQ ID NO: 48 - protein, 322 aa, Human adenovirus serotype 50
GAPNTSQWLNKGDEEDGEDDQQATYTFGNAPVKAEAEITKEGLPIGLEVPSEGGPKPIYADKLYQPEPQVGEESWTDTDGTDEKYGGRALKPETKMKPCYGSFAKPTNVKGGQAKVKKEEEGKVEYDIDMNFFDLRSQMTGLKPKIVMYAENVDLETPDTHVVYKPGASDASSHANLGQQSMPNRPNYIGFRDNFIGLMYYNSTGNMGVLAGQASQLNAVVDLQDRNTELSYQLLLDSLGDRTRYFSMWNQAVDSYDPDVRVIENHGVEDELPNYCFPLDGVGPRIDSYKGIETNGDETTTWKDLEPKGISEIAKGNPFAME

SEQ ID NO: 49 - protein, 328 aa, Human adenovirus serotype 51
GAPNSSQWEQKKTTGGGNDMETHTFGVAAMGGENITKDGLQIGTDTTADADKPIYADKTFQPEPQVGEENWQETFNFYGGRALKKDTKMKPCYGSFARPTNEKGGQAKLKNGPDGKPTKEFDIDLAFFDTPGGKLPGNDQKEEYKADIVMYTENAYLETPDTHVVYKPGKDDASSEANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNAAYQGVKVTEGEDGAEESEWELENKLAARNQICKGNIFAME

SEQ ID NO: 50 - protein, 337 aa, Artificial Sequence
  Synthetic sequence Ad5HVR48 mutant
GAPNPCEWDEAATALEIEKKNGGGSDANQMQTHVFGQAPYSGINITKEGLQIGIDATKEEDNGKEIYADKTFQPEPQIGESQWQDSDNYYGGRVLKKTTPMKPCYGSYAKPTNENGGQAKFKTPEKEGEEPKESQVEMQFFDIPSTGTGGNGTNVNFKPKVVLYSEDVDIETPDTHISYMPGKEDASSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLDGAGTNAVYQGVKVKTTNNTEWEKDTAVSEHNQIRVGNNFAME

SEQ ID NO: 51 - protein, 448 aa, Homo sapiens [sequence omitted]

SEQ ID NO: 52 - protein, 441 aa, Mus musculus
ANSFFEEFKKGNLERECMEEICSYEEVREIFEDDEKTKEYWTKYKDGDQCESSPCQNQGACRDGIGGYTCTCSEGFEGKNCELFVRKLCRLDNGDCDQFCREEQNSVVCSCASGYFLGNDGKSCISTAPFPCGKITTGRRKRSVALNTSDSELDLEDALLDEDFLSPTENPIELLNLNETQPERSSDDLVRIVGGRECKDGECPWQALLVNEDNEGFCGGTILNEFYILTAAHCLHQARRFKVRVGDRNTEKEEGNEMVHEVDVVIKHNKFQRDTYDYDIAVLRLKTPITFRMNVAPACLPQKDWAESTLMTQKTGIVSGFGRTHEKGRQSNILKMLEVPYVDRNTCKLSTSFSITQNMFCAGYEAKLEDACQGDSGGPHVTRFKNTYYVTGIVSWGEGCARKGKYGIYTKVTTFLKWIDRSMKARVGPTAETPRTAGPPN

SEQ ID NO: 53 - protein, 448 aa, Macaca mulatta
SNSFLEEMKKGNLERECMEETCSYEEAREVLEDSDKTNEFWNKYKDGDQCETSPCQNEGKCRDGLGEYTCTCLEGFEGKNCELFTRKLCSLDNGECDQFCHEEQNSVVCSCARGYTLADNGKACIPTGPYPCGKQTLERRKRSAAQATNSSGEAPDNIIWKPDDAADLDATENPFDLLDFNQTQPERGDNNLIRIVGGRECENGECPWQALLINEENEGFCGGTILSEFYILTAAHCLYQAKRFKVRVGDRDMEQEEGGEAVHEVEVIIKHNRFTKETYDFDIAVLRLKSPITFRMNVAPACLPERDWAESTLMTQKTGIVSGFGRTHEKGRQSTRLKMLEVPYVDRNSCKLSSSFIITQNMFCAGYHAKQEDACQGDSGGPHVTRFKDTYFVTGIVSWGEGCARKGKYGIYTKVTAFLKWIDRSMKTRGLPKAESRAPEASTSSPLK

SEQ ID NO: 54 - protein, 448 aa, Papio anubis
SNSFLEELKKGNLERECMEETCSYEEAREVFEDIDKTNEFWNKYKDGDQCETSPCQNEGKCRDGLGEYTCTCLEGFEGKNCELFTRKLCSLDNGDCDQFCHEEQNSVVCSCARGYTLADNGKACIPTGPYPCGKQTLERRKRSAAQATNSSGEASDSIIWKPDDAADLDATENPFDLLDFNQTQPERGDNNLIRIVGGRECENGECPWQALLINEENEGFCGGTILSEFYILTAAHCLYQAKRFKVRVGDRDMEQEEGGEAVHEVEVIIKHNRFTKETYDFDIAVLRLKSPITFRMNVAPACLPERDWAESTLMTQKTGIVSGFGRTHEKGRQSTRLKMLEVPYVDRNSCKLSSSFIITQNMFCAGYHAKQEDACQGDSGGPHVTRFKDTYFVTGIVSWGEGCARKGKYGIYTKVTAFLKWIDRSMKTRGLPKAESRAPEVSTSSPLK

SEQ ID NO: 55 - protein, 448 aa, Callithrix jacchus
ANSILEELKKGNLERECMEETCSYEEAREVFEDSDQTNEFWNKYKDGDQCESSPCQNQGKCRDGLGQYTCTCLEGFEGKNCELFTRKLCSLDNGDCDQFCHEEQNSVVCSCASGYILADDGKACIPTGPYPCGKLTLERRKRSVAQATNSSGETPDTVTLPPNNTSDLDPTENPFDLLGFNQTQPEGEDNDLVRIVGGRECKDGECPWQALLINEENEGFCGGTILSEFYVLTAAHCLHQAKRFKVRVGDRDTEKEEGGETVHEVEVIIKHNRFSIETYDFDITVLRLKTPITFRMNVAPACLPARDWAESTLMTQKTGIVSGFGRTHEKGRQSTTLKILEVPYVDRNTCKLSSSFTITQNMFCAGYEARQEDACQGDSGGPHVTRFKDTYFVTGIISWGEGCARKGKYGIYTKVSGFLKWIDRSMKTRGVAKAESQVLGVNTSSPLK

SEQ ID NO: 56 - protein, 446 aa, Canis familiaris
ANSFLEEMKKGNLERECMEETCSFEEAREVFEDTAKTMEFWNKYKDGDQCESSPCQNQGQCKDGLLEYSCICLEGYEGKNCELSTRKLCSVDNGDCDQFCREEQSSVVCSCASGYILGDNGKSCISTEPFPCGKTTVGRRKRATETAPSSEAPPDAEEEAGMLEQYDPGDLSPTQSTMFLLPFNQTNSDPDEDASGLVRIVGGQDCRDGECPWQALLINEENEGFCGGTILSEYYILTAAHCLQQAKKFTVRVGERDTDKEEGNEVAHEVEMIIKHNKFVRETYDFDIAVIKLKTPITFRMNVAPACLPQKDWAESTLMTQKTGIVSGFGKTHEKGRPSTTLKMMEVPYVDRNTCKLSSSFSITQNMFCAGYDSKPEDACQGDSGGPHVTRFKDTYFVTGIVSWGEGCARKGKYGIYTKVTNFLKWIDRSMKARGAAPALAGGPHT

SEQ ID NO: 57 - protein, 442 aa, Rattus norvegicus
ANSFFEEIKKGNLERECVEEICSFEEAREVFEDNEKTTEFWNKYEDGDQCESSPCQNQGECRDGLGSYTCTCTEGFEGKNCELFVRKLCSLDNGDCDQFCREEQNSVVCSCAKGYFLGNDGKSCLSTAPFPCGKTNKGRAKRSVALNTSNSEPDPEDLMPDADILYPTESPSELLNLNKTEPEANSDDVIRIVGGQECKRGECPWQALLFSDEETDGFCGGTILNEFYILTAAHCLHQAKRFKVRVGDLNTEQEDGGEMVHEVDMIIKHNKFQRDTYDFDIAMLRLKTPITFRENVAPACLPQKDWAEATLMTQKTGIVSGFGRTHEKGRQSKVLKMMEVPYVDRNTCRLSTSFSITQNMFCAGYDAKQEDACQGDSGGPHVTRFKDTYFVTGIVSWGEGCARKGKYGIYTKVTAFLKWIDRSMKARVGPTSETPRLTHPPY

SEQ ID NO: 58 - protein, 439 aa, Sus scrofa
ANSFWEEMKKGNLKRECLEETCSYEEAREVFEDQQKTDEFWNKYKDGDQCESSPCQNQGKCKDGLGEYNCTCAEGFEGKNCELLTRKLCSLDNGDCDQFCREEQSSVVCSCASGYILGDNGKSCISTEPFPCGKRTLGRSRRSANHGSEAAAEAGRSEQEPGDLGPTEGPLHLLSLNDTEPGPEEDEDESSLVRIVGGRDCREGECPWQALLVNEENEGFCGGTTLSEFHVLTAAHCLHQAKRFKVRVGDHNLEKEEGDEAAHEVEVTVKHSRFVRETYDFDIAVLKLKTPITFRTNVAPACLPEKNWAEATLLTQKTGIVSGFGRTHERGRPSSTLKMLEVPYVDRNTCKLSSSFLITPNMFCAGYDEQPEDACQGDSGGPHVTRFRDTYFVTGIVSWGEGCARRGKYGVYTKVTNFLKWIERSMRARAEAGAEPHPQ

SEQ ID NO: 59 - protein, 450 aa, Oryctolagus cuniculus
ANSFLEELKKGNLERECMEENCSYEEALEVFEDREKTNEFWNKYVDGDQCESNPCQNQGTCKDGLGMYTCSCVEGYEGQDCEPVTRKLCSLDNGGCDQFCKEEENSVLCSCASGYTLGDNGKSCISTELFPCGKVTLGRWRRSPATNSSEGPPEAPGPEQQDDGNLTATENPFNLLDSPEPPPEDDSSSLVRIVGGQDCRDGECPWQALLVNEENEGFCGGTILSEYHVLTAAHCLHQAKRFKVRVGDRDTEHEEGNEETHEVEVVVKHNRFVKETYDFDIAVLRLKTPITFRRNVAPACLPQKDWAESTLMAQKTGIVSGFGRTHEMGRLSTTLKMLEVPYVDRNSCKRSSSFTITQNMFCAGYDARPEDACQGDSGGPHVTRFRDTYFVTGIVSWGEGCARKGKFGVYTKVSNFLKWIEKSMRARAVPVAEAAGTPGPTQPTIKGSPS

SEQ ID NO: 60 - protein, 452 aa, Bos taurus
ANSFLEEVKQGNLERECLEEACSLEEAREVFEDAEQTDEFWSKYKDGDQCEGHPCLNQGHCKDGIGDYTCTCAEGFEGKNCEFSTREICSLDNGGCDQFCREERSEVRCSCAHGYVLGDDSKSCVSTERFPCGKFTQGRSRRWAIHTSEDALDASELEHYDPADLSPTESSLDLLGLNRTEPSAGEDGSQVVRIVGGRDCAEGECPWQALLVNEENEGFCGGTILNEFYVLTAAHCLHQAKRFTVRVGDRNTEQEEGNEMAHEVEMTVKHSRFVKETYDFDIAVLRLKTPIRFRRNVAPACLPEKDWAEATLMTQKTGIVSGFGRTHEKGRLSSTLKMLEVPYVDRSTCKLSSSFTITPNMFCAGYDTQPEDACQGDSGGPHVTRFKDTYFVTGIVSWGEGCARKGKFGVYTKVSNFLKWIDKIMKARAGAAGSRGHSEAPATWTVPPPLPL

SEQ ID NO: 61 - protein, 401 aa, Human adenovirus serotype 5
VDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRN

SEQ ID NO: 62 - protein, 398 aa, Human adenovirus serotype 35
VDREDNTYSYKVRYTLAVGDNRVLDMASTFFDIRGVLDRGPSFKPYSGTAYNSLAPKGAPNASQWIAKGVPTAAAAGNGEEEHETEEKTATYTFANAPVKAEAQITKEGLPIGLEISAENESKPIYADKLYQPEPQVGDETWTDLDGKTEEYGGRALKPDTKMKPCYGSFAKPTNVKGGQAKQKTTEQPNQKVEYDIDMEFFDAASQRTNFSPKIVMYAENVGLETPDTHVVYKPGTSSEANLGQQSMPNRPNYIGFRDNFIGLMYYNSTGNMGVLAGQASQLNAVVDLQDRNTELSYQLLLDSLGDRTRYFSMWNQAVDSYDPDVRVIENHGVEDELPNYCFPLNGIGVPTTSYKSIVPNGEDNNNWKEPEVNGTSEIGQGNLSAMEINLQANLWRS

SEQ ID NO: 63 - protein, 389 aa, Human adenovirus type 50
VDREDNTYAYKVRYTLAVGDNRVLDMASTFFDIRGVLDRGPSFKPYSGTAYNSLAPKGAPNTSQWLNKGDEEDGEDDQQATYTFGNAPVKAEAEITKEGLPIGLEVPSEGGPKPIYADKLYQPEPQVGEESWTDTDGTDEKYGGRALKPETKMKPCYGSFAKPTNVKGGQAKVKKEEEGKVEYDIDMNFFDLRSQMTGLKPKIVMYAENVDLETPDTHVVYKPGASDASSHANLGQQSMPNRPNYIGFRDNFIGLMYYNSTGNMGVLAGQASQLNAVVDLQDRNTELSYQLLLDSLGDRTRYFSMWNQAVDSYDPDVRVIENHGVEDELPNYCFPLDGVGPRIDSYKGIETNGDETTTWKDLEPKGISEIAKGNPFAMEINLQANLWRS

SEQ ID NO: 64 - protein, 392 aa, Human adenovirus serotype 49
VDREDTTYSYKARFTLAVGDNRVLDMASTYFDIRGVLDRGPSFKPYSGTAYNSLAPKGAPNSSQWDAKENNGQGEAKTHTYGVAAMGGYNITKDGLQIGIDENKEEDEEGREIFAVKSYQPEPQVGEENWQNTENFYGGRALKKETKMKPCYGSFARPTNDKGGQAVFKTGENGKPTEELDIDLAFFDLRQNDTGGNNNQPDMIMYAENVNLETPDTHVVYKPGTSDDSSEINLCQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGSSTAYQGVEPDTTVAGTNDKWKVNAKVAQHNQIAKGNLFAMEINLQANLWKS

SEQ ID NO: 65 - protein, 401 aa, Human adenovirus serotype 26
VDREATTYLYKARFTLAVGDNRVLDMASTYFDIRGVLDRGPSFKPYSGTAYNSLAPKGAPNASQWIAKGVPTAAAAGNGEEEHETEEKTATYTFANAPVKAEAQITKEGLPIGLEISAENESKPIYADKLYQPEPQVGDETWTDLDGKTEEYGGRALKPTTNMKPCYGSYAKPTNLKGGQAKPKNSEPSSEKIEYDIDMEFFDNSSQRTNFSPKIVMYAENVGLETPDTHVVYKPGTEDTSSEANLGQQSMPNRPNYIGFRDNFIGLMYYNSTGNMGVLAGQASQLNAVVDLQDRNTELSYQLLLDSLGDRTRYFSMWNQAVDSYDPDVRVIENHGVEDELPNYCFPLDGIGVPTTSYKSIVPNGEDNNNWKEPEVNGTSEIGQGNLFAMEINLQANLWKS

SEQ ID NO: 66 - protein, 45 aa, Human adenovirus serotype 5
EAATALEINLEEEDDDNEDEVDEQAEQQKTHVFGQAPYSGINITK

SEQ ID NO: 67 - protein, 7 aa, Human adenovirus serotype 5
GVEGQTP

SEQ ID NO: 68 - protein, 9 aa, Human adenovirus serotype 5
QWYETEINH

SEQ ID NO: 69 - protein, 13 aa, Human adenovirus serotype 5
GILVKQQNGKLES

SEQ ID NO: 70 - protein, 16 aa, Human adenovirus serotype 5
FSTTEATAGNGDNLTP

SEQ ID NO: 71 - protein, 12 aa, Human adenovirus serotype 5
PTIKEGNSRELM

SEQ ID NO: 72 - protein, 25 aa, Human adenovirus serotype 5
INTETLTKVKPKTGQENGWEKDATE

SEQ ID NO: 73 - protein, 33 aa, Human adenovirus serotype 26
TKEKQGTTGGVQQEKDVTKTFGVAATGGINITN

SEQ ID NO: 74 - protein, 12 aa, Human adenovirus serotype 26
GTDETAENGKKD

SEQ ID NO: 75 - protein, 7 aa, Human adenovirus serotype 26
NWQENEA

SEQ ID NO: 76 - protein, 14 aa, Human adenovirus serotype 26
AKFKPVNEGEQPKD

SEQ ID NO: 77 - protein, 26 aa, Human adenovirus serotype 26
LDIDFAYFDVPGGSPPAGGSGEEYKA

SEQ ID NO: 78 - protein, 12 aa, Human adenovirus serotype 26
PGTSDNSSEINL

SEQ ID NO: 79 - protein, 28 aa, Human adenovirus serotype 26
GTNSTYQGVKITNGNDGAEESEWEKDDA

SEQ ID NO: 80 - protein, 30 aa, Human adenovirus serotype 48
EKKNGGGSDANQMQTHTFGVAAMGGIEITA

SEQ ID NO: 81 - protein, 13 aa, Human adenovirus serotype 48
GIDATKEEDNGKE

SEQ ID NO: 82 - protein, 7 aa, Human adenovirus serotype 48
NWQDSDN

SEQ ID NO: 83 - protein, 15 aa, Human adenovirus serotype 48
AKFKTPEKEGEEPKE

SEQ ID NO: 84 - protein, 19 aa, Human adenovirus serotype 48
FDIPSTGTGGNGTNVNFKP

SEQ ID NO: 85 - protein, 12 aa, Human adenovirus serotype 48
PGKEDASSESNL

SEQ ID NO: 86 - protein, 24 aa, Human adenovirus serotype 48
GTNAVYQGVKVKTTNNTEWEKDTA

SEQ ID NO: 87 - protein, 952 aa, Human adenovirus serotype 5
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 88 - protein, 129 aa, Deinagkistrodon acutus
DCSSGWSSYEGHCYKVFKQSKTWADAESFCTKQVNGGHLVSIESSGEADFVGQLIAQKIKSAKIHVWIGLRAQNKEKQCSIEWSDGSSISYENWIEEESKKCLGVHIETGFHKWENFYCEQQDPFVCEA

SEQ ID NO: 89 - protein, 123 aa, Deinagkistrodon acutus
DCPSDWSSYEGHCYKPFNEPKNWADAENFCTQQHTGSHLVSFQSTEEADFVVKLAFQTFDYGIFWMGLSKIWNQCNWQWSNAAMLKYTDWAEESYCVYFKSTNNKWRSITCRMIANFVCEFQA

SEQ ID NO: 90 - DNA, 19 bp, Artificial sequence
  Synthetic sequence Primer
acagtggaaaggtcgacgc

SEQ ID NO: 91 - DNA, 20 bp, Artificial sequence
  Synthetic sequence Primer
acttgactttctagctttcc

SEQ ID NO: 92 - DNA, 16 bp, Artificial sequence
  Synthetic sequence Oligonucleotide
cgcggtgcggctggtg

SEQ ID NO: 93 - DNA, 18 bp, Artificial sequence
  Synthetic sequence Oligonucleotide
tggcgcatcccattctcc